(19) United States
(12) Patent Application Publication
(10) Pub. No.: US 2025/0002967 A1
(43) Pub. Date: Jan. 2, 2025

(54) METHODS FOR DETECTING MICROORGANISMS BY ADDING A FLUORESCENT DYE DIRECTLY TO THE SOLID GROWTH MEDIUM

(71) Applicant: DIAMIDEX, MARSEILLE CEDEX 09 (FR)

(72) Inventors: ALEXANDRE MULLER, MARSEILLE (FR); AUDREY DUMONT, AUBAGNE (FR); SAM DUKAN, MARSEILLE (FR)

(21) Appl. No.: 18/569,620

(22) PCT Filed: Jun. 14, 2022
(86) PCT No.: PCT/EP2022/066238

§ 371 Date: Dec. 13, 2023

(30) Foreign Application Priority Data
Jun. 14, 2021 (EP) 21305813.4
Oct. 15, 2021 (EP) 21306443.9

(51) Int. Cl. C12Q 1/04; C12Q 1/06

(52) U.S. Cl. CPC C12Q 1/045; C12Q 1/06

(57) ABSTRACT

The present invention relates to a method for detecting and/or enumerating a microorganism or a group of microorganisms of interest comprising a) contacting, in a container, a sample suspected of containing said microorganism or group of microorganisms with a solid growth medium containing nutrients to support growth of said microorganism or group of microorganisms and with at least one fluorescent dye; b) incubating said container under conditions and for a time sufficient to form microcolonies of said microorganism or group of microorganisms; and c) detecting and/or enumerating microcolonies emitting a fluorescent signal of said at least one fluorescent dye, thereby detecting, identifying and/or enumerating said microorganism or group of microorganisms contained in the sample.

### CROSS-REFERENCE TO RELATED APPLICATION

This application is the U.S. national stage application of International Patent Application No. PCT/EP2022/066238, filed Jun. 14, 2022.

### FIELD OF THE INVENTION

The present invention relates to the field of microbiology and more specifically to a method for rapidly detecting, and optionally enumerating, a microorganism or a group of microorganisms of interest in a sample.

### BACKGROUND OF THE INVENTION

Microorganisms may contaminate any environment, any product which is designed for human and animal consumption or use such as food, beverages, medical devices, pharmaceutical or cosmetic products. The presence of these microorganisms can cause not only the deterioration or spoilage of the product, but also disease if consumed by or administered to a human or animal. To ensure safety of these products, microbiological tests are required to examine the risk of contamination under normal use conditions and thereby preventing poisoning or infection outbreaks. In particular, for medical devices, pharmaceutical or cosmetic industry, need of sterility is very important and testing the microbiological quality of products and raw materials is important along the whole supply chain as possible flaws of products can occur at every stage of production.

Of course, microorganisms may also infect humans or animals and detecting and/or quantifying microorganisms responsible of an infection is required in clinical diagnosis. For instance, diagnosis of urinary tract infection (UTI) may performed through quantitative urine culture. Traditionally, the presence of 1,000 or more bacteria/ml in urine has been considered to represent significant bacteriuria, indicating UTI.

For all these industries and clinical applications, the contamination thresholds and the methods used may vary. However, revealing the presence of relevant microorganisms at the earliest possible stage is always crucial.

Culture on a Petri dish, the Louis Pasteur's method, has been the most effective way of identifying and counting microorganisms since the 19th century. To date, this technique still remains the reference method in many industries to detect and count microorganisms. The method is simple: extracting the microorganisms from a sample, putting them on a dedicated Petri dish containing a suitable medium and counting colonies when they get visible to the eye. Though reliable, this method is long (up to several days according to the target microorganism).

In order to shorten the length of the assay, it was suggested to use indicators such as chromogenic substrates, fluorogenic substrates, or fluorescent or radio-labeled antibodies to detect smaller size microcolonies (e.g. International patent applications WO 96/14431, WO 2013/050598, WO 2008/118400). However, for most of these methods, the growth and indicator stages have to be segregated for optimal results: the growth stage for fast cell growth without harmful indicators therein that retard growth, and the indicator stage for dedicated coloring and identification that effectively allow smaller size microcolonies to be detected.

Over the years, many other attempts have been made to reduce the time required for the assay, in particular using culture independent approaches based on immunological methods, nucleic acid amplification methods, or flow cytometry methods using labeling by fluorescent antibodies or fluorogenic substrates. However, these methods remain expensive, have a not easy workflow, require highly skilled specialists and/or concentrated samples and, for most of them, it is very difficult to distinguish live and dead microorganisms.

Thus, there remains a need for a rapid, sensitive, user friendly and cost effective method for detecting and enumerating microorganisms in a sample.

### SUMMARY OF THE INVENTION

The inventors herein provide a method that fulfils this need.

In a first aspect, the present invention relates to a method for detecting or enumerating a microorganism or a group of microorganisms of interest comprising:
a) contacting, in a container, a sample suspected of containing said microorganism or group of microorganisms with a solid growth medium containing nutrients to support growth of said microorganism or group of microorganisms and with at least one fluorescent dye;
b) incubating said container under conditions and for a time sufficient to form microcolonies of said microorganism or group of microorganisms; and
c) detecting or enumerating microcolonies emitting a fluorescent signal of said at least one fluorescent dye,
thereby detecting or enumerating said microorganism or group of microorganisms contained in the sample.

Optionally, before step a), the microorganisms of the sample are concentrated on a membrane filter and in step a) said membrane and microorganisms are contacted with the solid growth medium and said at least one fluorescent dye.

Step a) may comprise
a1) contacting a sample suspected of containing said microorganism or group of microorganisms with at least one fluorescent dye, and
a2) contacting, in a container, said sample with a solid growth medium containing nutrients to support growth of said microorganism or group of microorganisms,
steps a1) and a2) being carried out simultaneously or sequentially, in any order.

Preferably, step a1) is carried out before step a2).

Optionally, before step a2), the microorganisms of the sample are concentrated on a membrane filter. In particular, the sample may be concentrated on a membrane filter before or after step a1), preferably after step a1). In particular, said at least one fluorescent dye may be filtered on the same membrane than the sample, before or after the filtration of the sample. The fluorescent dye(s) may be added to the sample prior to filtration/concentration on the membrane or may be filtered on the same membrane prior or after the filtration of the sample, preferably after the filtration of the sample. The membrane filter may be then placed on the surface of said growth medium.

Preferably, the membrane filter is made of mixed cellulose esters (MCE), polyvinylidene difluoride (PVDF), nitrocellulose, polytetrafluoroethylene, polycarbonate or nylon, more preferably is made of MCE or PVDF.

The solid growth medium may be selective for said microorganism or group of microorganisms.

Said at least one fluorescent dye may be selected from the group consisting of xanthene-based dyes, acridine dyes, anthracene-based dyes, coumarin-based dyes, cyanine-based dyes, dipyrromethene-based dyes, naphthalene-based dyes, pyrene-based dyes, squaraine-based dyes, squaraine rotaxane-based dyes and 4′,6-diamidino-2-phenylindole (DAPI), and combinations thereof. Preferably, said at least one fluorescent dye may be selected from the group consisting of MB660R, Acridine orange, DRAQ5, Cytrak Orange, Alexa Fluor 350, Pacific Blue, Cy5, preferably Sulfo-Cy5, Cy5.5, preferably Sulfo-Cy5.5, BODIPY 500/510, prodan, Alexa Fluor 405, Cascade Blue, Seta 650, Seta 375, Setau 647, SeTau 488, Alexa Fluor 488, ATTO 647 and DAPI, derivatives thereof, and combinations thereof. More particularly, said at least one fluorescent dye may be selected from the group consisting of MB660R-DBCO, MB660R-acid, Acridine orange, DRAQ5, Cytrak Orange, Alexa Fluor 350 NHS ester, Pacific Blue-NHS Ester, Sulfo-Cy5 acid, Sulfo-Cy5.5 acid, BODIPY 500/510, prodan, Alexa Fluor 405-DBCO, Cascade Blue, Seta 650-DBCO, Seta 375-NHS, SeTau 647-maleimide, SeTau 488-NHS, Alexa Fluor 488-acid, Alexa Fluor 488-DBCO, ATTO 647-acid and DAPI, derivatives thereof, and combinations thereof.

In some embodiments, said at least one fluorescent dye may be selected from the group consisting of anthracene-based dyes, coumarin-based dyes, cyanine-based dyes, dipyrromethene-based dyes, naphthalene-based dyes, pyrene-based dyes, squaraine-based dyes, squaraine rotaxane-based dyes, xanthene-based dyes and 4′,6-diamidino-2-phenylindole (DAPI), and combinations thereof. Preferably, said at least one fluorescent dye is selected from the group consisting of DRAQ5™, Alexa Fluor™ 350, Cy5™, preferably Sulfo-Cy5, BODIPY™ 500/510, prodan, Alexa Fluor™ 405, Seta™ 650, Setau™ 647, MB™ 660R, Alexa Fluor™ 488, ATTO™ 647 and DAPI, derivatives thereof, and combinations thereof. More preferably, said at least one fluorescent dye is DRAQ5™, Alexa Fluor™ 350-NHS ester, Sulfo-Cy5 acid, BODIPY™ 500/510, prodan, Alexa Fluor™ 405-DBCO, Seta™ 650-DBCO, SeTau™ 647-maleimide, MB™ 660R-acid, MB™ 660R-DBCO, Alexa Fluor™ 488-Acid, Alexa Fluor™ 488-DBCO, ATTO™ 647-acid and DAPI, and combinations thereof. Alternatively, said at least one fluorescent dye may be selected from the group consisting of DRAQ5™, Alexa Fluor™ 350, Pacific Blue™, Cy5™, preferably Sulfo-Cy5, BODIPY™ 500/510, prodan, Alexa Fluor™ 405, Cascade Blue™, Seta™ 650, Setau™ 647, SeTau 488, MB™ 660R, Alexa Fluor™ 488, ATTO™ 647 and DAPI, derivatives thereof, and combinations thereof. More preferably, said at least one fluorescent dye is DRAQ5™, Alexa Fluor™ 350-NHS ester, Pacific Blue™-NHS ester, Sulfo-Cy5 acid, BODIPY™ 500/510, prodan, Alexa Fluor™ 405-DBCO, Cascade Blue™, Seta™ 650-DBCO, SeTau 488-NHS, SeTau™ 647-maleimide, MB™ 660R-acid, MB™ 660R-DBCO, Alexa Fluor™ 488-Acid, Alexa Fluor™ 488-DBCO, ATTO™ 647-acid and DAPI, and combinations thereof.

Alternatively, said at least one fluorescent dye may be selected from the group consisting of xanthene-based dyes, anthracene-based dyes, coumarin-based dyes, cyanine-based dyes, dipyrromethene-based dyes, naphthalene-based dyes, pyrene-based dyes, squaraine-based dyes and squaraine rotaxane-based dyes, and combinations thereof. In particular, said at least one fluorescent dye may be selected from the group consisting of MB660R, DRAQ5, Cytrak Orange, Alexa Fluor 350, Pacific Blue, Cy5, preferably Sulfo-Cy5, Cy5.5, preferably Sulfo-Cy5.5, BODIPY 500/510, prodan, Alexa Fluor 405, Cascade Blue, Seta 650, Seta 375, Setau 647, SeTau 488, Alexa Fluor 488 and ATTO 647, derivatives thereof, and combinations thereof. More particularly, said at least one fluorescent dye may be selected from the group consisting of MB660R-DBCO, MB660R-acid, DRAQ5, Cytrak Orange, Alexa Fluor 350 NHS ester, Pacific Blue-NHS Ester, Sulfo-Cy5 acid, Sulfo-Cy5.5 acid, BODIPY 500/510, prodan, Alexa Fluor 405-DBCO, Cascade Blue, Seta 650-DBCO, Seta 375-NHS, SeTau 647-maleimide, SeTau 488-NHS, Alexa Fluor 488-acid, Alexa Fluor 488-DBCO and ATTO 647-acid, derivatives thereof, and combinations thereof.

In particular, the microorganism or group of microorganisms of interest may
belong to thermophilic acidophilic bacteria, and is preferably selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius and Alicyclobacillus contaminans, and combinations thereof; and/or
belong to acetic acid bacteria, and is preferably selected from bacteria belonging to the genera Acetobacter, Gluconobacter, Gluconacetobacter and Asaia, and combinations thereof; and/or
belong to lactic acid bacteria, and is preferably selected from bacteria belonging to the genera Lactobacillus and Weissella, and combinations thereof; and/or
belong to of anaerobic bacteria, and is preferably selected from Clostridium and Cutibacterium bacteria, and combinations thereof, and/or
belong to aerobic mesophilic bacteria, and is preferably selected from the genera Methylobacterium, Pseudomonas, Bacillus, Escherichia, Staphylococcus, Acinetobacter, Cronobacter, Klebsiella, Salmonella, Enterococcus, Listeria, Shigella, Kocuria and Burkholderia, and combinations thereof, and/or
belong to Gram negative bacteria, and is preferably selected from the genera Escherichia and Pseudomonas, and combinations thereof, and/or
belong to the group of yeasts and molds, and is preferably selected from the genera Candida, Zygosaccharomyces, Aspergillus, Saccharomyces, Geotrichum and Penicillium, and combinations thereof, and/or
belong to the group of heterotrophic bacteria, preferably selected from bacteria belonging to the genera Acinetobacter, Aeromonas, Brevundimonas, Burkholderia, Citrobacter, Edwardsiella, Enterobacter, Escherichia, Ochrobactrum, Klebsiella, Methylobacterium, Moraxella, Pantoea, Proteus, Pseudomonas, Ralstonia, Salmonella, Serratia, Shigella, Sphingomonas, Stenotrophomonas, Vibrio, Yersinia, Bacillus, Enterococcus, Micrococcus, Staphylococcus, and combinations thereof.

Preferably, in the method of the invention,
the microorganism or group of microorganisms of interest belongs to or comprises a microorganism belonging to the group of thermophilic acidophilic bacteria, preferably selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius and Alicyclobacillus contaminans, and combinations thereof, and said at least one fluorescent dye is selected from the group consisting of xanthene-based dyes, preferably selected from the group consisting of Alexa Fluor 488, MB660R and ATTO647, and derivatives and combinations thereof, more preferably selected from the group consisting of Alexa Fluor 488-Acid, Alexa Fluor 488-DBCO, MB660R-acid, MB660R-DBCO and ATTO647-acid, and combinations thereof. Preferably, said at least one fluorescent dye is selected from the group consisting of xanthene-based dyes, preferably from the group consisting of MB660R and derivatives, more preferably from the group consisting of MB660R-Acid and MB660R-DBCO, even more preferably is MB660R-DBCO; and/or
the microorganism or group of microorganisms of interest belongs to or comprises a microorganism belonging to the group of acetic acid bacteria (AAB), preferably selected from bacteria belonging to the genera Acetobacter, Gluconobacter, Gluconacetobacter and Asaia, and combinations thereof, more preferably selected from the group consisting of Acetobacter aceti, Gluconoacetobacter liquefaciens, Gluconobacter oxydans and Asaia siamensis, and combinations thereof, and said at least one fluorescent dye is selected from the group consisting of xanthene-based dyes and DAPI, and combinations thereof, preferably selected from the group consisting of Alexa Fluor 488, MB660R, ATTO647 and DAPI, and derivatives and combinations thereof, more preferably selected from the group consisting of Alexa Fluor 488-Acid, Alexa Fluor 488-DBCO, MB660R-acid, MB660R-DBCO, ATTO647-acid and DAPI, and combinations thereof. Preferably, said at least one fluorescent dye is selected from the group consisting of xanthene-based dyes, preferably selected from the group consisting of Alexa Fluor 488, MB660R and ATTO647, and derivatives and combinations thereof, more preferably selected from the group consisting of Alexa Fluor 488-acid, Alexa Fluor 488-DBCO, MB660R-acid, MB660R-DBCO and ATTO647-acid, and combinations thereof. More preferably, said at least one fluorescent dye is selected from the group consisting of MB660R and ATTO647, and derivatives and combinations thereof, preferably selected from the group consisting of MB660R-acid, MB660R-DBCO and ATTO647-acid, and combinations thereof, more preferably selected from the group consisting of MB660R-acid and ATTO647-acid, and combinations thereof; and/or
the microorganism or group of microorganisms of interest belongs to or comprises a microorganism belonging to the group of lactic acid bacteria (LAB), preferably selected from bacteria belonging to the genera Lactobacillus and Weissella, and combinations thereof, and more preferably selected from the group consisting of Lactobacillus casei, Lactobacillus plantarum and Weissella confusa, and combinations thereof, and said at least one fluorescent dye is selected from the group consisting of xanthene-based dyes and DAPI, and combinations thereof, preferably selected from the group consisting of Alexa Fluor 488, MB660R, ATTO647 and DAPI, and derivatives and combinations thereof, more preferably selected from the group consisting of Alexa Fluor 488-Acid, Alexa Fluor 488-DBCO, MB660R-acid, MB660R-DBCO and ATTO647-acid and combinations thereof. Preferably, said at least one fluorescent dye is selected from the group consisting of Alexa Fluor 488, MB660R and DAPI, and derivatives and combinations thereof, in particular selected from the group consisting of Alexa Fluor 488-acid, MB660R-acid and DAPI, and combinations thereof, preferably selected from the group consisting of xanthene-based dyes, more preferably selected from the group consisting of Alexa Fluor 488-acid and MB660R-acid and combinations thereof, and even more preferably is MB660R-acid; and/or
the microorganism or group of microorganisms of interest belongs to or comprises a microorganism belonging to the group of anaerobic bacteria, preferably selected from Clostridium and Cutibacterium bacteria, and combinations thereof, more preferably selected from the group consisting of Clostridium sporogenes and Cutibacterium acnes, and combinations thereof, and said at least one fluorescent dye is selected from the group consisting of xanthene-based dyes, cyanine-based dyes and DAPI, and combinations thereof, preferably selected from the group consisting of MB660R, Cy5™, preferably sulfo-Cy5, and DAPI, and derivatives and combinations thereof, more preferably from the group consisting of MB660R-DBCO, sulfo-Cy5 acid and DAPI, and combinations thereof. Preferably, said at least one fluorescent dye is selected from the group consisting of xanthene-based dyes and cyanine-based dyes, preferably selected from the group consisting of MB660R and Cy5, preferably sulfo-Cy5, and derivatives and combinations thereof, more preferably from the group consisting of MB660R-DBCO and sulfo-Cy5 acid, and combinations thereof. More preferably, said at least one fluorescent dye comprises MB660R-DBCO and sulfo-Cy5 acid; and/or
the microorganism or group of microorganisms of interest belongs to or comprises a microorganism belonging to the group of aerobic mesophilic bacteria (AMB), preferably selected from bacteria belonging to the genera Methylobacterium, Pseudomonas, Bacillus, Escherichia, Staphylococcus, Acinetobacter, Cronobacter, Klebsiella, Salmonella, Enterococcus, Listeria, Shigella, Kocuria and Burkholderia, and combinations thereof, more preferably selected from the group consisting of Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Listeria grayi, Shigella sonnei, Kocuria rhizophila and Burkholderia cepacia, and combinations thereof, and said at least one fluorescent dye is selected from the group consisting of xanthene-based dyes, anthracene-based dyes, coumarin-based dyes, cyanine-based dyes, dipyrromethene-based dyes, naphthalene-based dyes, pyrene-based dyes, squaraine-based dyes, squaraine rotaxane-based dyes and DAPI, and combinations thereof, preferably selected from the group consisting of DRAQ5™, Alexa Fluor™ 350, Pacific Blue™, Cy5™, preferably Sulfo-Cy5, BODIPY™ dyes, preferably BODIPY 500/510, prodan, Alexa Fluor™ 405, Cascade Blue™, Seta dyes, preferably Seta 650, SeTau dyes, preferably SeTau 647 and SeTau 488, Alexa Fluor 488, MB660R, ATTO647 and DAPI, and derivatives and combinations thereof, more preferably selected from the group consisting of DRAQ5™, Alexa Fluor™ 350-NHS ester, Pacific Blue™-NHS ester, Sulfo-Cy5-acid, BODIPY 500/510, prodan, Alexa Fluor™ 405-DBCO, Cascade Blue™, Seta 650-DBCO, SeTau 488-NHS, SeTau 647-Maleimide, Alexa Fluor 488-Acid, Alexa Fluor 488-DBCO, MB660R-acid, MB660R-DBCO, ATTO647-acid and DAPI, and combinations thereof; and/or
the microorganism or group of microorganisms of interest belongs to or comprises a microorganism belonging to the group of aerobic mesophilic bacteria (AMB), preferably selected from bacteria belonging to the genera Methylobacterium, Pseudomonas, Bacillus, Escherichia, Staphylococcus, Acinetobacter, Cronobacter, Klebsiella, Salmonella, Enterococcus, Shigella, Kocuria and Burkholderia, and combinations thereof, more preferably selected from the group consisting of Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Shigella sonnei, Kocuria rhizophila and Burkholderia cepacia, and combinations thereof, and said at least one fluorescent dye is selected from the group consisting of xanthene-based dyes, acridine dyes, anthracene-based dyes, coumarin-based dyes, cyanine-based dyes, dipyrromethene-based dyes, naphthalene-based dyes, pyrene-based dyes, squaraine-based dyes, squaraine rotaxane-based dyes and DAPI, and combinations thereof, preferably selected from the group consisting of Acridine orange, DRAQ5, Cytrak orange, Alexa Fluor 350, Pacific Blue, Cy5, preferably Sulfo-Cy5, BODIPY dyes, preferably BODIPY 500/510, prodan, Alexa Fluor 405, Cascade Blue, Seta dyes, preferably Seta 650 or Seta 375, SeTau dyes, preferably SeTau 647 or SeTau 488, Alexa Fluor 488, MB660R, ATTO647 and DAPI, and derivatives and combinations thereof, more preferably selected from the group consisting of Acridine orange, DRAQ5, Cytrak orange, Alexa Fluor 350-NHS ester, Pacific Blue-NHS Ester, Sulfo-Cy5-acid, BODIPY500/510, prodan, Alexa Fluor 405-DBCO, Cascade Blue, Seta 650-DBCO, Seta 375-NHS, SeTau 647-Maleimide, SeTau 488-NHS, Alexa Fluor 488-acid, Alexa Fluor 488-DBCO, MB660R-acid, MB660R-DBCO, ATTO647-acid and DAPI, and combinations thereof. Preferably, said at least one fluorescent dye is selected from the group consisting of xanthene-based dyes, anthracene-based dyes, coumarin-based dyes, cyanine-based dyes, dipyrromethene-based dyes, naphthalene-based dyes, pyrene-based dyes, squaraine-based dyes and squaraine rotaxane-based dyes, and combinations thereof, preferably selected from the group consisting of DRAQ5, Cytrak orange, Alexa Fluor 350, Pacific Blue, Cy5, preferably Sulfo-Cy5, BODIPY dyes, preferably BODIPY 500/510, prodan, Alexa Fluor 405, Cascade Blue, Seta dyes, preferably Seta 650 or Seta 375, SeTau dyes, preferably SeTau 647 or SeTau 488, Alexa Fluor 488, MB660R and ATTO647, and derivatives and combinations thereof, more preferably selected from the group consisting of DRAQ5, Cytrak orange, Alexa Fluor 350-NHS ester, Pacific Blue-NHS Ester, Sulfo-Cy5-acid, BODIPY 500/510, prodan, Alexa Fluor 405-DBCO, Cascade Blue, Seta 650-DBCO, Seta 375-NHS, SeTau 647-Maleimide, SeTau 488-NHS, Alexa Fluor 488-acid, Alexa Fluor 488-DBCO, MB660R-acid, MB660R-DBCO and ATTO647-acid, and combinations thereof. More preferably, said at least one fluorescent dye is selected from the group consisting of xanthene-based dyes, preferably selected from the group consisting of MB660R and derivatives, and combinations thereof, optionally in combination with a fluorescent dye selected from the group consisting of cyanine based dyes, preferably Cy5, in particular Sulfo-Cy5, more preferably is MB660R-DBCO, optionally in combination with Sulfo-Cy5-Acid; and/or
the microorganism or group of microorganisms of interest belongs to or comprises a microorganism belonging to the group of Gram negative bacteria, preferably selected from bacteria belonging to the genera Escherichia, Proteus and Pseudomonas, and combinations thereof, more preferably selected from Escherichia coli, Proteus mirabilis and Pseudomonas aeruginosa, and combinations thereof, and said at least one fluorescent dye may be selected from the group consisting of xanthene-based dyes, preferably rhodamine dyes, cyanine-based dyes and DAPI and combinations thereof, preferably selected from the group consisting of Cy5, preferably Sulfo-Cy5, MB660R and DAPI, and derivatives and combinations thereof, more preferably selected from the group consisting of Sulfo-Cy5-acid, MB660R-DBCO and DAPI, and combinations thereof. Preferably, said at least one fluorescent dye is selected from the group consisting of xanthene-based dyes, cyanine-based dyes and DAPI, and combinations thereof, preferably selected from the group consisting of Cy5™, preferably Sulfo-Cy5, MB660R, DAPI and derivatives and combinations thereof, more preferably selected from the group consisting of Sulfo-Cy5-acid, MB660R-DBCO and DAPI, and combinations thereof; and/or
the microorganism or group of microorganisms of interest belongs to or comprises a microorganism belonging to the group of Gram negative bacteria, preferably selected from bacteria belonging to the genera Escherichia and Pseudomonas, and combinations thereof, more preferably selected from Escherichia coli and Pseudomonas aeruginosa, and combinations thereof, and said at least one fluorescent dye is selected from the group consisting of xanthene-based dyes, preferably rhodamine dyes, pyrene-based dyes, coumarin-based dyes, cyanine-based dyes and DAPI, and combinations thereof, preferably selected from the group consisting of Cy5, preferably Sulfo-Cy5, Alexa Fluor 350, Alexa Fluor 405, MB660R and DAPI, and derivatives and combinations thereof, more preferably selected from the group consisting of Sulfo-Cy5-acid, Alexa Fluor 350-NHS ester, Alexa Fluor 405-DBCO, MB660R-DBCO and DAPI, and combinations thereof. Preferably, said at least one fluorescent dye is selected from the group consisting of xanthene-based dyes, preferably rhodamine dyes, pyrene-based dyes, coumarin-based dyes and cyanine-based dyes, and combinations thereof, preferably selected from the group consisting of Cy5, preferably Sulfo-Cy5, Alexa Fluor 350, Alexa Fluor 405 and MB660R, and derivatives and combinations thereof, more preferably selected from the group consisting of Sulfo-Cy5-acid, Alexa Fluor 350-NHS ester, Alexa Fluor 405-DBCO and MB660R-DBCO, and combinations thereof. More preferably, said at least one fluorescent dye is selected from the group consisting of pyrene-based dyes, coumarin-based dyes, and combinations thereof, preferably selected from the group consisting of Alexa Fluor 350 and Alexa Fluor 405, and derivatives and combinations thereof, more preferably selected from the group consisting of Alexa Fluor 350-NHS ester, Alexa Fluor 405-DBCO, and combinations thereof. Even more preferably, said at least one fluorescent dye is a combination of Alexa Fluor 350-NHS ester and Alexa Fluor 405-DBCO; and/or
the microorganism or group of microorganisms of interest belongs to or comprises a microorganism belonging to the group of Gram positive bacteria, preferably selected from bacteria belonging to the genera Staphylococcus and Enterococcus, and combination thereof, more preferably selected from Staphylococcus aureus and Enterococcus faecalis, and combination thereof, and said at least one fluorescent dye is selected from the group consisting of xanthene-based dyes, cyanine-based dyes and DAPI, and combinations thereof, preferably selected from the group consisting of Cy5™, preferably Sulfo-Cy5, MB660R, DAPI and derivatives and combinations thereof, more preferably selected from the group consisting of Sulfo-Cy5-acid, MB660R-DBCO and DAPI, and combinations thereof. Preferably, said at least one fluorescent dye is selected from the group consisting of xanthene-based dyes and cyanine-based dyes, and combinations thereof, preferably selected from the group consisting of Cy5, preferably Sulfo-Cy5, and MB660R, and derivatives and combinations thereof, more preferably selected from the group consisting of Sulfo-Cy5-acid and MB660R-DBCO, and combinations thereof; and/or
the microorganism or group of microorganisms of interest belongs to or comprises a microorganism belonging to the group of heterotrophic bacteria, preferably selected from bacteria belonging to the genera Acinetobacter, Aeromonas, Brevundimonas, Burkholderia, Citrobacter, Edwardsiella, Enterobacter, Escherichia, Ochrobactrum, Klebsiella, Methylobacterium, Moraxella, Pantoea, Proteus, Pseudomonas, Ralstonia, Salmonella, Serratia, Shigella, Sphingomonas, Stenotrophomonas, Vibrio, Yersinia, Bacillus, Enterococcus, Micrococcus, Staphylococcus, and combinations thereof, more preferably selected from Acinetobacter baumanii, Aeromonas hydrophila, Brevundimonas diminuta, Burkholderia cepacia, Citrobacter freundii, Edwardsiella tarda, Enterobacter aerogenes, Escherichia coli, Ochrobactrum anthropic, Klebsiella pneumoniae, Methylobacterium extorquens, Moraxella osloensis, Pantoea agglomerans, Proteus mirabilis, Pseudomonas aeruginosa, Pseudomonas fluorescens, Ralstonia pickettii, Salmonella typhimurium, Serratia marcescens, Shigella sonnei, Sphingomonas paucimobilis, Stenotrophomonas maltophilia, Vibrio parahaemolyticus, Yersinia enterocolitica, Bacillus subtilis, Enterococcus faecalis, Micrococcus luteus (Kocuria rhizophila), Staphylococcus aureus, and combinations thereof, and said at least one fluorescent dye is selected from the group consisting of coumarin-based dyes, cyanine-based dyes, pyrene-based dyes and xanthene-based dyes, and combinations thereof, preferably selected from the group consisting of Alexa Fluor™ 350, Cy5™, preferably Sulfo-Cy5, Alexa Fluor™ 405, and MB660R, and derivatives and combinations thereof, more preferably selected from the group consisting of Alexa Fluor™ 350-NHS ester, Sulfo-Cy5-acid, Alexa Fluor™ 405-DBCO and MB660R-DBCO, and combinations thereof, and even more preferably selected from the group consisting of (i) a mix of MB660R and Sulfo-Cy5, preferably MB660R-DBCO and Sulfo-Cy5-Acid and (ii) a mix of Alexa Fluor™ 350 and Alexa Fluor™ 405, preferably Alexa Fluor™ 350-NHS ester and Alexa Fluor™ 405-DBCO; and/or
the microorganism or group of microorganisms of interest belongs to or comprises a microorganism belonging to the group of heterotrophic bacteria, preferably selected from bacteria belonging to the genera Acinetobacter, Aeromonas, Brevundimonas, Burkholderia, Citrobacter, Edwardsiella, Enterobacter, Escherichia, Ochrobactrum, Klebsiella, Methylobacterium, Moraxella, Pantoea, Proteus, Pseudomonas, Ralstonia, Salmonella, Serratia, Shigella, Sphingomonas, Stenotrophomonas, Yersinia, Bacillus, Enterococcus, Micrococcus, Staphylococcus, and combinations thereof, more preferably selected from Acinetobacter baumanii, Aeromonas hydrophila, Brevundimonas diminuta, Burkholderia cepacia, Citrobacter freundii, Edwardsiella tarda, Enterobacter aerogenes, Escherichia coli, Ochrobactrum anthropic, Klebsiella pneumoniae, Methylobacterium extorquens, Moraxella osloensis, Pantoea agglomerans, Proteus mirabilis, Pseudomonas aeruginosa, Pseudomonas fluorescens, Ralstonia pickettii, Salmonella typhimurium, Serratia marcescens, Shigella sonnei, Sphingomonas paucimobilis, Stenotrophomonas maltophilia, Yersinia enterocolitica, Bacillus subtilis, Enterococcus faecalis, Micrococcus luteus (Kocuria rhizophila), Staphylococcus aureus, and combinations thereof, and said at least one fluorescent dye is selected from the group consisting of acridine dyes, xanthene-based dyes, anthracene-based dyes, coumarin-based dyes, cyanine-based dyes, dipyrromethene-based dyes, naphthalene-based dyes, pyrene-based dyes, squaraine-based dyes, squaraine rotaxane-based dyes and DAPI, and combinations thereof, preferably selected from the group consisting of acridine orange, DRAQ5, Cytrak orange, Alexa Fluor 350, Pacific Blue, Cy5, preferably Sulfo-Cy5, Cy5.5, preferably Sulfo-Cy5.5, BODIPY dyes, preferably BODIPY 500/510, prodan, Alexa Fluor 405, Cascade Blue, Seta dyes, preferably Seta 650 or Seta 375, SeTau dyes, preferably SeTau 647 or SeTau 488, Alexa Fluor 488, MB660R, ATTO647 and DAPI, and derivatives and combinations thereof, more preferably selected from the group consisting of acridine orange, DRAQ5, Cytrak orange, Alexa Fluor 350-NHS ester, Pacific Blue-NHS Ester, Sulfo-Cy5-acid, Sulfo-Cy5.5-acid, BODIPY 500/510, prodan, Alexa Fluor 405-DBCO, Cascade Blue, Seta 650-DBCO, Seta 375-NHS, SeTau 647-Maleimide, SeTau 488-NHS, Alexa Fluor 488-acid, Alexa Fluor 488-DBCO, MB660R-acid, MB660R-DBCO, ATTO647-acid and DAPI, and combinations thereof. Preferably, said at least one fluorescent dye is selected from the group consisting of xanthene-based dyes, anthracene-based dyes, coumarin-based dyes, cyanine-based dyes, dipyrromethene-based dyes, naphthalene-based dyes, pyrene-based dyes, squaraine-based dyes and squaraine rotaxane-based dyes, and combinations thereof, preferably selected from the group consisting of DRAQ5, Cytrak orange, Alexa Fluor 350, Pacific Blue, Cy5, preferably Sulfo-Cy5, Cy5.5, preferably Sulfo-Cy5.5, BODIPY dyes, preferably BODIPY 500/510, prodan, Alexa Fluor 405, Cascade Blue, Seta dyes, preferably Seta 650 or Seta 375, SeTau dyes, preferably SeTau 647 or SeTau 488, Alexa Fluor 488, MB660R and ATTO647, and derivatives and combinations thereof, more preferably selected from the group consisting of DRAQ5, Cytrak orange, Alexa Fluor 350-NHS ester, Pacific Blue-NHS Ester, Sulfo-Cy5-acid, Sulfo-Cy5.5-acid, BODIPY 500/510, prodan, Alexa Fluor 405-DBCO, Cascade Blue, Seta 650-DBCO, Seta 375-NHS, SeTau 647-Maleimide, SeTau 488-NHS, Alexa Fluor 488-acid, Alexa Fluor 488-DBCO, MB660R-acid, MB660R-DBCO and ATTO647-acid, and combinations thereof; and/or
the microorganism or group of microorganisms of interest belongs to or comprises a microorganism belonging to the group of yeasts and molds, preferably selected from fungi belonging to the genera Candida, Zygosaccharomyces, Aspergillus, Saccharomyces, Geotrichum and Penicillium, and combinations thereof, more preferably selected from the group consisting of Candida albicans, Zygosaccharomyces baili, Aspergillus brasiliensis, Saccharomyces cerevisiae, Geotrichum candidum, Penicillium variotii, Penicillium chrysogenum, and combinations thereof, and said at least one fluorescent dye is selected from the group consisting of acridine dyes, xanthene-based dyes, preferably selected from the group consisting of rhodamine dyes, fluorescein dyes and carbopyronine-based dyes, coumarin-based dyes, cyanine-based dyes, dipyrromethene-based dyes, naphthalene-based dyes, pyrene-based dyes, squaraine-based dyes, squaraine rotaxane-based dyes and DAPI, and combinations thereof, preferably selected from the group consisting of Acridine orange, Alexa Fluor 350, Pacific Blue, Cy5, preferably Sulfo-Cy5, prodan, Alexa Fluor 405, Cascade Blue, BODIPY 500/510, Seta dyes, preferably Seta 650, SeTau dyes, preferably SeTau 647 or SeTau 488, Alexa Fluor 488, MB660R and DAPI, and derivatives and combinations thereof, more preferably selected from the group consisting of Acridine orange, Alexa Fluor 350-NHS ester, Pacific Blue-NHS Ester, Sulfo-Cy5-acid, prodan, Alexa Fluor 405-DBCO, Cascade Blue, BODIPY 500/510, Seta 650-DBCO, SeTau 647-Maleimide, SeTau 488-NHS, Alexa Fluor 488-acid, MB660R-acid, MB660R-DBCO and DAPI, and combinations thereof. Preferably, said at least one fluorescent dye is selected from the group consisting of xanthene-based dyes, coumarin-based dyes, cyanine-based dyes, naphthalene-based dyes, pyrene-based dyes, squaraine-based dyes, squaraine rotaxane-based dyes and DAPI, and combinations thereof, preferably selected from the group consisting of Alexa Fluor™ 350, Pacific Blue™, Cy5™, preferably Sulfo-Cy5, prodan, Alexa Fluor™ 405, Cascade Blue™, Seta dyes, preferably Seta 650, SeTau dyes, preferably SeTau 647 and SeTau 488, Alexa Fluor 488, MB660R, ATTO647 and DAPI, and derivatives and combinations thereof, more preferably selected from the group consisting of Alexa Fluor™ 350-NHS ester, Pacific Blue™-NHS ester, Sulfo-Cy5-acid, prodan, Alexa Fluor™ 405-DBCO, Cascade Blue™, Seta 650-DBCO, SeTau 488-NHS, SeTau 647-Maleimide, Alexa Fluor 488-Acid, MB660R-acid, MB660R-DBCO, ATTO647-acid and DAPI, and combinations thereof. More preferably, said at least one fluorescent dye is selected from the group consisting of xanthene-based dyes, preferably selected from the group consisting of MB660R and derivatives, and combinations thereof, preferably is MB660R-DBCO; and/or
the microorganism or group of microorganisms of interest comprises one or several bacteria, preferably selected from the groups of thermophilic acidophilic bacteria (TAB), acetic acid bacteria (AAB), lactic acid bacteria (LAB), anaerobic bacteria, aerobic mesophilic bacteria, Gram negative bacteria and Gram positive bacteria, and combinations thereof, and one or several microscopic fungi, preferably selected from yeasts and molds, and said at least one fluorescent dye is selected from the group consisting of xanthene-based dyes, preferably selected from the group consisting of rhodamine dyes, fluorescein dyes and carbopyronine-based dyes, coumarin-based dyes, cyanine-based dyes, pyrene-based dyes, squaraine-based dyes and squaraine rotaxane-based dyes, and combinations thereof, preferably selected from the group consisting of Alexa Fluor™ 350, Pacific Blue™, Cy5™, preferably Sulfo-Cy5, Alexa Fluor™ 405, Cascade Blue™, Seta dyes, preferably Seta 650, SeTau dyes, preferably SeTau 647 and SeTau 488, Alexa Fluor 488 and MB660R, and derivatives and combinations thereof, more preferably selected from the group consisting of Alexa Fluor™ 350-NHS ester, Pacific Blue™-NHS ester, Sulfo-Cy5-acid, Alexa Fluor™ 405-DBCO, Cascade Blue™, Seta 650-DBCO, SeTau 488-NHS, SeTau 647-Maleimide, Alexa Fluor 488-Acid, MB660R-acid and MB660R-DBCO, and combinations thereof, and even more preferably selected from the group consisting of Sulfo-Cy5-acid and MB660R-DBCO, and combinations thereof.

In some embodiments, the microorganism or group of microorganisms of interest comprises one or several bacteria, preferably selected from the groups of thermophilic acidophilic bacteria (TAB), acetic acid bacteria (AAB), lactic acid bacteria (LAB), anaerobic bacteria, aerobic mesophilic bacteria, Gram negative bacteria and Gram positive bacteria, heterotrophic bacteria, and combinations thereof, and one or several microscopic fungi, preferably selected from yeasts and molds, and said at least one fluorescent dye is selected from the group consisting of xanthene-based dyes, preferably selected from the group consisting of rhodamine dyes, fluorescein dyes and carbopyronine-based dyes, coumarin-based dyes, cyanine-based dyes, pyrene-based dyes, squaraine-based dyes and squaraine rotaxane-based dyes, and combinations thereof, preferably selected from the group consisting of Alexa Fluor 350, Pacific blue, Cy5, preferably Sulfo-Cy5, Alexa Fluor 405, Cascade Blue™, Seta dyes, preferably Seta 650 or Seta 375, SeTau dyes, preferably SeTau 647 and SeTau 488, Alexa Fluor 488 and MB660R, and derivatives and combinations thereof, more preferably selected from the group consisting of Alexa Fluor 350-NHS ester, Pacific Blue™-NHS ester, Sulfo-Cy5-acid, Alexa Fluor 405-DBCO, Cascade blue, Seta 650-DBCO, Seta 375-NHS, SeTau 488-NHS, SeTau 647-Maleimide, Alexa Fluor 488-Acid, MB660R-acid and MB660R-DBCO, and combinations thereof, and even more preferably selected from the group consisting of Sulfo-Cy5-acid and MB660R-DBCO, and combinations thereof.

In particular, the microorganism or group of microorganisms of interest may belong to or comprises a microorganism belonging to the group of aerobic mesophilic bacteria (AMB), preferably selected from bacteria belonging to the genera Methylobacterium, Pseudomonas, Bacillus, Escherichia, Staphylococcus, Acinetobacter, Cronobacter, Klebsiella, Salmonella, Enterococcus, Listeria, Shigella, Kocuria and Burkholderia, and combinations thereof, more preferably selected from the group consisting of Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Listeria grayi, Shigella sonnei, Kocuria rhizophila and Burkholderia cepacia, and combinations thereof, and said at least one fluorescent dye may be selected from the group consisting of xanthene-based dyes, anthracene-based dyes, coumarin-based dyes, cyanine-based dyes, dipyrromethene-based dyes, naphthalene-based dyes, pyrene-based dyes, squaraine-based dyes, squaraine rotaxane-based dyes and DAPI, and combinations thereof, preferably selected from the group consisting of DRAQ5™, Alexa Fluor™ 350, Cy5™, preferably Sulfo-Cy5, BODIPY™ dyes, preferably BODIPY 500/510, prodan, Alexa Fluor™ 405, Seta dyes, preferably Seta 650, SeTau dyes, preferably SeTau 647, Alexa Fluor 488, MB660R, ATTO647 and DAPI, and derivatives and combinations thereof, more preferably selected from the group consisting of DRAQ5™, Alexa Fluor™ 350-NHS ester, Sulfo-Cy5-acid, BODIPY 500/510, prodan, Alexa Fluor™ 405-DBCO, Seta 650-DBCO, SeTau 647-Maleimide, Alexa Fluor 488-Acid, Alexa Fluor 488-DBCO, MB660R-acid, MB660R-DBCO, ATTO647-acid and DAPI, and combinations thereof.

In particular, the microorganism or group of microorganisms of interest may belong to or may comprise a microorganism belonging to the group of yeasts and molds, preferably selected from fungi belonging to the genera Candida, Zygosaccharomyces, Aspergillus, Saccharomyces, Geotrichum and Penicillium, and combinations thereof, more preferably selected from the group consisting of Candida albicans, Zygosaccharomyces baili, Aspergillus brasiliensis, Saccharomyces cerevisiae, Geotrichum candidum, Penicillium variotii, Penicillium chrysogenum, and combinations thereof, and said at least one fluorescent dye may be selected from the group consisting of xanthene-based dyes, coumarin-based dyes, cyanine-based dyes, naphthalene-based dyes, pyrene-based dyes, squaraine-based dyes, squaraine rotaxane-based dyes and DAPI, and combinations thereof, preferably selected from the group consisting of Alexa Fluor™ 350, Cy5™, preferably Sulfo-Cy5, prodan, Alexa Fluor™ 405, Seta dyes, SeTau dyes, Alexa Fluor 488, MB660R, ATTO647 and DAPI, and derivatives and combinations thereof, more preferably selected from the group consisting of Alexa Fluor™ 350-NHS ester, Sulfo-Cy5-acid, prodan, Alexa Fluor™ 405-DBCO, Seta 650-DBCO, SeTau 647-Maleimide, Alexa Fluor 488-Acid, MB660R-acid, MB660R-DBCO, ATTO647-acid and DAPI, and combinations thereof.

In particular, the microorganism or group of microorganisms of interest may belong to or may comprise a microorganism belonging to the group of Gram negative bacteria, preferably selected from bacteria belonging to the genera Escherichia and Pseudomonas, and combinations thereof, more preferably selected from Escherichia coli and Pseudomonas aeruginosa, and combinations thereof, and said at least one fluorescent dye may be selected from the group consisting of xanthene-based dyes and cyanine-based dyes, and combinations thereof, preferably selected from the group consisting of Cy5™, preferably Sulfo-Cy5, MB660R, and derivatives and combinations thereof, more preferably selected from the group consisting of Sulfo-Cy5-acid and MB660R-DBCO, and combinations thereof.

In particular, the microorganism or group of microorganisms of interest may comprise one or several bacteria, preferably selected from the groups of thermophilic acidophilic bacteria (TAB), acetic acid bacteria (AAB), lactic acid bacteria (LAB), anaerobic bacteria, aerobic mesophilic bacteria, Gram negative bacteria and Gram positive bacteria, and combinations thereof, and one or several microscopic fungi, preferably selected from yeasts and molds, and said at least one fluorescent dye may be selected from the group consisting of xanthene-based dyes, preferably selected from the group consisting of rhodamine dyes, fluorescein dyes and carbopyronine-based dyes, coumarin-based dyes, cyanine-based dyes, pyrene-based dyes, squaraine-based dyes and squaraine rotaxane-based dyes, and combinations thereof, preferably selected from the group consisting of Alexa Fluor™ 350, Cy5™, preferably Sulfo-Cy5, Alexa Fluor™ 405, Seta dyes, preferably Seta 650, SeTau dyes, preferably SeTau 647, Alexa Fluor 488 and MB660R, and derivatives and combinations thereof, more preferably selected from the group consisting of Alexa Fluor™ 350-NHS ester, Sulfo-Cy5-acid, Alexa Fluor™ 405-DBCO, Seta 650-DBCO, SeTau 647-Maleimide, Alexa Fluor 488-Acid, MB660R-acid and MB660R-DBCO, and combinations thereof, and even more preferably selected from the group consisting of Sulfo-Cy5-acid and MB660R-DBCO, and combinations thereof.

In particular, the microorganism or group of microorganisms of interest may belong to thermophilic acidophilic bacteria and said at least one fluorescent dye may comprise a dye selected from the group of xanthene-based dyes, preferably selected from MB™ 660R and derivatives thereof, more preferably MB™ 660R DBCO.

In particular, the microorganism or group of microorganisms of interest may belong to or comprise a microorganism selected from the group consisting of Aspergillus brasiliensis, Candida albicans and Escherichia coli, and combinations thereof, and said at least one fluorescent dye is selected from the group consisting of Bodipy 500/510; Alexa Fluor 350 or a derivative thereof, preferably Alexa Fluor 350-NHS ester; Pacific blue or a derivative thereof, preferably Pacific blue-NHS ester; prodan; Alexa Fluor 405 or a derivative thereof, preferably Alexa Fluor 405-DBCO; Cascade Blue; Seta 650 or a derivative thereof, preferably Seta 650-DBCO; Setau 488 or a derivative thereof, preferably Setau 488-NHS; Setau 647 or a derivative thereof, preferably Setau 647-maleimide, and any combination thereof.

In embodiments wherein the microorganisms of the sample are concentrated on a membrane filter before step a) or step a2), the membrane filter may be removed after step b) and before in step c), from the solid growth medium and microcolonies thereon may be detected and/or enumerated in step c). Alternatively, the membrane filter may be kept of the solid growth medium to perform step c).

Preferably, in step c) microcolonies emitting a fluorescent signal corresponding to said at least one fluorescent dye are detected and/or enumerated using a fluorescence microscope or a solid-phase cytometer.

In another aspect, the present invention also relates to the use of the method of the invention in determining whether a sample contains at least one microorganism of interest, in determining whether a sample is sterile, or in determining the bioburden of a sample.

In another aspect, the present invention also relates to the use of a kit to detect and/or enumerate a microorganism or a group of microorganisms of interest according to the method of the invention, said kit comprising
(i)—at least one fluorescent dye, preferably as defined above,
and optionally
   at least one membrane filter, preferably as defined above, and/or
   at least one container comprising a solid growth medium containing nutrients to support growth of the microorganism or a group of microorganisms of interest, or materials needed to obtained said at least one container and/or
   a leaflet providing instructions to use said kit, or
(ii)—at least one container comprising a solid growth medium containing at least one fluorescent dye, preferably as defined above, and nutrients to support growth of the microorganism or a group of microorganisms of interest, or materials needed to obtained said at least one container, and
and optionally
   at least one membrane filter, preferably as defined above, and/or
   a leaflet providing instructions to use said kit.

### BRIEF DESCRIPTION OF THE FIGURES

FIG. 1: A. acidoterrestris 24 h, PVDF membrane, MB660R-DBCO 100 μM, Light power 100%, Gain 1, Exposure time 100 ms.

FIG. 2: E. coli 17 h, MCE black membrane, Alexa Fluor™ 350-NHS ester 500 μM, Light power 50%, Gain 1, Exposure time 200 ms.

FIG. 3: E. coli 17 h, PVDF membrane, Alexa Fluor™ 350-NHS ester 150 μM, Light power 20%, Gain 1, Exposure time 200 ms.

FIG. 4: E. coli 17 h, MCE black membrane, Alexa Fluor™ 405-DBCO 500 μM, Light power 50%, Gain 1, Exposure time 200 ms.

FIG. 5: E. coli 15 h, PVDF membrane, MB660R-DBCO 250 μM, Light power 100%, Gain 1, Exposure time 100 ms.

FIG. 6: E. coli 18 h, MCE black membrane, Sulfo-Cy5-acid 150 μM, Light power 100%, Gain 1, Exposure time 400 ms.

FIG. 7: E. coli 18 h, PVDF membrane, Sulfo-Cy5-acid 50 μM, Light power 100%, Gain 1, Exposure time 400 ms.

FIG. 8: C. albicans 21 h, PVDF membrane, MB660R-DBCO 250 μM, Light power 100%, Gain 1, Exposure time 100 ms.

FIG. 9: A. brasiliensis 21 h, PVDF membrane, MB660R-DBCO 250 μM, Light power 100%, Gain 1, Exposure time 200 ms.

FIG. 10: Mix C. albicans+A. brasiliensis 20 h, PVDF membrane, Sulfo-Cy5-acid 50 μM, Light power 100%, Gain 1, Exposure time 400 ms.

FIG. 11: Mix C. albicans+A. brasiliensis 21 h, MCE black membrane, Alexa Fluor™ 405-DBCO500 μM, Light power 50%, Gain 1, Exposure time 200 ms.

FIG. 12: Mix C. albicans+A. brasiliensis 21 h, MCE black membrane, Alexa Fluor™ 350-NHS ester 500 μM, Light power 50%, Gain 1, Exposure time 200 ms.

FIG. 13: Mix C. albicans+A. brasiliensis 21 h, PVDF membrane, Alexa Fluor™ 350-NHS ester 500 μM, Light power 20%, Gain 1, Exposure time 300 ms.

FIG. 14: E. faecalis+C. albicans+A. brasiliensis 24 h, TSA agar plate 25° C., PVDF membrane, MB660R 100 μM+Sulfo-Cy5-acid 100 μM, Light power 100%, Gain 1, Exposure time 200 ms.

FIG. 15: Structures of fluorescent dyes.

FIG. 16: B. subtilis, black or white membrane, MB660R-DBCO (50 μM) and Sulfo-Cy5-acid (50 μM), using different methods to contact the fluorescent dyes with the bacteria.

FIG. 17: Alicyclobacillus acidoterrestris, Alicyclobacillus cycloheptanicus and Alicyclobacillus herbarius, MB660R-DBCO, detection on a membrane placed on the agar medium or directly on the agar medium (without membrane).

### DETAILED DESCRIPTION OF THE INVENTION

The inventors herein demonstrated that microorganisms may be cultured on a solid medium in the presence of fluorescent dyes in order to allow early detection of microcolonies. Indeed, they surprisingly found that fluorescent dyes do not exhibit significant toxicity and thus do not affect the growth of microorganisms to be detected. The presence of these dyes during the growth phase of microorganisms allows very early detection and counting of the microorganisms at the microcolony stage. In particular, the inventors showed that this method may be used to rapidly detect and enumerate thermophilic acidophile bacteria (TAB), acetic acid bacteria (AAB), lactic acid bacteria (LAB), anaerobic bacteria, aerobic mesophilic bacteria, yeasts, molds and bacteria of relevant importance in UTI diagnosis, but also to assess sterility or bioburden of a sample. The method developed by the inventors provides faster and more precise results than other methods while being a cost effective method using standard equipment and reagents, having an easy workflow which does not required highly skilled specialists to be put into practice, and being able to distinguish live and dead microorganisms. This method thus fulfils the need for a rapid, sensitive, user friendly and cost effective method for detecting and enumerating microorganisms in a sample of interest.

Accordingly, in a first aspect, the present invention relates to a method for detecting or enumerating a microorganism or a group of microorganisms of interest comprising
a) contacting, in a container, a sample suspected of containing said microorganism or group of microorganisms with a solid growth medium containing nutrients to support growth of said microorganism or group of microorganisms and with at least one fluorescent dye;
b) incubating said container under conditions and for a time sufficient to form microcolonies of said microorganism or group of microorganisms; and
c) detecting or enumerating microcolonies emitting a fluorescent signal of said at least one fluorescent dye,
thereby detecting or enumerating said microorganism or group of microorganisms contained in the sample.

The method of the invention can be used to detect a microorganism or a group of microorganisms in a sample, i.e. to reveal the presence of microorganisms present in the sample through the detection of microcolonies in step c) of the method. It can also be used to enumerate a microorganism or a group of microorganisms in a sample. In this case, it is assumed that each microcolony detected in step c) arises from an individual cell that has undergone cell division. Therefore, by counting the number of microcolonies, and optionally accounting for the dilution or concentration factor, the number of said microorganisms in the sample can be determined. The method of the invention can also be used to identify a microorganism or a group of microorganisms. As used herein, the term “identification” does not necessarily require the determination of genus and species of a given microcolony. This term may refer to a classification of a microorganism in a taxonomic group (at any rank) or in a specific group (e.g. thermophilic acidophilic bacteria, acetic acid bacteria, lactic acid bacteria, yeasts or molds, aerobic mesophilic bacteria, anaerobic bacteria).

Step a) of the method of the invention comprises contacting, in a container, a sample suspected of containing a microorganism or a group of microorganisms with a solid growth medium containing nutrients to support growth of said microorganism or group of microorganisms and with at least one fluorescent dye.

Step a) may comprise
a1) contacting a sample suspected of containing said microorganism or group of microorganisms with at least one fluorescent dye, and
a2) contacting, in a container, said sample with a solid growth medium containing nutrients to support growth of said microorganism or group of microorganisms,
steps a1) and a2) being carried out simultaneously or sequentially, in any order.

In some embodiments, step a1) is carried out before step a2). Optionally, before step a2), the microorganisms of the sample are concentrated on a membrane filter. In particular, the sample may be concentrated on a membrane filter before or after step a1), preferably after step a1). In particular, said at least one fluorescent dye may be filtered on the same membrane than the sample, before or after the filtration of the sample.

In some particular embodiments, steps a1) and a2) are carried out simultaneously.

The method may further comprise, before step a), providing a sample suspected of containing the microorganism or group of microorganisms of interest.

### Samples

The sample may be obtained from a liquid (e.g. water, fruit juices, beer, wine, biological fluids such as urine), a solid (e.g. food, pharmaceutical or cosmetic products, medical devices or any solid surfaces) or a gas (e.g. air). Depending on the form of the product/environment to be tested, the sample may be directly used in the method of the invention or may be submitted to preliminary step(s).

In particular, the sample can be a liquid sample or a liquified sample. As used herein, the term “liquified sample” refers to a liquid sample obtained from a solid sample. In some cases, the solid sample may be dissolved or suspended in a liquid medium through physical and/or chemical treatments. Microorganisms may also be extracted from different surfaces or devices using any method known by the skilled person such as swabbing methods, friction methods, e.g. using wipes, printing methods, e.g. by agar contact method, rinsing or immersion methods, or sonication methods, in particular to dislodge biofilms (see e.g. Ismail et al., Int J Environ Res Public Health. 2013 Nov. 14; 10(11):6169-83, incorporated herein by reference).

Liquid samples may contain suspended solids. However, if necessary, remaining suspended solids may be eliminated from the liquid medium using a suitable method, preferably using a method minimizing the loss of microorganisms, e.g. by low speed centrifugation or filtration using suitable pore size. The liquid medium used to suspend solid sample may be any suitable solvent such as sterile water, buffer solution or liquid culture medium.

Optionally, the liquid or liquified sample may be diluted (e.g. serial dilutions) or concentrated prior step a) using any suitable method such as centrifugation or filtration.

The sample used/analyzed in the method of the invention can be any sample for which it is sought to determine whether it is contaminated with microorganisms.

Examples of samples include, but are not limited to, biological samples (e.g. saliva, nasopharyngeal, urine, fecal, blood, plasma, cerebrospinal fluid or mucus sample), environmental sample (e.g. residential, commercial or industrial water, waste water, cooling water, boiler water, ground water, recreational water, process water, effluent of water treatment unit, soil, or other environmental material), medical devices or any part thereof, foods or beverages for human or animal consumption (e.g. dairy products, raw materials, drinking water, fruit juices, beers, wines, water used in the composition of the product), pharmaceutical or cosmetic products, as well as ingredients of such foods, beverages, pharmaceutical or cosmetic products.

### Membrane Filter

In some preferred embodiments, the method may further comprise, before step a), concentrating the microorganisms of the sample on a membrane filter.

In step a), this membrane is then contacted with the solid growth medium and at least one fluorescent dye as described below. Alternatively, the sample may be contacted with said at least one fluorescent dye before the filtration, or after the filtration but before the contact with the solid growth medium. In particular, the sample may be mixed with said at least one fluorescent dye and then may be filtered on the membrane or said at least one fluorescent dye may be filtered on the same membrane than the sample, before or after the filtration of the sample. In particular, the fluorescent dye(s) may be added to the sample prior to filtration/concentration on the membrane or may be filtered on the same membrane prior or after the filtration of the sample, preferably after the filtration of the sample. The membrane filter may be then placed on the surface of said growth medium.

The passage of nutrients through the filter during incubation allows the growth of organisms in the form of microcolonies, on the upper surface of the membrane.

The liquid, liquified or gaseous sample may be filtered/concentrated using a sterile membrane filter suitable to retain microorganisms contained in the sample, typically a microfiltration membrane filter having pore sizes smaller than the target microorganisms. The sample may be passed through the membrane using a filter funnel and vacuum system. Preferably, the membrane filter has a nominal pore size not greater than 0.45 μm, a pore size of 0.22 μm to 0.45 μm. The diameter of the filter may depend on the device used to filtrate the sample and the size of the container containing the growth medium. For example, the container may be a 90 mm or 55 mm diameter Petri dish and the membrane may have a diameter of 47 mm.

The membrane filter may be made of any suitable material such as mixed cellulose esters (MCE), polyvinylidene fluoride (PVDF), polyester sulfone (PES), nitrocellulose, polytetrafluoroethylene, polycarbonate or nylon. Preferably, the membrane filter is made of mixed cellulose esters (MCE), polyvinylidene fluoride (PVDF), nitrocellulose, polytetrafluoroethylene, polycarbonate or nylon. More preferably the membrane filter is made of mixed cellulose esters (MCE) or polyvinylidene fluoride (PVDF). The membrane filter may be white or colored, e.g. black, and/or may be grid membrane.

The volume of sample to be filtered may depend on the nature of the sample and the expected microorganism content. This volume is typically between 1 mL to 1 L and can be easily adjusted by the skilled person.

### Microorganisms

The method of the invention allows detection or enumeration of a variety of microorganisms or groups of microorganisms that can be grown in a culture device, and in particular on a suitable solid growth medium, i.e. culturable microorganisms.

As used herein, the term “microorganism” refers to a bacterium, an archaebacterium or a microscopic fungus. Preferably, this term refers to a bacterium, a yeast (i.e. unicellular microscopic fungus) or a mold (i.e. multicellular and filamentous microscopic fungus). In particular, the method of the invention allows detection or enumeration of living microorganisms. As used herein, the term “living microorganisms” refers to microorganisms capable of multiplying when incubated in a suitable liquid culture medium or on a suitable solid culture medium. Indeed, contrary to some other methods of the prior art, the method of the invention does not detect dead microorganisms or fragments thereof such as DNA or cell fragments, and can this clearly distinguishes living microorganisms.

As used herein, the term “group of microorganisms” refers to a group comprising at least two distinct microorganisms. Said microorganisms may be selected from the group consisting of bacteria, archaebacteria and microscopic fungi, and combinations thereof, preferably selected from the group consisting of bacteria, yeasts and molds, and combinations thereof. In particular, the group of microorganisms may comprise two or more bacteria, two or more fungi, or a mix of one or more bacteria and one or more fungi.

The microorganisms of interest may be pathogenic or non-pathogenic microorganisms. Preferably, the microorganisms are pathogenic microorganisms or non-pathogenic microorganisms which can deteriorate or spoil a product (e.g. food, beverage, medical device, pharmaceutical or cosmetic product) or an environment (e.g. swimming-pool, groundwater).

In some embodiments, the microorganism or group of microorganisms of interest are bacteria. Bacteria may be an aerobic, anaerobic or facultative anaerobic bacteria, and Gram negative or Gram positive bacteria.

In particular, the microorganism or group of microorganisms of interest may belong to the group of thermophilic acidophilic bacteria (TAB), the group of acetic acid bacteria (AAB), the group of lactic acid bacteria (LAB), the group of anaerobic bacteria, the group of aerobic mesophilic bacteria, the group of Gram negative bacteria, the group of Gram positive bacteria, the group of heterotrophic bacteria, or a combination thereof. More particularly, the microorganism or group of microorganisms of interest may belong to the group of thermophilic acidophilic bacteria (TAB), the group of acetic acid bacteria (AAB), the group of lactic acid bacteria (LAB), the group of anaerobic bacteria, the group of aerobic mesophilic bacteria, the group of Gram negative bacteria or the group of Gram positive bacteria, or a combination thereof.

In an embodiment, the microorganism or group of microorganisms of interest belongs to the group of Alicyclobacillus spp., also known as thermophilic acidophilic bacteria (TAB).

Alicyclobacillus spp. are spore-forming bacteria that can grow in acid conditions at raised temperatures (up to 70° C.). The spores are able to survive typical pasteurization procedures, and as a result these bacteria are potential spoilage organisms of fruit juices, juice related products (fruit juices from concentrates, concentrated fruit juices, water extracted fruit juices, dehydrated/powdered fruit juices, fruit nectars and non-alcoholic or alcoholic beverages with fruit products like flavored waters) or syrups. Typically, TAB are not harmful but spoilage occurs if the strain produces guaiacol, a natural phenolic chemical with a smoky taste and odor, which taints the fruit juice, or other off-flavor substances. The most common guaiacol producing TAB is Alicyclobacillus acidoterrestris.

Preferably, the microorganism or group of microorganisms of interest belonging to the group of Alicyclobacillus spp. is selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius and Alicyclobacillus contaminans, and combinations thereof.

In another embodiment, the microorganism or group of microorganisms of interest belongs to the group of acetic acid bacteria (AAB).

AAB are strictly aerobic microorganisms, catalase-positive and oxidase-negative, ellipsoidal to rod-shaped cells that can occur singly, in pairs or chains. They are also mesophilic microorganisms, and their optimum growth temperature is between 25 and 30° C. The optimum pH for their growth is 5.0-6.5, but they can also grow at lower pH values.

These bacteria are widespread in nature and play an important role in the production of food and beverages, such as vinegar. However, the unwanted growth of AAB in other fermented beverages, such as wine, cider, beer and other soft drinks causes an undesirable sour taste. As illustration, Gluconobacter strains, belonging to this group, are considered to be typical spoilers of soft drinks. Indeed, on these substrates, the sugars and alcohols are incompletely oxidized, leading to the accumulation of organic acids, such as the production of acetic acid from ethanol or gluconic acid from glucose.

Preferably, the microorganism or group of microorganisms of interest belonging to the group of acetic acid bacteria (AAB) is selected from bacteria belonging to the genera Acetobacter, Acidomonas, Asaia, Gluconacetobacter, Gluconobacter, Ameyamaea, Bombella, Commensalibacter, Endobacter, Granulibacter, Komagataeibacter, Kozakia, Neoasaia, Neokomagataea, Nguyenibacter, Saccharibacter, Swaminathania, Swingsia and Tanticharoenia, and combinations thereof.

More preferably, the microorganism or group of microorganisms of interest belonging to the group of acetic acid bacteria (AAB) is selected from bacteria belonging to the genera Acetobacter such as Acetobacter pasteurianus, Acetobacter malorum, Acetobacter cerevisiae, Acetobacter oeni, Acetobacter pomorum and Acetobacter aceti, Asaia such as Asaia bogorensis, Asaia siamensis, Asaia krungthepensis and Asaia lannaensis, Gluconacetobacter such as Gluconoacetobacter liquefaciens, Gluconacetobacter entanii and Gluconacetobacter intermedius. Gluconobacter such as Gluconobacter oxydans, and combinations thereof. Even more preferably, the microorganism or group of microorganisms of interest belonging to the group of acetic acid bacteria (AAB) is selected from the group consisting of Acetobacter aceti, Gluconoacetobacter liquefaciens, Gluconobacter oxydans and Asaia siamensis, and combinations thereof.

In another embodiment, the microorganism or group of microorganisms of interest belongs to the group of lactic acid bacteria (LAB).

This group of bacteria often play a positive role in the food industry but they can also cause major spoilage in particular in wine and foods having low pH or being vacuum-packed. Furthermore, these bacteria may be considered as indicators since conditions favorable to their growth also favor Clostridium botulinum, a highly pathogenic bacterium. These bacteria form a heterogeneous group, but have in common that they are catalase negative (with some strains able to produce a heme-dependent catalase), non-spore-forming, strictly fermentative, facultative aerobic, and producing lactic acid as a major product of glucose fermentation.

The microorganism or group of microorganisms of interest belonging to the group of lactic acid bacteria (LAB) is preferably selected from bacteria belonging to the genera Lactobacillus (e.g. Lactobacillus casei, Lactobacillus plantarum, Lactobacillus sakei, Lactobacillus brevis, Lactobacillus lindneri, Lactobacillus fructivorans, Lactobacillus acidophilus), Leuconostoc (e.g. Leuconostoc gelidum, Leuconostoc gasicomitatum and Leuconostoc mesenteroides), Pediococcus (e.g. Pediococcus damnosus, Pediococcus acidilactici, Pediococcus pentosaceus, Pediococcus parvulus, Pediococcus inopinatus, Pediococcus halophilus, Pediococcus dextrinicus, and Pediococcus urinaeequi), Lactococcus (e.g. Lactococcus piscium), Enterococcus (e.g. Enterococcus faecalis and Enterococcus faecium), Carnobacterium (e.g. Carnobacterium divergens, Carnobacterium maltaromaticum), Streptococcus (e.g. Streptococcus lactis) and Weissella (e.g. Weissella confusa, Weissella viridescens), and combinations thereof.

More preferably, the microorganism or group of microorganisms of interest belonging to the group of lactic acid bacteria (LAB) is selected from bacteria belonging to the genera Lactobacillus and Weissella, and combinations thereof.

Even more preferably, the microorganism or group of microorganisms of interest belonging to the group of lactic acid bacteria (LAB) is selected from the group consisting of Lactobacillus casei, Lactobacillus plantarum, Weissella confusa, and combinations thereof.

In another particular embodiment, the microorganism or group of microorganisms of interest belongs to the group of anaerobic bacteria.

Anaerobic bacteria may be classified into three categories: obligate anaerobes, which are harmed by the presence of oxygen, aerotolerant organisms, which cannot use oxygen for growth, but tolerate its presence and facultative anaerobes, which can grow without oxygen but use oxygen if it is present. Most pathogenic foodborne microorganisms are facultative anaerobes. Anaerobic bacteria, in particular Clostridium bacteria, may spoil vacuum packaged foods or canned foods. Anaerobic bacteria, in particular Cutibacterium acnes, may be also involved in postoperative infections due to their ability to persist on body implants and surgical devices.

The microorganism or group of microorganisms of interest belonging to the group of anaerobic bacteria is preferably selected from bacteria belonging to the Clostridium genus such as Clostridium sporogenes, Clostridium algidicarnis, Clostridium frigoris, Clostridium bowmanii, Clostridium frigidicarmis, Clostridium ruminantium, Clostridium estertheticum, Clostridium gasigenes, Clostridium perfringens or Clostridium botulinum, Cutibacterium genus such as Cutibacterium acnes, and Brochothrix genus such as Brochothrix thermosphacta and combinations thereof.

More preferably, the microorganism or group of microorganisms of interest belonging to the group of anaerobic bacteria is selected from bacteria belonging to the Clostridium genus or Cutibacterium genus, and combinations thereof, even more preferably, from the group consisting of Clostridium sporogenes and Cutibacterium acnes, and combinations thereof.

In another embodiment, the microorganism or group of microorganisms of interest belongs to the group of aerobic mesophilic bacteria (AMB). Aerobic mesophilic bacteria are bacteria growing aerobically at mesophilic temperatures, i.e. between 25° C. and 40° C. Detection and enumeration of aerobic mesophilic bacteria are a key indicator for quality control of food products, cosmetic products, medical devices and non-sterile pharmaceutical products.

The microorganism or group of microorganisms of interest belonging to the group of aerobic mesophilic bacteria is preferably selected from bacteria belonging to the genera Methylobacterium (e.g. Methylobacterium extorquens), Pseudomonas (e.g. Pseudomonas aeruginosa), Bacillus (e.g. Bacillus subtilis, Bacillus cereus), Escherichia (e.g. Escherichia coli), Staphylococcus (e.g. Staphylococcus aureus), Acinetobacter (e.g. Acinetobacter baumannii), Cronobacter (e.g. Cronobacter sakazakii), Klebsiella (e.g. Klebsiella pneumoniae), Salmonella (e.g. Salmonella typhimurium), Enterococcus (e.g. Enterococcus faecalis), Listeria (e.g. Listeria grayi, Listeria monocytogenes), Shigella (e.g. Shigella sonnei), Vibrio (e.g. Vibrio parahaemolyticus, Vibrio cholerae, Vibrio vulnificus), Campylobacter (e.g. Campylobacter jejuni, Campylobacter coli), Yersinia (e.g. Yersinia enterocolitica), Kocuria (e.g. Kocuria rhizophila) and Burkholderia (e.g. Burkholderia cepacia), and combinations thereof.

More preferably, the microorganism or group of microorganisms of interest belonging to the group of aerobic mesophilic bacteria is selected from bacteria belonging to the genera Methylobacterium, Pseudomonas, Bacillus, Escherichia, Staphylococcus, Acinetobacter, Cronobacter, Klebsiella, Salmonella, Enterococcus, Listeria, Shigella, Kocuria, Burkholderia, and combinations thereof. The microorganism or group of microorganisms of interest belonging to the group of aerobic mesophilic bacteria may be more preferably selected from bacteria belonging to the genera Methylobacterium, Pseudomonas, Bacillus, Escherichia, Staphylococcus, Acinetobacter, Cronobacter, Klebsiella, Salmonella, Enterococcus, Shigella, Kocuria, Burkholderia, and combinations thereof.

Even more preferably, the microorganism or group of microorganisms of interest belonging to the group of aerobic mesophilic bacteria is selected from the group consisting of Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Listeria grayi, Shigella sonnei, Kocuria rhizophila and Burkholderia cepacia, and combinations thereof. The microorganism or group of microorganisms of interest belonging to the group of aerobic mesophilic bacteria is even more preferably selected from the group consisting of Methylobacterium extorquens, Pseudomonas aeruginosa, Bacillus subtilis, Escherichia coli, Staphylococcus aureus, Acinetobacter baumannii. Cronobacter sakazakii, Klebsiella pneumoniae, Salmonella typhimurium, Enterococcus faecalis, Shigella sonnei, Kocuria rhizophila and Burkholderia cepacia, and combinations thereof.

In another embodiment, the microorganism or group of microorganisms of interest belongs to the group of Gram negative bacteria or Gram positive bacteria.

Gram negative bacteria are characterized by their cell envelopes, which are composed of a thin peptidoglycan cell wall sandwiched between an inner cytoplasmic cell membrane and a bacterial outer membrane. They are an important medical challenge, as their outer membrane protects them from many antibiotics (including penicillin); detergents that would normally damage the peptidoglycans of the (inner) cell membrane; and lysozyme, an antimicrobial enzyme produced by animals that forms part of the innate immune system. Additionally, the outer leaflet of this membrane comprises a complex lipopolysaccharide (LPS) whose lipid A component can cause a toxic reaction when these bacteria are lysed by immune cells. They are, for example, implicated in most cases of urinary tract infection. Escherichia coli is the most common gram-negative pathogen however other gram-negative bacteria have a significant importance such as Klebsiella, Chlamydia, Proteus, Pseudomonas, Enterobacter, Acinetobacter, Serratia, Haemophilus, Yersinia and Salmonella.

The microorganism or group of microorganisms of interest belonging to the group of Gram-negative bacteria is preferably selected from bacteria belonging to the genera Escherichia (e.g. Escherichia coli), Klebsiella (e.g. Klebsiella pneumoniae), Proteus (e.g. Proteus mirabilis), Chlamydia (e.g. Chlamydia trachomatis), Pseudomonas (e.g. Pseudomonas aeruginosa), Enterobacter (e.g. Enterobacter faecalis and Enterobacter aerogenes), Acinetobacter (e.g. Acinetobacter baumannii), Cronobacter (e.g. Cronobacter sakazakii), Shigella (e.g. Shigella sonnei), Kocuria (e.g. Kocuria rhizophila), Burkholderia (e.g. Burkholderia cepacia), Serratia (e.g. Serratia marcescens), Haemophilus (e.g. Haemophilus influenzae) and Salmonella (e.g. Salmonella typhimurium), and combinations thereof. More preferably, the microorganism or group of microorganisms of interest belonging to the group of Gram-negative bacteria is selected from bacteria belonging to the genera Escherichia (e.g. Escherichia coli), Klebsiella (e.g. Klebsiella pneumoniae), Proteus (e.g. Proteus mirabilis), Pseudomonas (e.g. Pseudomonas aeruginosa), Enterobacter (e.g. Enterobacter faecalis and Enterobacter aerogenes), Acinetobacter (e.g. Acinetobacter baumannii), Cronobacter (e.g. Cronobacter sakazakii), Shigella (e.g. Shigella sonnei), Kocuria (e.g. Kocuria rhizophila), Burkholderia (e.g. Burkholderia cepacia), Serratia (e.g. Serratia marcescens), and Salmonella (e.g. Salmonella typhimurium), and combinations thereof, in particular is selected from Escherichia coli, Klebsiella pneumoniae, Proteus mirabilis, Pseudomonas aeruginosa, Enterobacter aerogenes, Acinetobacter baumannii, Cronobacter sakazakii, Shigella sonnei, Kocuria rhizophila, Burkholderia cepacia, Serratia marcescens, and Salmonella typhimurium.

More particularly, the microorganism or group of microorganisms of interest belonging to the group of Gram-negative bacteria may be selected from Escherichia, Proteus and Pseudomonas bacteria, and combination thereof, more preferably from Escherichia coli, Proteus mirabilis, Pseudomonas aeruginosa and combination thereof. Even more particularly, the microorganism or group of microorganisms of interest belonging to the group of Gram-negative bacteria may be selected from Escherichia and Pseudomonas bacteria, and combination thereof, more preferably from Escherichia coli, Pseudomonas aeruginosa and combination thereof, and even more preferably from Escherichia coli bacteria.

In particular, the microorganism or group of microorganisms of interest belonging to the group of Gram-negative bacteria may belong to the Enterobacteriaceae family including for example Escherichia, Klebsiella, Enterobacter and Salmonella, and combinations thereof, more particularly Escherichia coli, Klebsiella pneumoniae, Enterobacter aerogenes and Salmonella typhimurium, and combinations thereof.

Gram positive bacteria are also clinically relevant. In particular, among gram-positive bacteria isolated in bacterial UTIs, Staphylococcus saprophyticus, Staphylococcus aureus, Enterococcus faecalis and Streptococcus agalactiae are the most common Gram positive pathogens.

The microorganism or group of microorganisms of interest belonging to the group of Gram-positive bacteria is preferably selected from bacteria belonging to the genera Staphylococcus, Enterococcus, Streptococcus, and combinations thereof. More particularly, the microorganism or group of microorganisms of interest belonging to the group of Gram-positive bacteria may be selected from Staphylococcus aureus and Enterococcus faecalis, and combination thereof.

In another embodiment, the microorganism or group of microorganisms of interest belongs to the group of heterotrophic bacteria, in particular heterotrophic bacteria which are typically recoverable from pharmaceutical water systems, i.e. mains (or potable) water, purified water and WFI (Water-for-Injection) (see e.g. Tim Sandle, September 2015. SOJ Microbiology & Infectious Diseases 3(2):1-8, herein incorporated by reference). Indeed, water is a key part of the pharmaceutical industry. Water is used for cleaning; as an ingredient for aqueous sterile and non-sterile products; for hand washing; and as the steam supply to autoclaves, among other uses. Due to its criticality in pharmaceutical production, microbiological control of water is of great importance. Because water is ever present, each grade of pharmaceutical water is a potential source of microbiological contamination, especially when not properly controlled. In pharmaceutical 3 types of water are present: a) Mains (or potable) Water; b) Purified Water; c) WFI (Water-for-Injection) Each of these is of a different grade and the list descends with an increased expectation of microbial control (that is tighter limits apply to Water-for-Injection that for mains water). Mains water is supplied by a utility company and is of “drinking water” (potable water) quality. The results from the monitoring of water systems are assessed from heterotrophic microbial counts against pre-defined alert and action levels.

The microorganism or group of microorganisms of interest belonging to the group of heterotrophic bacteria is preferably selected from bacteria belonging to the genera Acinetobacter (e.g Acinetobacter baumanii), Aeromonas (e.g. Aeromonas hydrophila), Brevundimonas (e.g. Brevundimonas diminuta), Burkholderia (e.g. Burkholderia cepacia), Citrobacter (e.g. Citrobacter freundii), Edwardsiella (e.g. Edwardsiella tarda), Enterobacter (e.g. Enterobacter aerogenes), Escherichia (e.g. Escherichia coli), Ochrobactrum (e.g. Ochrobactrum anthropic), Klebsiella (e.g. Klebsiella pneumoniae), Methylobacterium (e.g. Methylobacterium extorquens), Moraxella (e.g. Moraxella osloensis), Pantoea (e.g. Pantoea agglomerans), Proteus (e.g. Proteus mirabilis), Pseudomonas (e.g. Pseudomonas aeruginosa, Pseudomonas fluorescens), Ralstonia (e.g. Ralstonia pickettii), Salmonella (e.g. Salmonella typhimurium), Serratia (e.g. Serratia marcescens), Shigella (e.g. Shigella sonnei), Sphingomonas (e.g. Sphingomonas paucimobilis), Stenotrophomonas (e.g. Stenotrophomonas maltophilia), Vibrio (e.g Vibrio parahaemolyticus), Yersinia (e.g. Yersinia enterocolitica), Bacillus (e.g. Bacillus subtilis), Enterococcus (e.g. Enterococcus faecalis), Micrococcus (e.g. Micrococcus luteus (Kocuria rhizophila)), Staphylococcus (e.g. Staphylococcus aureus), and combinations thereof. More preferably, the microorganism or group of microorganisms of interest belonging to the group of heterotrophic bacteria is selected from bacteria belonging to the genera Acinetobacter (e.g Acinetobacter baumanii), Aeromonas (e.g. Aeromonas hydrophila), Brevundimonas (e.g. Brevundimonas diminuta), Burkholderia (e.g. Burkholderia cepacia), Citrobacter (e.g. Citrobacter freundii), Edwardsiella (e.g. Edwardsiella tarda), Enterobacter (e.g. Enterobacter aerogenes), Escherichia (e.g. Escherichia coli), Ochrobactrum (e.g. Ochrobactrum anthropic), Klebsiella (e.g. Klebsiella pneumoniae), Methylobacterium (e.g. Methylobacterium extorquens), Moraxella (e.g. Moraxella osloensis), Pantoea (e.g. Pantoea agglomerans), Proteus (e.g. Proteus mirabilis), Pseudomonas (e.g. Pseudomonas aeruginosa, Pseudomonas fluorescens), Ralstonia (e.g. Ralstonia pickettii), Salmonella (e.g. Salmonella typhimurium), Serratia (e.g. Serratia marcescens), Shigella (e.g. Shigella sonnei), Sphingomonas (e.g. Sphingomonas paucimobilis), Stenotrophomonas (e.g. Stenotrophomonas maltophilia), Yersinia (e.g. Yersinia enterocolitica), Bacillus (e.g. Bacillus subtilis), Enterococcus (e.g. Enterococcus faecalis), Micrococcus (e.g. Micrococcus luteus (Kocuria rhizophila)), Staphylococcus (e.g. Staphylococcus aureus), and combinations thereof.

More particularly, the microorganism or group of microorganisms of interest belonging to the group of heterotrophic bacteria is preferably selected from Acinetobacter baumanii, Aeromonas hydrophila, Brevundimonas diminuta, Burkholderia cepacia, Citrobacter freundii, Edwardsiella tarda, Enterobacter aerogenes, Escherichia coli, Ochrobactrum anthropic, Klebsiella pneumoniae, Methylobacterium extorquens, Moraxella osloensis, Pantoea agglomerans, Proteus mirabilis, Pseudomonas aeruginosa, Pseudomonas fluorescens, Ralstonia pickettii, Salmonella typhimurium, Serratia marcescens, Shigella sonnei, Sphingomonas paucimobilis, Stenotrophomonas maltophilia, Vibrio parahaemolyticus, Yersinia enterocolitica, Bacillus subtilis, Enterococcus faecalis, Micrococcus luteus (Kocuria rhizophila), Staphylococcus aureus, and combinations thereof. Even more particularly, the microorganism or group of microorganisms of interest belonging to the group of heterotrophic bacteria may be selected from Acinetobacter baumanii, Aeromonas hydrophila, Brevundimonas diminuta, Burkholderia cepacia, Citrobacter freundii, Edwardsiella tarda, Enterobacter aerogenes, Escherichia coli, Ochrobactrum anthropic, Klebsiella pneumoniae, Methylobacterium extorquens, Moraxella osloensis, Pantoea agglomerans, Proteus mirabilis, Pseudomonas aeruginosa, Pseudomonas fluorescens, Ralstonia pickettii, Salmonella typhimurium, Serratia marcescens, Shigella sonnei, Sphingomonas paucimobilis, Stenotrophomonas maltophilia, Yersinia enterocolitica, Bacillus subtilis, Enterococcus faecalis, Micrococcus luteus (Kocuria rhizophila), Staphylococcus aureus, and combinations thereof.

In particular, the microorganism or group of microorganisms of interest may be selected from the group consisting of bacteria belonging to the genera Alicyclobacillus (e.g. Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans), Acetobacter (e.g. Acetobacter aceti), Gluconoacetobacter (e.g. Gluconoacetobacter liquefaciens), Gluconobacter (e.g. Gluconobacter oxydans), Asaia (e.g. Asaia siamensis), Lactobacillus (e.g. Lactobacillus casei, Lactobacillus plantarum), Weissella (e.g. Weissella confusa), Clostridium (e.g. Clostridium sporogenes), Cutibacterium (e.g Cutibacterium acnes), Methylobacterium (e.g. Methylobacterium extorquens), Pseudomonas (e.g. Pseudomonas aeruginosa), Bacillus (e.g. Bacillus subtilis), Escherichia (e.g. Escherichia coli), Staphylococcus (e.g. Staphylococcus aureus), Acinetobacter (e.g. Acinetobacter baumannii), Cronobacter (e.g. Cronobacter sakazakii), Klebsiella (e.g. Klebsiella pneumoniae), Salmonella (e.g. Salmonella typhimurium), Enterococcus (e.g. Enterococcus faecalis), Listeria (e.g. Listeria grayi), Shigella (e.g. Shigella sonnei), Micrococcus (also named Kocuria) (e.g. Micrococcus luteus (Kocuria rhizophila)), Burkholderia (e.g. Burkholderia cepacia), Aeromonas (e.g. Aeromonas hydrophila), Brevundimonas (e.g. Brevundimonas diminuta), Citrobacter (e.g. Citrobacter freundii), Edwardsiella (e.g. Edwardsiella tarda), Enterobacter (e.g. Enterobacter aerogenes), Ochrobactrum (e.g. Ochrobactrum anthropic), Moraxella (e.g. Moraxella osloensis), Pantoea (e.g. Pantoea agglomerans), Proteus (e.g. Proteus mirabilis), Ralstonia (e.g. Ralstonia pickettii), Serratia (e.g. Serratia marcescens), Sphingomonas (e.g. Sphingomonas paucimobilis), Stenotrophomonas (e.g. Stenotrophomonas maltophilia), Vibrio (e.g Vibrio parahaemolyticus), Yersinia (e.g. Yersinia enterocolitica), and combinations thereof. More particularly, the microorganism or group of microorganisms of interest may be selected from the group consisting of bacteria belonging to the genera Alicyclobacillus (e.g. Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans), Acetobacter (e.g. Acetobacter aceti), Gluconoacetobacter (e.g. Gluconoacetobacter liquefaciens), Gluconobacter (e.g. Gluconobacter oxydans), Asaia (e.g. Asaia siamensis), Lactobacillus (e.g. Lactobacillus casei. Lactobacillus plantarum), Weissella (e.g. Weissella confusa), Clostridium (e.g. Clostridium sporogenes), Cutibacterium (e.g Cutibacterium acnes), Methylobacterium (e.g. Methylobacterium extorquens), Pseudomonas (e.g. Pseudomonas aeruginosa), Bacillus (e.g. Bacillus subtilis), Escherichia (e.g. Escherichia coli), Staphylococcus (e.g. Staphylococcus aureus), Acinetobacter (e.g. Acinetobacter baumannii), Cronobacter (e.g. Cronobacter sakazakii), Klebsiella (e.g. Klebsiella pneumoniae), Salmonella (e.g. Salmonella typhimurium), Enterococcus (e.g. Enterococcus faecalis), Shigella (e.g. Shigella sonnei), Micrococcus (also named Kocuria) (e.g. Micrococcus luteus (Kocuria rhizophila)), Burkholderia (e.g. Burkholderia cepacia), Aeromonas (e.g. Aeromonas hydrophila), Brevundimonas (e.g. Brevundimonas diminuta), Citrobacter (e.g. Citrobacter freundii), Edwardsiella (e.g. Edwardsiella tarda), Enterobacter (e.g. Enterobacter aerogenes), Ochrobactrum (e.g. Ochrobactrum anthropic), Moraxella (e.g. Moraxella osloensis), Pantoea (e.g. Pantoea agglomerans), Proteus (e.g. Proteus mirabilis), Ralstonia (e.g. Ralstonia pickettii), Serratia (e.g. Serratia marcescens), Sphingomonas (e.g. Sphingomonas paucimobilis), Stenotrophomonas (e.g. Stenotrophomonas maltophilia), Yersinia (e.g. Yersinia enterocolitica), and combinations thereof.

More particularly, the microorganism or group of microorganisms of interest may be selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Asaia siamensis, Lactobacillus casei, Lactobacillus plantarum, Weissella confusa, Clostridium sporogenes, Cutibacterium acnes, Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Listeria grayi, Shigella sonnei, Kocuria rhizophila and Burkholderia cepacia, Aeromonas hydrophila, Brevundimonas diminuta, Citrobacter freundii, Edwardsiella tarda, Enterobacter aerogenes, Ochrobactrum anthropic, Moraxella osloensis, Pantoea agglomerans, Proteus mirabilis, Ralstonia pickettii, Serratia marcescens, Sphingomonas paucimobilis, Stenotrophomonas maltophilia, Vibrio parahaemolyticus, Yersinia enterocolitica, and combinations thereof. Even more particularly, the microorganism or group of microorganisms of interest may be selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Asaia siamensis, Lactobacillus casei, Lactobacillus plantarum, Weissella confusa, Clostridium sporogenes, Cutibacterium acnes, Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Shigella sonnei, Kocuria rhizophila and Burkholderia cepacia, Aeromonas hydrophila, Brevundimonas diminuta, Citrobacter freundii, Edwardsiella tarda, Enterobacter aerogenes, Ochrobactrum anthropic, Moraxella osloensis, Pantoea agglomerans, Proteus mirabilis, Ralstonia pickettii, Serratia marcescens, Sphingomonas paucimobilis, Stenotrophomonas maltophilia, Yersinia enterocolitica, and combinations thereof.

Alternatively, the microorganism or group of microorganisms of interest may be selected from the group consisting of bacteria belonging to the genera Alicyclobacillus (e.g. Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans), Acetobacter (e.g. Acetobacter aceti), Gluconoacetobacter (e.g. Gluconoacetobacter liquefaciens), Gluconobacter (e.g. Gluconobacter oxydans), Asaia (e.g. Asaia siamensis), Lactobacillus (e.g. Lactobacillus casei, Lactobacillus plantarum), Weissella (e.g. Weissella confusa), Clostridium (e.g. Clostridium sporogenes), Cutibacterium (e.g Cutibacterium acnes), Methylobacterium (e.g. Methylobacterium extorquens), Pseudomonas (e.g. Pseudomonas aeruginosa), Bacillus (e.g. Bacillus subtilis), Escherichia (e.g. Escherichia coli), Staphylococcus (e.g. Staphylococcus aureus), Acinetobacter (e.g. Acinetobacter baumannii), Cronobacter (e.g. Cronobacter sakazakii), Klebsiella (e.g. Klebsiella pneumoniae), Salmonella (e.g. Salmonella typhimurium), Enterococcus (e.g. Enterococcus faecalis), Listeria (e.g. Listeria grayi), Shigella (e.g. Shigella sonnei), Kocuria (e.g. Kocuria rhizophila), Burkholderia (e.g. Burkholderia cepacia), and combinations thereof. The microorganism or group of microorganisms of interest may be also selected from the group consisting of bacteria belonging to the genera Alicyclobacillus (e.g. Alicyclobacillus acidoterrestris, Alicyclobacillus acidocaldarius, Alicyclobacillus acidiphilus, Alicyclobacillus cycloheptanicus, Alicyclobacillus hesperidum. Alicyclobacillus herbarius. Alicyclobacillus contaminans), Acetobacter (e.g. Acetobacter aceti), Gluconoacetobacter (e.g. Gluconoacetobacter liquefaciens), Gluconobacter (e.g. Gluconobacter oxydans), Asaia (e.g. Asaia siamensis), Lactobacillus (e.g. Lactobacillus casei, Lactobacillus plantarum), Weissella (e.g. Weissella confusa), Clostridium (e.g. Clostridium sporogenes), Cutibacterium (e.g Cutibacterium acnes), Methylobacterium (e.g. Methylobacterium extorquens), Pseudomonas (e.g. Pseudomonas aeruginosa), Bacillus (e.g. Bacillus subtilis), Escherichia (e.g. Escherichia coli), Staphylococcus (e.g. Staphylococcus aureus), Acinetobacter (e.g. Acinetobacter baumannii), Cronobacter (e.g. Cronobacter sakazakii), Klebsiella (e.g. Klebsiella pneumoniae), Salmonella (e.g. Salmonella typhimurium), Enterococcus (e.g. Enterococcus faecalis), Shigella (e.g. Shigella sonnei), Kocuria (e.g. Kocuria rhizophila), Burkholderia (e.g. Burkholderia cepacia), and combinations thereof. More particularly, the microorganism or group of microorganisms of interest may be selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Asaia siamensis, Lactobacillus casei, Lactobacillus plantarum, Weissella confusa, Clostridium sporogenes, Cutibacterium acnes, Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Listeria grayi, Shigella sonnei, Kocuria rhizophila and Burkholderia cepacia, and combinations thereof. The microorganism or group of microorganisms of interest may be also selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Asaia siamensis, Lactobacillus casei, Lactobacillus plantarum, Weissella confusa, Clostridium sporogenes, Cutibacterium acnes, Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Shigella sonnei, Kocuria rhizophila and Burkholderia cepacia, and combinations thereof.

In some other embodiments, the microorganism or group of microorganisms of interest are microscopic fungi, in particular yeasts or molds. Yeasts and molds may cause deterioration of various products such as foods, wines, beverages, cosmetic or pharmaceutical products. In particular, they can invade and grow on virtually any type of food at any time; they invade crops such as grains, nuts, beans, and fruits in fields before harvesting and during storage. They also grow on processed foods, food mixtures and are important spoilage organisms in dairy products, specifically in fermented dairy foods and low moisture products such as cheese. It has been estimated that 5-10% of food is wasted and must be thrown out due to fungal contamination. Preferably, the fungi to be detected or enumerated by the method of the invention belong to the division Ascomycota, and more preferably belong to the class Saccharomycetes or Deuteromycetes.

In an embodiment, the microorganism or group of microorganisms of interest is selected from the group consisting of fungi belonging to the genera Candida (e.g. Candida albicans), Zygosaccharomyces (e.g. Zygosaccharomyces bailii), Aspergillus (e.g. Aspergillus brasiliensis), Geotrichum (Geotrichum candidum), Saccharomyces (e.g. Saccharomyces cerevisiae) and Penicillium (e.g. Penicillium variotii, Penicillium chrysogenum), and combinations thereof. Preferably, the microorganism or group of microorganisms of interest is selected from the group consisting of Candida albicans, Zygosaccharomyces baili, Aspergillus brasiliensis, Saccharomyces cerevisiae, Geotrichum candidum, Penicillium variotii and Penicillium chrysogenum, and combinations thereof.

In particular, the microorganism or group of microorganisms of interest may be selected from the group consisting of bacteria belonging to the genera Alicyclobacillus (e.g. Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans), Acetobacter (e.g. Acetobacter aceti), Gluconoacetobacter (e.g. Gluconoacetobacter liquefaciens), Gluconobacter (e.g. Gluconobacter oxydans), Asaia (e.g. Asaia siamensis), Lactobacillus (e.g. Lactobacillus casei, Lactobacillus plantarum), Weissella (e.g. Weissella confusa), Clostridium (e.g. Clostridium sporogenes), Cutibacterium (e.g Cutibacterium acnes), Methylobacterium (e.g. Methylobacterium extorquens), Pseudomonas (e.g. Pseudomonas aeruginosa), Bacillus (e.g. Bacillus subtilis), Escherichia (e.g. Escherichia coli), Staphylococcus (e.g. Staphylococcus aureus), Acinetobacter (e.g. Acinetobacter baumannii), Cronobacter (e.g. Cronobacter sakazakii), Klebsiella (e.g. Klebsiella pneumoniae), Salmonella (e.g. Salmonella typhimurium), Enterococcus (e.g. Enterococcus faecalis), Listeria (e.g. Listeria grayi), Shigella (e.g. Shigella sonnei), Kocuria (e.g. Kocuria rhizophila), Burkholderia (e.g. Burkholderia cepacia), Aeromonas (e.g. Aeromonas hydrophila), Brevundimonas (e.g. Brevundimonas diminuta), Citrobacter (e.g. Citrobacter freundii), Edwardsiella (e.g. Edwardsiella tarda), Enterobacter (e.g. Enterobacter aerogenes), Ochrobactrum (e.g. Ochrobactrum anthropic), Moraxella (e.g. Moraxella osloensis), Pantoea (e.g. Pantoea agglomerans), Proteus (e.g. Proteus mirabilis), Ralstonia (e.g. Ralstonia pickettii), Serratia (e.g. Serratia marcescens), Sphingomonas (e.g. Sphingomonas paucimobilis), Stenotrophomonas (e.g. Stenotrophomonas maltophilia), Vibrio (e.g Vibrio parahaemolyticus), Yersinia (e.g. Yersinia enterocolitica), and fungi belonging to the genera Candida (e.g. Candida albicans), Zygosaccharomyces (e.g. Zygosaccharomyces bailii), Aspergillus (e.g. Aspergillus brasiliensis), Geotrichum (Geotrichum candidum), Saccharomyces (e.g. Saccharomyces cerevisiae) and Penicillium (e.g. Penicillium variotii, Penicillium chrysogenum), and combinations thereof. The microorganism or group of microorganisms of interest may also be selected from the group consisting of bacteria belonging to the genera Alicyclobacillus (e.g. Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans), Acetobacter (e.g. Acetobacter aceti), Gluconoacetobacter (e.g. Gluconoacetobacter liquefaciens), Gluconobacter (e.g. Gluconobacter oxydans), Asaia (e.g. Asaia siamensis), Lactobacillus (e.g. Lactobacillus casei, Lactobacillus plantarum), Weissella (e.g. Weissella confusa), Clostridium (e.g. Clostridium sporogenes), Cutibacterium (e.g Cutibacterium acnes), Methylobacterium (e.g. Methylobacterium extorquens), Pseudomonas (e.g. Pseudomonas aeruginosa), Bacillus (e.g. Bacillus subtilis), Escherichia (e.g. Escherichia coli), Staphylococcus (e.g. Staphylococcus aureus), Acinetobacter (e.g. Acinetobacter baumannii), Cronobacter (e.g. Cronobacter sakazakii), Klebsiella (e.g. Klebsiella pneumoniae), Salmonella (e.g. Salmonella typhimurium), Enterococcus (e.g. Enterococcus faecalis), Shigella (e.g. Shigella sonnei), Kocuria (e.g. Kocuria rhizophila), Burkholderia (e.g. Burkholderia cepacia), Aeromonas (e.g. Aeromonas hydrophila), Brevundimonas (e.g. Brevundimonas diminuta), Citrobacter (e.g. Citrobacter freundii), Edwardsiella (e.g. Edwardsiella tarda), Enterobacter (e.g. Enterobacter aerogenes), Ochrobactrum (e.g. Ochrobactrum anthropic), Moraxella (e.g. Moraxella osloensis), Pantoea (e.g. Pantoea agglomerans), Proteus (e.g. Proteus mirabilis), Ralstonia (e.g. Ralstonia pickettii), Serratia (e.g. Serratia marcescens), Sphingomonas (e.g. Sphingomonas paucimobilis), Stenotrophomonas (e.g. Stenotrophomonas maltophilia), Yersinia (e.g. Yersinia enterocolitica), and fungi belonging to the genera Candida (e.g. Candida albicans), Zygosaccharomyces (e.g. Zygosaccharomyces bailii), Aspergillus (e.g. Aspergillus brasiliensis), Geotrichum (Geotrichum candidum), Saccharomyces (e.g. Saccharomyces cerevisiae) and Penicillium (e.g. Penicillium variotii, Penicillium chrysogenum), and combinations thereof.

More particularly, the microorganism or group of microorganisms of interest may be selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Asaia siamensis, Lactobacillus casei, Lactobacillus plantarum, Weissella confusa, Clostridium sporogenes, Cutibacterium acnes, Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Listeria grayi, Shigella sonnei, Kocuria rhizophila, Burkholderia cepacia, Aeromonas hydrophila, Brevundimonas diminuta, Citrobacter freundii, Edwardsiella tarda, Enterobacter aerogenes, Ochrobactrum anthropic, Moraxella osloensis, Pantoea agglomerans, Proteus mirabilis, Ralstonia pickettii, Serratia marcescens, Sphingomonas paucimobilis, Stenotrophomonas maltophilia, Vibrio parahaemolyticus, Yersinia enterocolitica, Candida albicans, Zygosaccharomyces bailii, Aspergillus brasiliensis, Saccharomyces cerevisiae, Geotrichum candidum, Penicillium variotii and Penicillium chrysogenum, and combinations thereof. The microorganism or group of microorganisms of interest may also be selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Asaia siamensis, Lactobacillus casei, Lactobacillus plantarum, Weissella confusa, Clostridium sporogenes, Cutibacterium acnes, Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Shigella sonnei, Kocuria rhizophila, Burkholderia cepacia, Aeromonas hydrophila, Brevundimonas diminuta, Citrobacter freundii, Edwardsiella tarda, Enterobacter aerogenes, Ochrobactrum anthropic, Moraxella osloensis, Pantoea agglomerans, Proteus mirabilis, Ralstonia pickettii, Serratia marcescens, Sphingomonas paucimobilis, Stenotrophomonas maltophilia, Yersinia enterocolitica, Candida albicans, Zygosaccharomyces bailii, Aspergillus brasiliensis, Saccharomyces cerevisiae, Geotrichum candidum, Penicillium variotii and Penicillium chrysogenum, and combinations thereof.

Alternatively, the microorganism or group of microorganisms of interest may be selected from the group consisting of bacteria belonging to the genera Alicyclobacillus (e.g. Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans), Acetobacter (e.g. Acetobacter aceti), Gluconoacetobacter (e.g. Gluconoacetobacter liquefaciens), Gluconobacter (e.g. Gluconobacter oxydans), Asaia (e.g. Asaia siamensis), Lactobacillus (e.g. Lactobacillus casei, Lactobacillus plantarum), Weissella (e.g. Weissella confusa), Clostridium (e.g. Clostridium sporogenes), Cutibacterium (e.g Cutibacterium acnes), Methylobacterium (e.g. Methylobacterium extorquens), Pseudomonas (e.g. Pseudomonas aeruginosa), Bacillus (e.g. Bacillus subtilis), Escherichia (e.g. Escherichia coli), Staphylococcus (e.g. Staphylococcus aureus), Acinetobacter (e.g. Acinetobacter baumannii), Cronobacter (e.g. Cronobacter sakazakii), Klebsiella (e.g. Klebsiella pneumoniae), Salmonella (e.g. Salmonella typhimurium), Enterococcus (e.g. Enterococcus faecalis), Listeria (e.g. Listeria grayi), Shigella (e.g. Shigella sonnei), Kocuria (e.g. Kocuria rhizophila), Burkholderia (e.g. Burkholderia cepacia), and fungi belonging to the genera Candida (e.g. Candida albicans), Zygosaccharomyces (e.g. Zygosaccharomyces bailii), Aspergillus (e.g. Aspergillus brasiliensis), Geotrichum (Geotrichum candidum), Saccharomyces (e.g. Saccharomyces cerevisiae) and Penicillium (e.g. Penicillium variotii, Penicillium chrysogenum), and combinations thereof. The microorganism or group of microorganisms of interest may also be selected from the group consisting of bacteria belonging to the genera Alicyclobacillus (e.g. Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans), Acetobacter (e.g. Acetobacter aceti), Gluconoacetobacter (e.g. Gluconoacetobacter liquefaciens), Gluconobacter (e.g. Gluconobacter oxydans), Asaia (e.g. Asaia siamensis), Lactobacillus (e.g. Lactobacillus casei, Lactobacillus plantarum), Weissella (e.g. Weissella confusa), Clostridium (e.g. Clostridium sporogenes), Cutibacterium (e.g Cutibacterium acnes), Methylobacterium (e.g. Methylobacterium extorquens), Pseudomonas (e.g. Pseudomonas aeruginosa), Bacillus (e.g. Bacillus subtilis), Escherichia (e.g. Escherichia coli), Staphylococcus (e.g. Staphylococcus aureus), Acinetobacter (e.g. Acinetobacter baumannii), Cronobacter (e.g. Cronobacter sakazakii), Klebsiella (e.g. Klebsiella pneumoniae), Salmonella (e.g. Salmonella typhimurium), Enterococcus (e.g. Enterococcus faecalis), Shigella (e.g. Shigella sonnei), Kocuria (e.g. Kocuria rhizophila), Burkholderia (e.g. Burkholderia cepacia), and fungi belonging to the genera Candida (e.g. Candida albicans), Zygosaccharomyces (e.g. Zygosaccharomyces bailii), Aspergillus (e.g. Aspergillus brasiliensis), Geotrichum (Geotrichum candidum), Saccharomyces (e.g. Saccharomyces cerevisiae) and Penicillium (e.g. Penicillium variotii, Penicillium chrysogenum), and combinations thereof. More particularly, the microorganism or group of microorganisms of interest may be selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Asaia siamensis, Lactobacillus casei, Lactobacillus plantarum, Weissella confusa, Clostridium sporogenes, Cutibacterium acnes, Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Listeria grayi, Shigella sonnei, Kocuria rhizophila, Burkholderia cepacia, Candida albicans, Zygosaccharomyces baili, Aspergillus brasiliensis, Saccharomyces cerevisiae, Geotrichum candidum, Penicillium variotii and Penicillium chrysogenum, and combinations thereof. The microorganism or group of microorganisms of interest may also be selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Asaia siamensis, Lactobacillus casei, Lactobacillus plantarum, Weissella confusa, Clostridium sporogenes, Cutibacterium acnes, Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Shigella sonnei, Kocuria rhizophila, Burkholderia cepacia, Candida albicans, Zygosaccharomyces bailii, Aspergillus brasiliensis, Saccharomyces cerevisiae, Geotrichum candidum, Penicillium variotii and Penicillium chrysogenum, and combinations thereof.

In preferred embodiments, the method of the invention is used to detect or enumerate a group of microorganisms of interest.

In particular, the group of microorganisms may comprises
two or more microorganisms selected from the group of thermophilic acidophilic bacteria (TAB) as defined above, i.e. belonging to the Alicyclobacillus genus, preferably two or more microorganisms selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius and Alicyclobacillus contaminans, and combination thereof; or
two or more microorganisms selected from the group of acetic acid bacteria (AAB) as defined above, preferably selected from bacteria belonging to the genera Acetobacter, Acidomonas, Asaia, Gluconacetobacter, Gluconobacter, Ameyamaea, Bombella, Commensalibacter, Endobacter, Granulibacter, Komagataeibacter, Kozakia, Neoasaia, Neokomagataea, Nguyenibacter, Saccharibacter, Swaminathania, Swingsia and Tanticharoenia, and combination thereof, more preferably selected from bacteria belonging to the genera Acetobacter, Gluconobacter, Gluconacetobacter and Asaia, and combination thereof, and even more preferably selected from the group consisting of Acetobacter aceti, Gluconoacetobacter liquefaciens, Gluconobacter oxydans and Asaia siamensis, and combination thereof; or
two or more microorganisms selected from the group of lactic acid bacteria (LAB) as defined above, preferably selected from bacteria belonging to the genera Lactobacillus, Leuconostoc, Pediococcus, Lactococcus, Enterococcus, Carnobacterium, Streptococcus and Weissella, and combination thereof, more preferably selected from bacteria belonging to the genera Lactobacillus and Weissella, and combination thereof, and even more preferably selected from the group consisting of Lactobacillus casei, Lactobacillus plantarum and Weissella confusa, and combination thereof; or
two or more microorganisms selected from the group of anaerobic bacteria, preferably selected from Clostridium and Cutibacterium bacteria, and combination thereof, more preferably, selected from the group consisting of Clostridium sporogenes and Cutibacterium acnes, and combination thereof; or
two or more microorganisms selected from the group of aerobic mesophilic bacteria (AMB) as defined above, preferably selected from bacteria belonging to the genera Methylobacterium, Pseudomonas, Bacillus, Escherichia, Staphylococcus, Acinetobacter, Cronobacter, Klebsiella, Salmonella, Enterococcus, Listeria, Shigella, Kocuria and Burkholderia, and combination thereof, more preferably, selected from the group consisting of Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Listeria grayi, Shigella sonnei, Kocuria rhizophila and Burkholderia cepacia, and combination thereof, even more preferably selected from the group consisting of Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Shigella sonnei, Kocuria rhizophila and Burkholderia cepacia, and combination thereof; or
two or more microorganisms selected from the group of Gram negative bacteria as defined above, preferably selected from bacteria belonging to the genera Escherichia, Proteus and Pseudomonas, and combination thereof, more preferably selected from Escherichia coli, Proteus mirabilis and Pseudomonas aeruginosa, and combination thereof, even more preferably from bacteria belonging to the genera Escherichia and Pseudomonas, and combination thereof, in particular selected from Escherichia coli and Pseudomonas aeruginosa, and combination thereof; or
two or more microorganisms selected from the group of Gram positive bacteria as defined above, preferably selected from bacteria belonging to the genera Staphylococcus, Enterococcus, Streptococcus, and combination thereof, more preferably selected from Staphylococcus aureus and Enterococcus faecalis, and combination thereof; or
two or more microorganisms selected from the group of heterotrophic bacteria as defined above, preferably selected from bacteria belonging to the genera Acinetobacter, Aeromonas, Brevundimonas, Burkholderia, Citrobacter, Edwardsiella, Enterobacter, Escherichia, Ochrobactrum, Klebsiella, Methylobacterium, Moraxella, Pantoea, Proteus, Pseudomonas, Ralstonia, Salmonella, Serratia, Shigella, Sphingomonas, Stenotrophomonas, Vibrio, Yersinia, Bacillus, Enterococcus, Micrococcus, Staphylococcus, and combinations thereof, more preferably selected from Acinetobacter baumanii, Aeromonas hydrophila, Brevundimonas diminuta, Burkholderia cepacia, Citrobacter freundii, Edwardsiella tarda, Enterobacter aerogenes, Escherichia coli, Ochrobactrum anthropic, Klebsiella pneumoniae, Methylobacterium extorquens, Moraxella osloensis, Pantoea agglomerans, Proteus mirabilis, Pseudomonas aeruginosa, Pseudomonas fluorescens, Ralstonia pickettii, Salmonella typhimurium, Serratia marcescens, Shigella sonnei, Sphingomonas paucimobilis, Stenotrophomonas maltophilia, Vibrio parahaemolyticus, Yersinia enterocolitica, Bacillus subtilis, Enterococcus faecalis, Micrococcus luteus (Kocuria rhizophila), Staphylococcus aureus, and combinations thereof, even more preferably selected from Acinetobacter baumanii, Aeromonas hydrophila, Brevundimonas diminuta, Burkholderia cepacia, Citrobacter freundii, Edwardsiella tarda, Enterobacter aerogenes, Escherichia coli, Ochrobactrum anthropic, Klebsiella pneumoniae, Methylobacterium extorquens, Moraxella osloensis, Pantoea agglomerans, Proteus mirabilis, Pseudomonas aeruginosa, Pseudomonas fluorescens, Ralstonia pickettii, Salmonella typhimurium, Serratia marcescens, Shigella sonnei, Sphingomonas paucimobilis, Stenotrophomonas maltophilia, Yersinia enterocolitica, Bacillus subtilis, Enterococcus faecalis, Micrococcus luteus (Kocuria rhizophila), Staphylococcus aureus, and combinations thereof; or
two or more microorganisms selected from the groups, as defined above, of thermophilic acidophilic bacteria (TAB), acetic acid bacteria (AAB), lactic acid bacteria (LAB), anaerobic bacteria, aerobic mesophilic bacteria, Gram negative bacteria and Gram positive bacteria, heterotrophic bacteria, and combinations thereof, preferably selected from the groups, as defined above, of thermophilic acidophilic bacteria (TAB), acetic acid bacteria (AAB), lactic acid bacteria (LAB), anaerobic bacteria, aerobic mesophilic bacteria, Gram negative bacteria and Gram positive bacteria, and combinations thereof; or
two or more microorganisms selected from yeasts and molds as defined above, preferably selected from fungi belonging to the genera Candida, Zygosaccharomyces, Aspergillus, Saccharomyces, Geotrichum and Penicillium, and combinations thereof, more preferably selected from the group consisting of Candida albicans, Zygosaccharomyces baili, Aspergillus brasiliensis, Saccharomyces cerevisiae, Geotrichum candidum, Penicillium variotii, Penicillium chrysogenum, and combinations thereof; or
two or more microorganisms selected from the groups, as defined above, of thermophilic acidophilic bacteria (TAB), acetic acid bacteria (AAB), lactic acid bacteria (LAB), anaerobic bacteria, aerobic mesophilic bacteria, Gram negative bacteria and Gram positive bacteria, heterotrophic bacteria, yeasts and molds, and combinations thereof, preferably selected from the groups, as defined above, of thermophilic acidophilic bacteria (TAB), acetic acid bacteria (AAB), lactic acid bacteria (LAB), anaerobic bacteria, aerobic mesophilic bacteria, Gram negative bacteria and Gram positive bacteria, yeasts and molds, and combinations thereof.

In particular, the group of microorganisms to be detected or enumerated by the method of the invention may comprise
one or several bacteria, preferably selected from the groups, as defined above, of thermophilic acidophilic bacteria (TAB), acetic acid bacteria (AAB), lactic acid bacteria (LAB), anaerobic bacteria, aerobic mesophilic bacteria, Gram negative bacteria and Gram positive bacteria, heterotrophic bacteria, and combinations thereof, and
one or several microscopic fungi, preferably selected from yeasts and molds as defined above.

Preferably, the group of microorganisms to be detected or enumerated by the method of the invention may comprise
one or several bacteria, preferably selected from the groups, as defined above, of thermophilic acidophilic bacteria (TAB), acetic acid bacteria (AAB), lactic acid bacteria (LAB), anaerobic bacteria, aerobic mesophilic bacteria, Gram negative bacteria and Gram positive bacteria, and combinations thereof, and
one or several microscopic fungi, preferably selected from yeasts and molds as defined above.

The microorganism or group of microorganisms to be detected or enumerated by the method of the invention may depend on the nature of the sample.

For example:
Samples obtained from fruit juices, juice related products or syrups, might be suspected of containing thermophilic acidophilic bacteria;
Samples obtained from vacuum packaged foods or canned foods might be suspected of containing anaerobic bacteria;
Samples obtained from pharmaceutical raw materials might be suspected of containing mesophilic aerobic bacteria, in particular from Bacillus genus, anaerobic bacteria, in particular from Clostridium genus, or fungi such as Penicillium and Aspergillus fungi;
Samples obtained from cosmetic products might be suspected of containing aerobic mesophilic bacteria, in particular P. aeruginosa, S. aureus, E. coli, and Bacillus species; and
Samples obtained from medical devices or urine (i.e. for UTI diagnosis) might be suspected of containing Gram-negative bacteria, in particular E. coli, Pseudomonas aeruginosa, Acinetobacter baumanii, Proteus mirabilis or Klebsiella pneumoniae, or Gram-positive bacteria, in particular Staphylococcus aureus or Enterococcus faecalis; and
Samples obtained from pharmaceutical water systems might be suspected of containing heterotrophic bacteria as defined above, in particular selected from bacteria belonging to the genera Acinetobacter, Aeromonas, Brevundimonas, Burkholderia, Citrobacter, Edwardsiella, Enterobacter, Escherichia, Ochrobactrum, Klebsiella, Methylobacterium, Moraxella, Pantoea, Proteus, Pseudomonas, Ralstonia, Salmonella, Serratia, Shigella, Sphingomonas, Stenotrophomonas, Vibrio, Yersinia, Bacillus, Enterococcus, Micrococcus, Staphylococcus, and combinations thereof, preferably selected from bacteria belonging to the genera Acinetobacter, Aeromonas, Brevundimonas, Burkholderia, Citrobacter, Edwardsiella, Enterobacter, Escherichia, Ochrobactrum, Klebsiella, Methylobacterium, Moraxella, Pantoea, Proteus, Pseudomonas, Ralstonia, Salmonella, Serratia, Shigella, Sphingomonas, Stenotrophomonas, Yersinia, Bacillus, Enterococcus, Micrococcus, Staphylococcus, and combinations thereof, more preferably selected from Acinetobacter baumanii, Aeromonas hydrophila, Brevundimonas diminuta, Burkholderia cepacia, Citrobacter freundii, Edwardsiella tarda, Enterobacter aerogenes, Escherichia coli, Ochrobactrum anthropic, Klebsiella pneumoniae, Methylobacterium extorquens, Moraxella osloensis, Pantoea agglomerans, Proteus mirabilis, Pseudomonas aeruginosa, Pseudomonas fluorescens, Ralstonia pickettii, Salmonella typhimurium, Serratia marcescens, Shigella sonnei, Sphingomonas paucimobilis, Stenotrophomonas maltophilia, Vibrio parahaemolyticus, Yersinia enterocolitica, Bacillus subtilis, Enterococcus faecalis, Micrococcus luteus (Kocuria rhizophila), Staphylococcus aureus, and combinations thereof, even more preferably selected from Acinetobacter baumanii, Aeromonas hydrophila, Brevundimonas diminuta, Burkholderia cepacia, Citrobacter freundii, Edwardsiella tarda, Enterobacter aerogenes, Escherichia coli, Ochrobactrum anthropic, Klebsiella pneumoniae, Methylobacterium extorquens, Moraxella osloensis, Pantoea agglomerans, Proteus mirabilis, Pseudomonas aeruginosa, Pseudomonas fluorescens, Ralstonia pickettii, Salmonella typhimurium, Serratia marcescens, Shigella sonnei, Sphingomonas paucimobilis, Stenotrophomonas maltophilia, Yersinia enterocolitica, Bacillus subtilis, Enterococcus faecalis, Micrococcus luteus (Kocuria rhizophila), Staphylococcus aureus, and combinations thereof.

For each domain wherein the method of the invention can be used (e.g. pharmaceuticals, cosmetics, foods and beverages, environmental analysis, diagnosis etc.), the skilled person knows which microorganisms can be suspected to be contained in the sample and can thus adapt the method of the invention to these microorganisms.

### Growth Medium

In step a) of the method of the invention, the sample is contacted, in a container, with a solid growth medium containing nutrients to support growth of the microorganism or group of microorganisms of interest.

As used herein, the term “growth medium” or “culture medium” refers to a nutrient medium used for growth of microorganism or group of microorganisms within the context of the present invention. The growth medium can be a defined medium synthesized from individual chemicals so the exact molecular composition is known, or an undefined medium comprising some complex ingredients, such as yeast extract or casein hydrolysate, which consist of a mixture of many chemical species in unknown proportions.

The growth medium used in the method of the invention may be a non-selective growth medium or a selective growth medium. A non-selective growth medium is a general media for bacterial growth, such as Tryptone Soy Agar (TSA), for fungi growth, such as Sabouraud Dextrose Agar (SDA) or for both bacterial and fungi growth, such as Reasoner's 2A Agar (R2A). A non-selective medium typically contains the nutrients required to support the growth of a wide variety of microorganisms. A selective growth medium is a medium used for the growth of only selected microorganisms. Indeed, the composition of a selective medium ensures the proliferation of cells with certain properties, such as antibiotic resistance or the ability to synthesize a certain metabolite, e.g. an amino acid. This medium thus provides an environment that favors the growth of the target microorganisms over nontarget microorganisms that may be present in the sample.

The expression “solid growth medium” or “solid culture medium” as used herein refers to a growth medium which allows microorganisms to form microcolonies on its surface, such as a medium which has a gel-like appearance or is in the form of a gel, a gel being a colloidal system in which a porous network of interconnected particles spans the volume of a liquid medium and allows nutrients to diffuse through the medium to become available to the microorganisms. Preferably, the solid growth medium as used herein is prepared by adding to a liquid growth medium a sufficient amount of a gelling agent such as agar, agarose, alginate, carrageenan, cellulose, gelatin, pectin and combinations thereof. Typically, the solid growth medium contains a gelling agent, preferably agar, at a concentration of 0.5% to 3%, preferably 1% to 2.5%. Preferably, the solid growth medium used in the method of the invention is an agar growth medium.

The solid growth medium is poured in a container. This contained can be any sterile container adapted to microbial culture, in particular a conventional Petri plate.

The choice of the growth medium essentially depends on the microorganism or group of microorganisms of interest. However, this choice may also depend on the application domain (e.g. pharmaceuticals, cosmetics, foods and beverages, environmental analysis, diagnosis etc.) and standard methods used in this domain. As illustration, to simultaneously detect yeasts, molds, lactic acid bacteria and acetic acid bacteria from a sample obtained from non-alcoholic beverages, IFU Microbiological Method 2 recommends Orange serum agar (OSA) medium. On the other hand, for bioburden testing in pharmaceutical industry, R2A or TSA medium is recommended. Similarly, some applications require selective media to detect only a particular microorganism or a particular group of microorganisms (e.g. MacConkey agar medium for analysis of a urine sample to diagnose UTI caused by Gram negative bacteria), while other applications such as sterility testing or bioburden testing typically require non selective media in order to detect or enumerate as much as possible microorganisms. Of course, as explained thereafter, conditions and time of incubation also play a role in the selectivity or non-selectivity of the method. The skilled person can easily take into account these parameters to choose the suitable growth medium.

Examples of growth media suitable to support growth of thermophilic acidophilic bacteria include, but are not limited to, YSG (Yeast-Starch-Glucose) agar (Matsubara et al. 2002, Int J Syst Evol Microbiol 52, 1681-1685), PDA (Potato Dextrose Agar; Pettipher et al. 1997, Lett Appl Microbiol 24, 185-189), OSA (Orange Serum Agar; Pettipher et al. 2000, Food Austr 57, 293-295), K agar (Walls et al. 1998, Dairy Food Environ Sanit 18, 499-503), BAT (Bacillus acidoterrestris) agar. Preferably, the growth medium used to support growth of thermophilic acidophilic bacteria is a growth medium recommended for the detection of thermophilic acidophilic bacteria according to IFU Standard Method No. 12.

Examples of growth media suitable to support growth of lactic acid bacteria include, but are not limited to, Rogosa agar (Rogosa et al. 1951, J Bacteriol 62, 132-133), MRS (De Man, Rogosa and Sharpe) agar medium (De Man et al. 1960, J Appl Bacteriol 23, 30-35), Lactobacilli selective agar (Mitsuoka, 1978, Intestinal Bacteria and Health. Tokyo, Harcourt Brace Jovanovich Japan), LAMVAB (Hartemink et al. 1997, J Microbiol Methods 29, 77-84), TSA-BCP and PCA-BCP (Trypticase Soy Agar and Plate Count Agar with BromoCresol Purple; Lin et al. 2006, Food Microbiol 23, 74-81) or Raka Ray agar. Preferably, the growth medium used to support growth of lactic acid bacteria is a growth medium recommended for the detection of lactic acid bacteria according IFU Standard Method No. 5.

Examples of growth media suitable to support growth of yeasts or molds include, but are not limited to, YM (Yeast extract Malt extract) and YMG (Yeast extract Malt extract Glucose) agar media (Wickerham, 1939 J. Tropical Med. Hyg., 42, 176), OSA (Orange Serum Agar), PDA (Potato Dextrose Agar), Tomato juice agar (MacFaddin J. F., 1985, Media for Isolation-Cultivation-Identification-Maintenance of Medical Bacteria, Vol. 1, Williams & Wilkins, Baltimore, Md), MEA (Malt Extract agar) medium, Glucose peptone agar (Glathe et al., Zentralbl Bakteriol Parasitenkd Infektionskr Hyg. 1968; 122(1):3-21), Wallerstein nutrient agar (Green and Gray. 1950. Wallerstein Lab. Commun. 12:43), Wort agar (Parfitt E. H. (1933) J. Dairy Sci. 16. 141-147) and Sabouraud Dextrose Agar (Sabouraud, R. (1892) Ann. Dermatol. Syphilol. 3:1061). In particular, YM and YMG agar media are selective growth media with low pH useful for cultivating yeasts, molds, or other acid-tolerant or acidophilic organisms (i.e. capable of growth in acidic natural environments having pH ranges from 3 to 4), while deterring growth of most bacteria and other acid intolerant organisms. Preferably, the growth medium used to support growth of yeasts or molds is a growth medium recommended for the detection of yeasts or molds according to USP61 standard, BS EN ISO 16212-2017 standard or IFU Standard Method No. 3 and 4.

Examples of growth media suitable to support growth of acetic acid bacteria include, but are not limited to, YM (Yeast extract Malt extract) agar medium, YMG (Yeast extract Malt extract Glucose) agar medium, GYC (Glucose Yeast extract Calcium carbonate; 10% glucose, 1.0% yeast extract, 2.0% calcium carbonate, 1.5% agar, pH 6.8) agar medium, YPE (yeast extract-peptone-ethanol) agar medium, YPG (Yeast extract Polypeptone Glycerol), YPM/YPS (Yeast extract Polypeptone Mannitol or Sorbitol) agar medium, YPGD (Yeast extract Polypeptone Glucose) agar medium.

Examples of growth media suitable to support growth of aerobic mesophilic bacteria include, but are not limited to, TSA (Tryptone Soy Agar) medium, PCA (Plate Count Agar) medium, R2A (Reasoner's 2A agar) medium, SCDA (Soybean Casein Digest Agar), FTM (Fluid Thioglycollate Medium) agar medium. In particular, TSA and PCA media are non-selective growth media commonly used to assess viable bacterial growth of a sample and R2A medium is a culture medium adapted to the growth of bacteria which normally inhabit drinking water. Preferably, the growth medium used to support growth of aerobic mesophilic bacteria is a growth medium recommended for the detection of aerobic mesophilic bacteria according to USP61 standard or BS EN ISO 21149-2017 standard.

Examples of growth media suitable to support growth of anaerobic bacteria include, but are not limited to, TSA (Tryptone Soy Agar) medium, Reinforced clostridial agar (Barnes et al. (1963) J. Appl. Bact. 26. 415-427), Anaerobic agar (Brewer J. H., 1942, Science, 95:587), Columbia agar (Ellner et al. Amer. J. Clin. Path., 29; 181-183 (1958)) optionally supplemented with defibrinated sheep blood. In some embodiments, the growth medium used to support growth of anaerobic bacteria may be a growth medium recommended for the detection of anaerobic bacteria, and in particular for the detection of Clostridium perfringens according to ISO 7937:2004 or ISO 14189:2013 standards.

Examples of growth media suitable to support growth of Gram positive bacteria include, but are not limited to, MSA (Mannitol salt agar) medium or Phenylethyl Alcohol Agar (Brewer and Lilley. 1949. December meeting of the Maryland Association of Medical and Public Health Laboratories).

Examples of growth media suitable to support growth of Gram negative bacteria include, but are not limited to, MacConkey agar medium, Hektoen enteric agar (King and Metzger. 1968. Appl. Microbiol. 16:577-578) or XLD (Xylose-Lysine-Desoxycholate) Agar (Taylor, 1965. Am. J. Clin. Pathol., 44:471-475). In particular, MacConkey agar medium contains bile salts and crystal violet, which interfere with the growth of many gram-positive bacteria and favor the growth of gram-negative bacteria, particularly the Enterobacteriaceae. In some embodiments, the growth medium used to support growth of Gram negative bacteria may be a growth medium recommended for the detection of Gram negative bacteria belonging to the Enterobacteriaceae family, according to ISO 21528:2017 standard.

Examples of growth media suitable to support growth of bacteria and fungi include, but are not limited to, Reasoner's 2A Agar (R2A), Tryptone Yeast Extract Agar or Orange serum agar. Preferably, the growth medium used to support growth of all microorganisms (bacteria, yeasts and molds) is a growth medium recommended for the enumeration of culturable micro-organisms (total count) according to IFU Standard Method No. 2 or ISO 6222:1999 standard.

Optionally, in order to increase the selectivity of a medium, one or several additional selective components can be added to the medium such as antibiotics, pH indicators, selective growth inhibitors.

All of these growth media are well known by the skilled person and are commercially available or easily prepared.

### Fluorescent Dye

In step a) of the method of the invention, the sample is contacted not only with a solid growth medium as described above but also with at least one fluorescent dye.

As used herein, the terms “fluorescent dye”, “fluorophore” or “fluorochrome” are used interchangeably and refer to a chemical compound that can re-emit light upon light excitation. Typically, fluorophores absorb light of certain wavelength range (excitation spectrum) and re-emitting it at a longer wavelength range (emission spectrum) with respective excitation and emission maxima. This term refers to a chemical compound that does not require any chemical modification to exhibit fluorescent property. In particular, said at least one fluorescent dye is not a fluorogenic substrate, i.e. a substrate which produces a fluorescent substance after a reaction with an enzyme or a group of enzymes of a microorganism. In addition, said at least one fluorescent dye is not linked to a functional moiety which is able to specifically recognize a microorganism or a group of microorganisms, such as an antibody. Preferably, the fluorescent dyes used in the method of the invention do not restrict growth of microorganisms of interest, i.e. the growth of the microorganisms of interest is not significantly impacted by the presence of the fluorescent dye(s). Preferably, said at least one fluorescent dye is not encapsulated, e.g. in liposomes or lipid vesicles. Preferably, said at least one fluorescent dye is not a DNA and/or RNA intercalating agent. Preferably, said at least one fluorescent dye is not sulforhodamine B, a Hoechst dye, rhodamine B, neutral red, bromothymol blue, eosin, methylene blue, amphotericin B nor Nile Red.

In the method of the invention, the sample may be contacted with one or several fluorescent dyes. Preferably, each fluorescent dye used in this method is able to stain at least one microorganism of interest. In some embodiments, several fluorescent dyes are used, each fluorescent dye being able to stain one or several of microorganisms of interest and all microorganisms of interest being stained by at least one of these fluorescent dyes. The fluorescent dyes used in the method of the invention may be membrane-permeable fluorescent dyes and stain the inner side of the microorganisms or may be just adsorbed on the outer surface of the microorganisms.

The skilled person may choose one or several fluorescent dyes depending on the microorganism or group of microorganisms to be detected.

In particular, the skilled person may assess the capacity of a fluorescent dye to stain a microorganism of interest as described in the experimental section. Briefly, a culture of said microorganism is filtered on 0.45 μm a membrane filter, e.g. MCE or PVDF membrane, and said filtration membrane is deposited above a drop of the fluorescent dye to be tested on a solid growth medium suitable to support growth of said microorganism. Said medium is incubated under conditions and for a time sufficient to form microcolonies of said microorganism. Detection of microcolonies emitting a fluorescent signal is indicative that the fluorescent dye can be used in the method of the invention to detect said microorganism. If a group of microorganisms has to be detected, the skilled person may repeat this assay for each microorganism of this group, with the same or with different fluorescent dyes until determining the suitable combination allowing detection of all microorganisms of interest.

In an embodiment, the sample is contacted with at least one fluorescent dye selected from the group consisting of acridine dyes such as acridine orange, anthracene-based dyes such as anthraquinone dyes, coumarin-based dyes, cyanine-based dyes such as cyanine, indocarbocyanine, oxacarbocyanine, thiacarbocyanine and merocyanine dyes, dipyrromethene-based dyes, naphthalene-based dyes, pyrene-based dyes, xanthene-based dyes such as fluorescein dyes, eosin dyes, rhodamine dyes and carbopyronines dyes, squaraine-based dyes, squaraine rotaxane-based dyes and 4′,6-diamidino-2-phenylindole (DAPI), and combinations thereof. Preferably, the sample is contacted with at least one fluorescent dye selected from the group consisting of anthracene-based dyes such as anthraquinone dyes, coumarin-based dyes, cyanine-based dyes such as cyanine, indocarbocyanine, oxacarbocyanine, thiacarbocyanine and merocyanine dyes, dipyrromethene-based dyes, naphthalene-based dyes, pyrene-based dyes, xanthene-based dyes such as fluorescein dyes, eosin dyes, rhodamine dyes and carbopyronines dyes, squaraine-based dyes, squaraine rotaxane-based dyes and 4′,6-diamidino-2-phenylindole (DAPI), and combinations thereof.

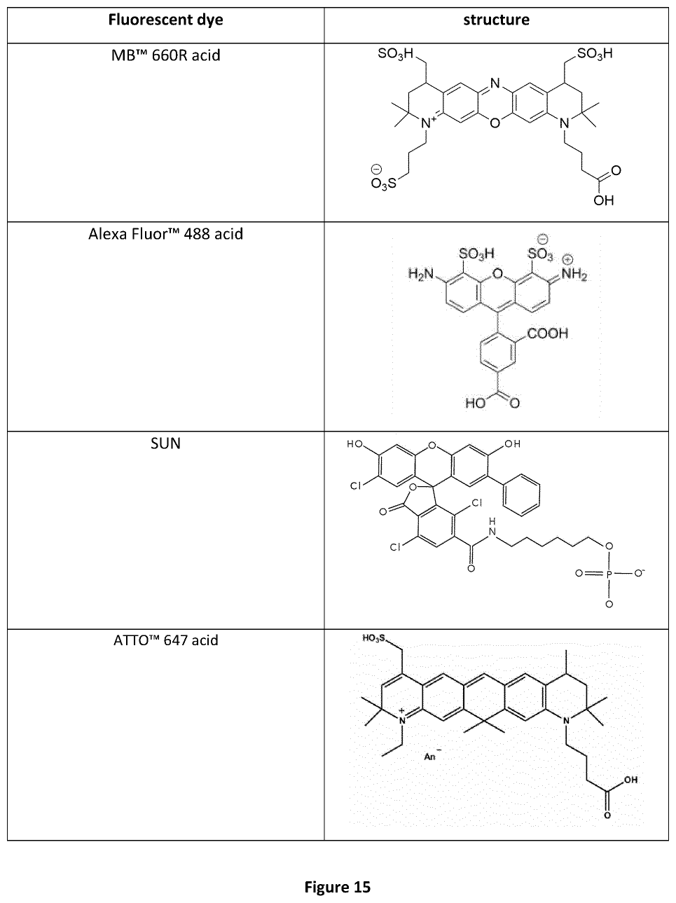

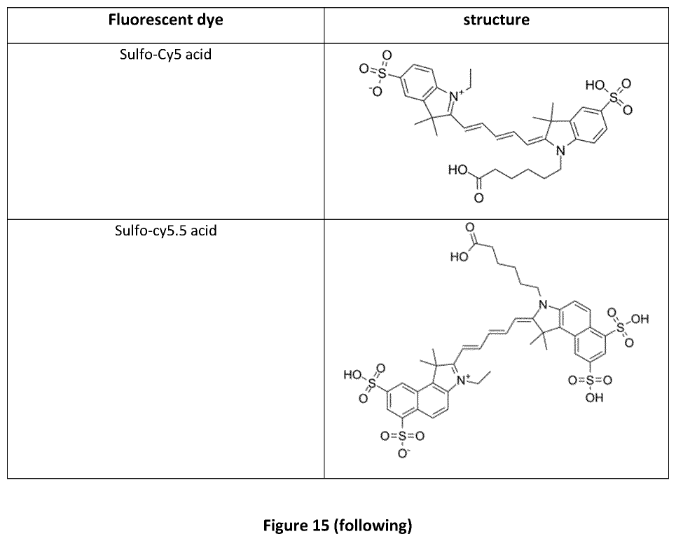

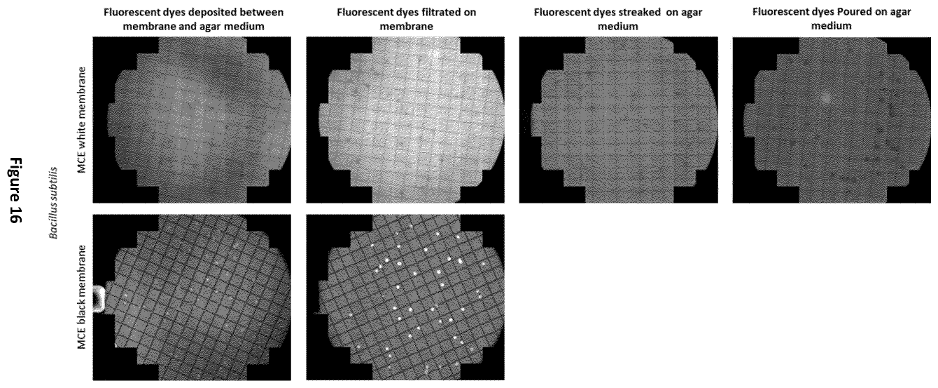

In particular, said at least one fluorescent dye may be selected from the group consisting of
acridine dyes, in particular acridine orange,
anthracene-based dyes, in particular DRAQ5™ (e.g. 1,5-bis (2-(di-methylamino)ethylamino)-4,8-dihydroxyanthracene-9,10-dione), CyTRAK Orange™ (Edward, R. (2009) Mol. Cells 27(4): 391-396; CAS Registry Number 1195771-25-5) and derivatives thereof,
coumarin-based dyes, in particular Alexa Fluor™ 350 (e.g. 7-amino-3-[2-(2,5-dioxopyrrolidin-1-yl)oxy-2-oxoethyl]-4-methyl-2-oxochromene-6-sulfonic acid), Pacific Blue™ (e.g. 6,8-difluoro-7-hydroxy-2-oxochromene-3-carboxylic acid) and derivatives thereof,
Cyanine-based dyes, in particular Cy5™ or Cy5.5™, including sulfonated and non-sulfonated forms, preferably sulfonated forms such as sulfonated Cy5™ (e.g. sulfo-Cy5 acid, FIG. 15) or sulfonated Cy5.5™ (e.g. Sulfo-Cy5.5 acid, FIG. 15), indocyanine green (e.g. sodium; 4-[(2Z)-2-[(2E,4E,6E)-7-[1,1-dimethyl-3-(4-sulfonatobutyl)benzo[e]indol-3-ium-2-yl]hepta-2,4,6-trienylidene]-1,1-dimethylbenzo[e]indol-3-yl]butane-1-sulfonate) and derivatives thereof,
dipyrromethene-based dyes, in particular BODIPY™ dyes (based on the core structure 4,4-difluoro-4-bora-3a,4a-diaza-s-indacene) such as BODIPY™ 500/510 (e.g. 12-(2,2-difluoro-12-methyl-1-aza-3-azonia-2-boranuidatricyclo[7.3.0.03,7]dodeca-3,5,7,9,11-pentaen-4-yl)dodecanoic acid), and derivatives thereof,
naphthalene-based dyes, in particular prodan (6-propionyl-2-dimethyaminonaphthalene) and derivatives thereof,
pyrene-based dyes, in particular Alexa Fluor™ 405 (e.g. 8-[2-[4-(2,5-dioxopyrrolidin-1-yl)oxycarbonylpiperidin-1-yl]-2-oxoethoxy]pyrene-1,3,6-trisulfonate; triethylazanium), Cascade Blue™ (e.g. [4-[[4-(diethylamino)phenyl]-[4-(ethylamino)naphthalen-2-yl]methylidene]cyclohexa-2,5-dien-1-ylidene]-diethylazanium) and derivatives thereof,
squaraine-based dyes, in particular Seta dyes such as Seta 650 (CAS registry number 2043938-22-1), Seta 375 (CAS registry number 2043938-18-5) and derivatives thereof,
squaraine rotaxane-based dyes, in particular SeTau dyes such as SeTau 647 (such as SeTau 647 Di-NHS, CAS registry number 1442086-02-3), SeTau 488 (such as SeTau-488-NHS, Seta Biomedicals, product number K9-3152, Molecular Mass: 1642.73, λ_{Ab} max. [nm]:486; ε [M⁻¹ cm⁻¹]:59000; λ_{Em} max. [nm]:532; QY [%]:27) and derivatives thereof,
xanthene-based dyes, in particular rhodamine dyes such as MB™ 660R (e.g. MB™ 660R acid, see FIG. 15), ROX (Carboxy-X-rhodamine), TAMRA (carboxytetramethylrhodamine), TEX™ 615 (CAS registry number 1209486-62-3) and derivatives thereof, fluorescein dyes such as Alexa Fluor™ 488 (e.g. Alexa Fluor™ 488 acid, see FIG. 15), 6-FAM (6-Carboxyfluorescein), HEX (Hexachlorofluorescein), JOE (6-carboxy-4′,5′-dichloro-2′,7′-dimethoxyfluorescein), MAX (CAS registry number 1610888-25-9), TET (Tetrachlorofluorescein) and SUN (e.g. see FIG. 15) and derivatives thereof, and carbopyronine-based dyes such as ATTO™ 647 (e.g. ATTO™ 647 acid see FIG. 15) and derivatives thereof,
and 4′,6-diamidino-2-phenylindole (DAPI),
and combinations thereof.

More particularly, said at least one fluorescent dye may be selected from the group consisting of
anthracene-based dyes, in particular DRAQ5™, CyTRAK Orange™ and derivatives thereof,
coumarin-based dyes, in particular Alexa Fluor™ 350, Pacific Blue™ and derivatives thereof,
Cyanine-based dyes, in particular Cy5™ or Cy5.5™, including sulfonated and non-sulfonated forms, preferably sulfonated forms such as sulfonated Cy5™ or sulfonated Cy5.5™, indocyanine green and derivatives thereof,
dipyrromethene-based dyes, in particular BODIPY™ dyes such as BODIPY™ 500/510, and derivatives thereof,
naphthalene-based dyes, in particular prodan and derivatives thereof,
pyrene-based dyes, in particular Alexa Fluor™ 405, Cascade Blue™ and derivatives thereof,
squaraine-based dyes, in particular Seta dyes such as Seta 650, Seta 375 and derivatives thereof,
squaraine rotaxane-based dyes, in particular SeTau dyes such as SeTau 647, SeTau 488 and derivatives thereof,
xanthene-based dyes, in particular rhodamine dyes such as MB™ 660R, ROX, TAMRA, TEX™ 615 and derivatives thereof, fluorescein dyes such as Alexa Fluor™ 488, 6-FAM, HEX, JOE, MAX, TET and SUN and derivatives thereof, and carbopyronine-based dyes such as ATTO™ 647 and derivatives thereof,
and 4′,6-diamidino-2-phenylindole (DAPI),
and combinations thereof.

More particularly, said at least one fluorescent dye may be selected from the group consisting of anthracene-based dyes, coumarin-based dyes, cyanine-based dyes, dipyrromethene-based dyes, naphthalene-based dyes, pyrene-based dyes, squaraine-based dyes, squaraine rotaxane-based dyes, xanthene-based dyes and 4′,6-diamidino-2-phenylindole (DAPI), and combinations thereof. Even more particularly, said at least one fluorescent dye may be selected from the group consisting of anthracene-based dyes, coumarin-based dyes, cyanine-based dyes, dipyrromethene-based dyes, naphthalene-based dyes, pyrene-based dyes, squaraine-based dyes, squaraine rotaxane-based dyes and xanthene-based dyes, and combinations thereof.

In a particular embodiment, said at least one fluorescent dye may be selected from the group consisting of Acridine orange, DRAQ5™, Cytrak Orange, Alexa Fluor™ 350, Pacific Blue, Cy5™, preferably Sulfo-Cy5™, CY5.5, preferably Sulfo-Cy5.5, BODIPY™ 500/510, prodan, Alexa Fluor™ 405, Cascade Blue, Seta™ 650, Seta™ 375, Setau™ 647, SeTau 488, MB™ 660R, Alexa Fluor™ 488, ATTO™ 647 and DAPI, derivatives thereof, and combinations thereof.

In another particular embodiment, said at least one fluorescent dye may be selected from the group consisting of DRAQ5™, Alexa Fluor™ 350, Pacific Blue, Cy5™, preferably Sulfo-Cy5™, BODIPY™ 500/510, prodan, Alexa Fluor™ 405, Cascade Blue, Seta™ 650, Setau™ 647, SeTau 488, MB™ 660R, Alexa Fluor™ 488, ATTO™ 647 and DAPI, derivatives thereof, and combinations thereof.

In another particular embodiment, said at least one fluorescent dye may be selected from the group consisting of DRAQ5™, Alexa Fluor™ 350, Cy5™, preferably Sulfo-Cy5™, BODIPY™ 500/510, prodan, Alexa Fluor™ 405, Seta™ 650, Setau™ 647, MB™ 660R, Alexa Fluor™ 488, ATTO™ 647 and DAPI, derivatives thereof, and combinations thereof.

In another particular embodiment, said at least one fluorescent dye may be selected from the group consisting of DRAQ5™, Cytrak Orange, Alexa Fluor™ 350, Pacific Blue, Cy5™, preferably Sulfo-Cy5™, CY5.5, preferably Sulfo-Cy5.5, BODIPY™ 500/510, prodan, Alexa Fluor™ 405, Cascade Blue, Seta™ 650, Seta™ 375, Setau™ 647, SeTau 488, MB™ 660R, Alexa Fluor™ 488, ATTO™ 647 and DAPI, derivatives thereof, and combinations thereof.

In another particular embodiment, said at least one fluorescent dye may be selected from the group consisting of Acridine orange, DRAQ5™, Cytrak Orange, Alexa Fluor™ 350, Pacific Blue, Cy5™, preferably Sulfo-Cy5™, CY5.5, preferably Sulfo-Cy5.5, BODIPY™ 500/510, prodan, Alexa Fluor™ 405, Cascade Blue, Seta™ 650, Seta™ 375, Setau™ 647, SeTau 488, MB™ 660R, Alexa Fluor™ 488, and ATTO™ 647, derivatives thereof, and combinations thereof.

In another particular embodiment, said at least one fluorescent dye may be selected from the group consisting of DRAQ5™, Cytrak Orange, Alexa Fluor™ 350, Pacific Blue, Cy5™, preferably Sulfo-Cy5™, CY5.5, preferably Sulfo-Cy5.5, BODIPY™ 500/510, prodan, Alexa Fluor™ 405, Cascade Blue, Seta™ 650, Seta™ 375, Setau™ 647, SeTau 488, MB™ 660R, Alexa Fluor™ 488, and ATTO™ 647, derivatives thereof, and combinations thereof.

As used herein, the term “derivative” refers to a fluorescent compound comprising the core structure of said parent compound (i.e. the aromatic ring skeleton that defines the dye's color or absorption/emission wavelengths, e.g. the core structure of anthracene, coumarin, cyanine, dipyrromethene, naphthalene, pyrene, squaraine, squaraine rotaxane, xanthene, rhodamine, fluorescein or carbopyronine dyes) and that differs from said parent compound in virtue of one or several chemical modifications, e.g. in virtue of modifications in substituents. For example, the derivative may differ from the parent compound by the replacement of at least one substituent by another substituent, by the deletion of at least one substituent, by the addition of at least one substituent and combinations thereof. For example, a hydrogen may be replaced by a halogen, such as fluorine or chlorine, a hydroxyl group (—OH) may be replaced by a carboxylic acid moiety (—COOH), or a carboxylic acid moiety may be substituted with a hydrogen. Any kind of substituent modifications can be contemplated with proviso that the resulting derivative is a fluorophore and suitable for implementing the method of the invention.

Preferably, derivatives of a fluorescent dye are selected from the group consisting of ester derivatives, in particular NHS ester (succinimidyl ester) derivatives, TFP ester (tetrafluorophenyl ester) derivatives, STP (4-Sulfo-2,3,5,6-Tetrafluorophenol, Sodium Salt) ester derivatives or sulfo-NHS ester derivatives, carboxylic acid derivatives, dibenzocyclooctyne (DBCO) derivatives, tetrazine derivatives, hydrazide derivatives, azide derivatives, trans-cyclooctene (TCO) derivatives, alkyne derivatives, maleimide derivatives. More preferably, derivatives of a fluorescent dye are selected from the group consisting of carboxylic acid derivatives, dibenzocyclooctyne (DBCO) derivatives, NHS ester derivatives and maleimide derivatives.

In a more particular embodiment, said at least one fluorescent dye is selected from the group consisting of DRAQ5™, Alexa Fluor™ 350 NHS ester, Pacific Blue-NHS Ester, Sulfo-Cy5 acid, BODIPY™ 500/510, prodan, Alexa Fluor™ 405-DBCO, Cascade Blue, Seta™ 650-DBCO, SeTau 488-NHS, SeTau™ 647-maleimide, MB™ 660R-acid, MB™ 660R-DBCO, Alexa Fluor™ 488-acid, Alexa Fluor™ 488-DBCO, ATTO™ 647-acid and DAPI, and combinations thereof.

In another more particular embodiment, said at least one fluorescent dye is selected from the group consisting of DRAQ5™, Alexa Fluor™ 350 NHS ester, Sulfo-Cy5 acid, BODIPY™ 500/510, prodan, Alexa Fluor™ 405-DBCO, Seta™ 650-DBCO, SeTau™ 647-maleimide, MB™ 660R-acid, MB™ 660R-DBCO, Alexa Fluor™ 488-acid, Alexa Fluor™ 488-DBCO, ATTO™ 647-acid and DAPI, and combinations thereof.

In another more particular embodiment, said at least one fluorescent dye may be selected from the group consisting of Acridine orange, DRAQ5™, Cytrak Orange, Alexa Fluor™ 350 NHS ester, Pacific Blue-NHS Ester, Sulfo-Cy5 acid, Sulfo-Cy5.5 acid, preferably Sulfo-Cy5.5, BODIPY™ 500/510, prodan, Alexa Fluor™ 405-DBCO, Cascade Blue, Seta™ 650-DBCO, Seta 375-NHS, SeTau™ 647-maleimide, SeTau 488-NHS, MB™ 660R-acid, MB™ 660R-DBCO, Alexa Fluor™ 488-acid, Alexa Fluor™ 488-DBCO, ATTO™ 647-acid and DAPI, derivatives thereof, and combinations thereof.

In another more particular embodiment, said at least one fluorescent dye may be selected from the group consisting of DRAQ5™, Cytrak Orange, Alexa Fluor™ 350 NHS ester, Pacific Blue-NHS Ester, Sulfo-Cy5 acid, Sulfo-Cy5.5 acid, preferably Sulfo-Cy5.5, BODIPY™ 500/510, prodan, Alexa Fluor™ 405-DBCO, Cascade Blue, Seta™ 650-DBCO, Seta 375-NHS, SeTau™ 647-maleimide, SeTau 488-NHS, MB™ 660R-acid, MB™ 660R-DBCO, Alexa Fluor™ 488-acid, Alexa Fluor™ 488-DBCO, ATTO™ 647-acid and DAPI, derivatives thereof, and combinations thereof.

In another more particular embodiment, said at least one fluorescent dye may be selected from the group consisting of Acridine orange, DRAQ5™, Cytrak Orange, Alexa Fluor™ 350 NHS ester, Pacific Blue-NHS Ester, Sulfo-Cy5 acid, Sulfo-Cy5.5 acid, preferably Sulfo-Cy5.5, BODIPY™ 500/510, prodan, Alexa Fluor™ 405-DBCO, Cascade Blue, Seta™ 650-DBCO, Seta 375-NHS, SeTau™ 647-maleimide, SeTau 488-NHS, MB™ 660R-acid, MB™ 660R-DBCO, Alexa Fluor™ 488-acid, Alexa Fluor™ 488-DBCO and ATTO™ 647-acid, derivatives thereof, and combinations thereof.

In another more particular embodiment, said at least one fluorescent dye may be selected from the group consisting of DRAQ5™, Cytrak Orange, Alexa Fluor™ 350 NHS ester, Pacific Blue-NHS Ester, Sulfo-Cy5 acid, Sulfo-Cy5.5 acid, preferably Sulfo-Cy5.5, BODIPY™ 500/510, prodan, Alexa Fluor™ 405-DBCO, Cascade Blue, Seta™ 650-DBCO, Seta 375-NHS, SeTau™ 647-maleimide, SeTau 488-NHS, MB™ 660R-acid, MB™ 660R-DBCO, Alexa Fluor™ 488-acid, Alexa Fluor™ 488-DBCO and ATTO™ 647-acid, derivatives thereof, and combinations thereof.

Preferably, in the method of the invention, the sample is contacted with 1, 2, 3 or 4 fluorescent dyes, more preferably with 1 or 2 fluorescent dyes.

In some embodiments, said at least one fluorescent dye is a combination selected from the group consisting of Acridine orange—DAPI, Acridine orange—Seta 375-NHS, Acridine orange—MB660R acid, Acridine orange—Alexa Fluor 350-NHS Ester, Acridine orange—Pacific Blue-NHS Ester, Acridine orange—Sulfo Cy5-acid, Acridine orange—BODIPY, Acridine orange—Prodan, Acridine orange—Alexa Fluor 405-DBCO, Acridine orange—Cascade blue, Acridine orange—Alexa Fluor 488-Acid, DRAQ5—Alexa Fluor 350-NHS Ester, DRAQ5—Pacific Blue-NHS Ester, DRAQ5—Sulfo Cy5-acid, DRAQ5—BODIPY 500/510, DRAQ5—Prodan, DRAQ5—Alexa Fluor 405-DBCO, DRAQ5—Cascade blue, DRAQ5—Seta 650-DBCO, DRAQ5—SeTau 647-Maleimide, DRAQ5—MB660R-Acid, DRAQ5—MB660R-DBCO, DRAQ5—ATTO647-Acid, Cytrak orange—Alexa Fluor 350-NHS Ester, Cytrak orange—Pacific Blue-NHS Ester, Cytrak orange—Sulfo Cy5-acid, Cytrak orange—BODIPY 500/510, Cytrak orange—Prodan, Cytrak orange—Alexa Fluor 405-DBCO, Cytrak orange—Cascade blue, Seta 375-NHS—Alexa Fluor 350-NHS Ester, Seta 375-NHS—Pacific Blue-NHS Ester, Seta 375-NHS—Sulfo Cy5-acid, Seta 375-NHS—BODIPY 500/510, Seta 375-NHS—Prodan, Seta 375-NHS—Alexa Fluor 405-DBCO, Seta 375-NHS—Cascade blue, Alexa Fluor 488-Acid—Alexa Fluor 350-NHS Ester, Alexa Fluor 488-Acid—Pacific Blue-NHS Ester, Alexa Fluor 488-Acid—Sulfo Cy5-acid, Alexa Fluor 488-Acid—BODIPY 500/510, Alexa Fluor 488-Acid—Prodan, Alexa Fluor 488-Acid—Alexa Fluor 405-DBCO, Alexa Fluor 488-Acid—Cascade blue, MB660R-Acid—Alexa Fluor 350-NHS Ester, MB660R-Acid—Pacific Blue-NHS Ester, MB660R-Acid—Sulfo Cy5-acid, MB660R-Acid—BODIPY 500/510, MB660R-Acid—Prodan, MB660R-Acid—Alexa Fluor 405-DBCO, MB660R-Acid—Cascade blue, MB660R-Acid—Seta 650-DBCO, MB660R-Acid—SeTau 647-Maleimide, MB660R-DBCO—Alexa Fluor 350-NHS Ester, MB660R-DBCO—Pacific Blue-NHS Ester, MB660R-DBCO—Sulfo Cy5-acid, MB660R-DBCO—BODIPY 500/510, MB660R-DBCO—Prodan, MB660R-DBCO—Alexa Fluor 405-DBCO, MB660R-DBCO—Cascade blue, MB660R-DBCO—Seta 650-DBCO, MB660R-DBCO—SeTau 647-Maleimide, ATTO647-Acid—Sulfo Cy5-acid, ATTO647-Acid—Seta 650-DBCO, ATTO647-Acid—SeTau 647-Maleimide, ATTO647-Acid—MB660R-Acid, ATTO647-Acid—MB660R-DBCO, Sulfo Cy5.5-acid—Sulfo Cy5-acid, Sulfo Cy5-acid—Seta 650-DBCO, Sulfo Cy5-acid—SeTau 647-Maleimide and Seta 650-DBCO—SeTau 647-Maleimide, preferably selected from the group consisting of Acridine orange—Seta 375-NHS, Acridine orange—MB660R acid, Acridine orange—Alexa Fluor 350-NHS Ester, Acridine orange—Pacific Blue-NHS Ester, Acridine orange—Sulfo Cy5-acid, Acridine orange—BODIPY, Acridine orange—Prodan, Acridine orange—Alexa Fluor 405-DBCO, Acridine orange—Cascade blue, Acridine orange—Alexa Fluor 488-Acid, DRAQ5—Alexa Fluor 350-NHS Ester, DRAQ5—Pacific Blue-NHS Ester, DRAQ5—Sulfo Cy5-acid, DRAQ5—BODIPY 500/510, DRAQ5—Prodan, DRAQ5—Alexa Fluor 405-DBCO, DRAQ5—Cascade blue, DRAQ5—Seta 650-DBCO, DRAQ5—SeTau 647-Maleimide, DRAQ5—MB660R-Acid, DRAQ5—MB660R-DBCO, DRAQ5—ATTO647-Acid, Cytrak orange—Alexa Fluor 350-NHS Ester, Cytrak orange—Pacific Blue-NHS Ester, Cytrak orange—Sulfo Cy5-acid, Cytrak orange—BODIPY 500/510, Cytrak orange—Prodan, Cytrak orange—Alexa Fluor 405-DBCO, Cytrak orange—Cascade blue, Seta 375-NHS—Alexa Fluor 350-NHS Ester, Seta 375-NHS—Pacific Blue-NHS Ester, Seta 375-NHS—Sulfo Cy5-acid, Seta 375-NHS—BODIPY 500/510, Seta 375-NHS—Prodan, Seta 375-NHS—Alexa Fluor 405-DBCO, Seta 375-NHS—Cascade blue, Alexa Fluor 488-Acid—Alexa Fluor 350-NHS Ester, Alexa Fluor 488-Acid—Pacific Blue-NHS Ester, Alexa Fluor 488-Acid—Sulfo Cy5-acid, Alexa Fluor 488-Acid—BODIPY 500/510, Alexa Fluor 488-Acid—Prodan, Alexa Fluor 488-Acid—Alexa Fluor 405-DBCO, Alexa Fluor 488-Acid—Cascade blue, MB660R-Acid—Alexa Fluor 350-NHS Ester, MB660R-Acid—Pacific Blue-NHS Ester, MB660R-Acid—Sulfo Cy5-acid, MB660R-Acid—BODIPY 500/510, MB660R-Acid—Prodan, MB660R-Acid—Alexa Fluor 405-DBCO, MB660R-Acid—Cascade blue, MB660R-Acid—Seta 650-DBCO, MB660R-Acid—SeTau 647-Maleimide, MB660R-DBCO—Alexa Fluor 350-NHS Ester, MB660R-DBCO—Pacific Blue-NHS Ester, MB660R-DBCO—Sulfo Cy5-acid, MB660R-DBCO—BODIPY 500/510, MB660R-DBCO—Prodan, MB660R-DBCO—Alexa Fluor 405-DBCO, MB660R-DBCO—Cascade blue, MB660R-DBCO—Seta 650-DBCO, MB660R-DBCO—SeTau 647-Maleimide, ATTO647-Acid—Sulfo Cy5-acid, ATTO647-Acid—Seta 650-DBCO, ATTO647-Acid—SeTau 647-Maleimide, ATTO647-Acid—MB660R-Acid, ATTO647-Acid—MB660R-DBCO, Sulfo Cy5.5-acid—Sulfo Cy5-acid, Sulfo Cy5-acid—Seta 650-DBCO, Sulfo Cy5-acid—SeTau 647-Maleimide and Seta 650-DBCO—SeTau 647-Maleimide.

In a particular embodiment, the microorganism or group of microorganisms of interest belongs to or comprises a microorganism belonging to the group of thermophilic acidophilic bacteria (TAB) as defined above, i.e. belonging to the Alicyclobacillus genus, preferably selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius and Alicyclobacillus contaminans, and combinations thereof, and said at least one fluorescent dye is selected from the group consisting of xanthene-based dyes, preferably selected from the group consisting of rhodamine dyes, fluorescein dyes and carbopyronine-based dyes, and combinations thereof, more preferably selected from the group consisting of Alexa Fluor 488, MB660R and ATTO647, and derivatives and combinations thereof, even more preferably selected from the group consisting of Alexa Fluor 488-acid, Alexa Fluor 488-DBCO, MB660R-acid, MB660R-DBCO and ATTO647-acid, and combinations thereof. In a particularly preferred embodiment, the microorganism or group of microorganisms of interest belongs to or comprises a microorganism belonging to the group of thermophilic acidophilic bacteria (TAB) as defined above, i.e. belonging to the Alicyclobacillus genus, preferably selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius and Alicyclobacillus contaminans, and combinations thereof, and said at least one fluorescent dye is selected from the group consisting of MB660R, and derivatives thereof, preferably selected from the group consisting of MB660R-acid and MB660R-DBCO, and combinations thereof, more preferably is MB660R-DBCO.

In another particular embodiment, the microorganism or group of microorganisms of interest belongs to or comprises a microorganism belonging to the group of acetic acid bacteria (AAB) as defined above, preferably selected from bacteria belonging to the genera Acetobacter, Acidomonas, Asaia, Gluconacetobacter, Gluconobacter, Ameyamaea, Bombella, Commensalibacter, Endobacter, Granulibacter, Komagataeibacter, Kozakia, Neoasaia, Neokomagataea, Nguyenibacter, Saccharibacter, Swaminathania, Swingsia and Tanticharoenia, and combinations thereof, more preferably selected from bacteria belonging to the genera Acetobacter, Gluconobacter, Gluconacetobacter and Asaia, and combinations thereof, and even more preferably selected from the group consisting of Acetobacter aceti, Gluconoacetobacter liquefaciens, Gluconobacter oxydans and Asaia siamensis, and combinations thereof, and said at least one fluorescent dye is selected from the group consisting of xanthene-based dyes, preferably selected from the group consisting of rhodamine dyes, fluorescein dyes, carbopyronine-based dyes and DAPI, and combinations thereof, more preferably selected from the group consisting of Alexa Fluor 488, MB660R, ATTO647 and DAPI, and derivatives and combinations thereof, even more preferably selected from the group consisting of Alexa Fluor 488-acid, Alexa Fluor 488-DBCO, MB660R-acid, MB660R-DBCO, ATTO647-acid and DAPI, and combinations thereof. Preferably, said at least one fluorescent dye is selected from the group consisting of Alexa Fluor 488, MB660R and ATTO647, and derivatives and combinations thereof, more preferably selected from the group consisting of Alexa Fluor 488-acid, Alexa Fluor 488-DBCO, MB660R-acid, MB660R-DBCO and ATTO647-acid, and combinations thereof.

In a particularly preferred embodiment, the microorganism or group of microorganisms of interest belongs to or comprises a microorganism belonging to the group of acetic acid bacteria (AAB) as defined above, preferably selected from bacteria belonging to the genera Acetobacter, Gluconobacter, Gluconacetobacter and Asaia, and combinations thereof, and even more preferably selected from the group consisting of Acetobacter aceti, Gluconoacetobacter liquefaciens, Gluconobacter oxydans and Asaia siamensis, and combinations thereof, and said at least one fluorescent dye is selected from the group consisting of Alexa Fluor 488, MB660R and ATTO647, and derivatives and combinations thereof, preferably selected from the group consisting of Alexa Fluor 488-acid, MB660R-acid and ATTO647-acid, and combinations thereof, more preferably selected from the group consisting of MB660R and ATTO647, and derivatives and combinations thereof, in particular selected from the group consisting of MB660R-acid and ATTO647-acid, and combination thereof.

In another particular embodiment, the microorganism or group of microorganisms of interest belongs to or comprises a microorganism belonging to the group of lactic acid bacteria (LAB) as defined above, preferably selected from bacteria belonging to the genera Lactobacillus, Leuconostoc, Pediococcus, Lactococcus, Enterococcus, Carnobacterium, Streptococcus and Weissella, and combinations thereof, more preferably selected from bacteria belonging to the genera Lactobacillus and Weissella, and combinations thereof, and even more preferably selected from the group consisting of Lactobacillus casei, Lactobacillus plantarum and Weissella confusa, and combinations thereof, and said at least one fluorescent dye is selected from the group consisting of xanthene-based dyes, preferably selected from the group consisting of rhodamine dyes, fluorescein dyes, carbopyronine-based dyes and DAPI, and combinations thereof, more preferably selected from the group consisting of Alexa Fluor 488, MB660R, ATTO647, and DAPI, and derivatives and combinations thereof, even more preferably selected from the group consisting of Alexa Fluor 488-acid, Alexa Fluor 488-DBCO, MB660R-acid, MB660R-DBCO and ATTO647-acid and combinations thereof. In a particularly preferred embodiment, the microorganism or group of microorganisms of interest belongs to or comprises a microorganism belonging to the group of lactic acid bacteria (LAB) as defined above, preferably selected from bacteria belonging to the genera Lactobacillus and Weissella, and combinations thereof, and even more preferably selected from the group consisting of Lactobacillus casei, Lactobacillus plantarum and Weissella confusa, and combinations thereof, and said at least one fluorescent dye is selected from the group consisting of Alexa Fluor 488, MB660R, ATTO647, and DAPI, and derivatives and combinations thereof, preferably selected from the group consisting of Alexa Fluor 488-acid, Alexa Fluor 488-DBCO, MB660R-acid, MB660R-DBCO, ATTO647-acid and DAPI, and combinations thereof. In preferred embodiments, said at least one fluorescent dye is selected from the group consisting of Alexa Fluor 488, MB660R and DAPI, and derivatives and combinations thereof, preferably selected from the group consisting of Alexa Fluor 488-acid, MB660R-acid and DAPI, and combinations thereof, more preferably selected from the group consisting of Alexa Fluor 488-acid and MB660R-acid and combinations thereof, even more preferably is MB660R-acid. In another preferred embodiment, said at least one fluorescent dye is selected from the group consisting of Alexa Fluor 488 and MB660R, and derivatives and combinations thereof, preferably selected from the group consisting of Alexa Fluor 488-acid and MB660R-acid, and combinations thereof.

In another particular embodiment, the microorganism or group of microorganisms of interest belongs to or comprises a microorganism belonging to the group of anaerobic bacteria, preferably selected from Clostridium and Cutibacterium bacteria, and combinations thereof, more preferably, selected from the group consisting of Clostridium sporogenes and Cutibacterium acnes, and combinations thereof, and said at least one fluorescent dye is selected from the group consisting of xanthene-based dyes, preferably rhodamine dyes, cyanine-based dyes and DAPI, and combinations thereof, preferably selected from the group consisting of MB660R, Cy5™, preferably sulfo-Cy5, and DAPI, and derivatives and combinations thereof, more preferably from the group consisting of MB660R-DBCO, Sulfo-Cy5 acid and DAPI, and combinations thereof. In particular, in this embodiment, said at least one fluorescent dye may be a combination of MB660R and Cy5™, preferably sulfo-Cy5, in particular a combination of MB660R-DBCO and Sulfo-Cy5 acid.

In another particular embodiment, the microorganism or group of microorganisms of interest belongs to or comprises a microorganism belonging to the group of aerobic mesophilic bacteria (AMB) as defined above, preferably selected from bacteria belonging to the genera Methylobacterium, Pseudomonas, Bacillus, Escherichia, Staphylococcus, Acinetobacter, Cronobacter, Klebsiella, Salmonella, Enterococcus, Listeria, Shigella, Kocuria and Burkholderia, and combinations thereof, more preferably selected from the group consisting of Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Listeria grayi, Shigella sonnei, Kocuria rhizophila and Burkholderia cepacia, and combinations thereof, and said at least one fluorescent dye is selected from the group consisting of xanthene-based dyes, preferably selected from the group consisting of rhodamine dyes, fluorescein dyes and carbopyronine-based dyes, anthracene-based dyes, coumarin-based dyes, cyanine-based dyes, dipyrromethene-based dyes, naphthalene-based dyes, pyrene-based dyes, squaraine-based dyes, squaraine rotaxane-based dyes and DAPI, and combinations thereof, preferably selected from the group consisting of DRAQ5™, Alexa Fluor™ 350, Pacific Blue, Cy5™, preferably Sulfo-Cy5, BODIPY™ dyes, preferably BODIPY 500/510, prodan, Alexa Fluor™ 405, Cascade Blue, Seta dyes, preferably Seta 650, SeTau dyes, preferably SeTau 647 or SeTau 488, Alexa Fluor 488, MB660R, ATTO647 and DAPI, and derivatives and combinations thereof, more preferably selected from the group consisting of DRAQ5™, Alexa Fluor™ 350-NHS ester, Pacific Blue-NHS Ester, Sulfo-Cy5-acid, BODIPY 500/510, prodan, Alexa Fluor™ 405-DBCO, Cascade Blue, Seta 650-DBCO, SeTau 647-Maleimide, SeTau 488-NHS, Alexa Fluor 488-acid, Alexa Fluor 488-DBCO, MB660R-acid, MB660R-DBCO, ATTO647-acid and DAPI, and combinations thereof. In particular, said at least one fluorescent dye may be selected from the group consisting of DRAQ5™, Alexa Fluor™ 350, Cy5™, preferably Sulfo-Cy5, BODIPY™ dyes, preferably BODIPY 500/510, prodan, Alexa Fluor™ 405, Seta dyes, preferably Seta 650, SeTau dyes, preferably SeTau 647, Alexa Fluor 488, MB660R, ATTO647 and DAPI, and derivatives and combinations thereof, more preferably selected from the group consisting of DRAQ5™, Alexa Fluor™ 350-NHS ester, Sulfo-Cy5-acid, BODIPY 500/510, prodan, Alexa Fluor™ 405-DBCO, Seta 650-DBCO, SeTau 647-Maleimide, Alexa Fluor 488-acid, Alexa Fluor 488-DBCO, MB660R-acid, MB660R-DBCO, ATTO647-acid and DAPI, and combinations thereof.

More particularly, the microorganism or group of microorganisms of interest may belong to or comprise a microorganism belonging to the group of aerobic mesophilic bacteria (AMB) as defined above, preferably selected from bacteria belonging to the genera Methylobacterium, Pseudomonas, Bacillus, Escherichia, Staphylococcus, Acinetobacter, Cronobacter, Klebsiella, Salmonella, Enterococcus, Shigella, Kocuria and Burkholderia, and combinations thereof, more preferably selected from the group consisting of Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Shigella sonnei, Kocuria rhizophila and Burkholderia cepacia, and combinations thereof, and said at least one fluorescent dye is selected from the group consisting of acridine dyes, xanthene-based dyes, preferably selected from the group consisting of rhodamine dyes, fluorescein dyes and carbopyronine-based dyes, anthracene-based dyes, coumarin-based dyes, cyanine-based dyes, dipyrromethene-based dyes, naphthalene-based dyes, pyrene-based dyes, squaraine-based dyes, squaraine rotaxane-based dyes and DAPI, and combinations thereof, preferably selected from the group consisting of acridine orange, DRAQ5™, Cytrak orange, Alexa Fluor™ 350, Pacific Blue, Cy5™, preferably Sulfo-Cy5, Cy5.5™, preferably Sulfo-Cy5.5, BODIPY™ dyes, preferably BODIPY 500/510, prodan, Alexa Fluor™ 405, Cascade Blue, Seta dyes, preferably Seta 650 or Seta 375, SeTau dyes, preferably SeTau 647 or SeTau 488, Alexa Fluor 488, MB660R, ATTO647 and DAPI, and derivatives and combinations thereof, more preferably selected from the group consisting of acridine orange, DRAQ5™, Cytrak orange, Alexa Fluor™ 350-NHS ester, Pacific Blue-NHS Ester, Sulfo-Cy5-acid, Sulfo-Cy5.5-acid, BODIPY 500/510, prodan, Alexa Fluor™ 405-DBCO, Cascade Blue, Seta 650-DBCO, Seta 375-NHS, SeTau 647-Maleimide, SeTau 488-NHS, Alexa Fluor 488-acid, Alexa Fluor 488-DBCO, MB660R-acid, MB660R-DBCO, ATTO647-acid and DAPI, and combinations thereof. In particular, said at least one fluorescent dye may be selected from the group consisting of DRAQ5™, Cytrak orange, Alexa Fluor™ 350, Pacific Blue, Cy5™, preferably Sulfo-Cy5, Cy5.5™, preferably Sulfo-Cy5.5, BODIPY™ dyes, preferably BODIPY 500/510, prodan, Alexa Fluor™ 405, Cascade Blue, Seta dyes, preferably Seta 650 or Seta 375, SeTau dyes, preferably SeTau 647 or SeTau 488, Alexa Fluor 488, MB660R and ATTO647, and derivatives and combinations thereof, more preferably selected from the group consisting of DRAQ5™, Cytrak orange, Alexa Fluor™ 350-NHS ester, Pacific Blue-NHS Ester, Sulfo-Cy5-acid, Sulfo-Cy5.5-acid, BODIPY 500/510, prodan, Alexa Fluor™ 405-DBCO, Cascade Blue, Seta 650-DBCO, Seta 375-NHS, SeTau 647-Maleimide, SeTau 488-NHS, Alexa Fluor 488-acid, Alexa Fluor 488-DBCO, MB660R-acid, MB660R-DBCO and ATTO647-acid, and combinations thereof.

In a preferred embodiment, the microorganism or group of microorganisms of interest may belong to or comprise a microorganism belonging to the group of aerobic mesophilic bacteria (AMB) as defined above, preferably selected from bacteria belonging to the genera Methylobacterium, Pseudomonas, Bacillus, Escherichia, Staphylococcus, Acinetobacter, Cronobacter, Klebsiella, Salmonella, Enterococcus, Shigella, Kocuria and Burkholderia, and combinations thereof, more preferably selected from the group consisting of Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Shigella sonnei, Kocuria rhizophila and Burkholderia cepacia, and combinations thereof, and said at least one fluorescent dye is selected from the group consisting of MB660R, and derivatives and combinations thereof, optionally in combination with a fluorescent dye selected from the group consisting of Cy5™, preferably Sulfo-Cy5. Preferably, said at least one fluorescent dye is MB660R-DBCO, optionally in combination with Sulfo-Cy5-Acid.

In another particular embodiment, the microorganism or group of microorganisms of interest belongs to or comprises a microorganism belonging to the group of Gram negative bacteria as defined above, preferably selected from bacteria belonging to the genera Escherichia and Pseudomonas, and combinations thereof, more preferably selected from Escherichia coli and Pseudomonas aeruginosa, and combinations thereof, and said at least one fluorescent dye is selected from the group consisting of xanthene-based dyes, preferably rhodamine dyes, pyrene-based dyes, coumarin-based dyes, cyanine-based dyes and DAPI, and combinations thereof, preferably selected from the group consisting of Cy5™, preferably Sulfo-Cy5, Alexa Fluor™ 350, Alexa Fluor™ 405, MB660R and DAPI, and derivatives and combinations thereof, more preferably selected from the group consisting of Sulfo-Cy5-acid, Alexa Fluor™ 350-NHS ester, Alexa Fluor™ 405-DBCO, MB660R-DBCO and DAPI, and combinations thereof. Even more preferably, said at least one fluorescent dye is selected from the group consisting of Cy5™, preferably Sulfo-Cy5, Alexa Fluor™ 350, Alexa Fluor™ 405 and MB660R, and derivatives and combinations thereof, more preferably selected from the group consisting of Sulfo-Cy5-acid, Alexa Fluor™ 350-NHS ester, Alexa Fluor™ 405-DBCO and MB660R-DBCO, and combinations thereof.

More particularly, said at least one fluorescent dye may be selected from the group consisting of xanthene-based dyes, preferably rhodamine dyes, and cyanine-based dyes, and combinations thereof, preferably selected from the group consisting of Cy5™, preferably Sulfo-Cy5, MB660R, and derivatives and combinations thereof, more preferably selected from the group consisting of Sulfo-Cy5-acid and MB660R-DBCO, and combinations thereof. Alternatively, said at least one fluorescent dye may be selected from the group consisting of pyrene-based dyes, coumarin-based dyes, and combinations thereof, preferably selected from the group consisting of Alexa Fluor™ 350 and Alexa Fluor™ 405, and derivatives and combinations thereof, more preferably selected from the group consisting of Alexa Fluor™ 350-NHS ester, Alexa Fluor™ 405-DBCO, and combinations thereof. In particular, said at least one fluorescent dye may be a combination of Alexa Fluor™ 350-NHS ester, Alexa Fluor™ 405-DBCO.

In another particular embodiment, the microorganism or group of microorganisms of interest belongs to or comprises a microorganism belonging to the group of Gram negative bacteria as defined above, preferably selected from bacteria belonging to the genera Escherichia, Proteus and Pseudomonas, and combinations thereof, more preferably selected from Escherichia coli, Proteus mirabilis and Pseudomonas aeruginosa, and combinations thereof, and said at least one fluorescent dye may be selected from the group consisting of xanthene-based dyes, preferably rhodamine dyes, cyanine-based dyes and DAPI and combinations thereof, preferably selected from the group consisting of Cy5™, preferably Sulfo-Cy5, MB660R and DAPI, and derivatives and combinations thereof, more preferably selected from the group consisting of Sulfo-Cy5-acid, MB660R-DBCO and DAPI, and combinations thereof. More particularly, said at least one fluorescent dye may be selected from the group consisting of xanthene-based dyes, preferably rhodamine dyes, and cyanine-based dyes, and combinations thereof, preferably selected from the group consisting of Cy5™, preferably Sulfo-Cy5, MB660R, and derivatives and combinations thereof, more preferably selected from the group consisting of Sulfo-Cy5-acid and MB660R-DBCO, and combinations thereof.

In another particular embodiment, the microorganism or group of microorganisms of interest belongs to or comprises a microorganism belonging to the group of Gram positive bacteria as defined above, preferably selected from bacteria belonging to the genera Staphylococcus and Enterococcus, and combinations thereof, more preferably selected from Staphylococcus aureus and Enterococcus faecalis, and combinations thereof, and said at least one fluorescent dye may be selected from the group consisting of xanthene-based dyes, preferably rhodamine dyes, cyanine-based dyes and DAPI and combinations thereof, preferably selected from the group consisting of Cy5™, preferably Sulfo-Cy5, MB660R and DAPI, and derivatives and combinations thereof, more preferably selected from the group consisting of Sulfo-Cy5-acid, MB660R-DBCO and DAPI, and combinations thereof. More particularly, said at least one fluorescent dye may be selected from the group consisting of xanthene-based dyes, preferably rhodamine dyes, and cyanine-based dyes, and combinations thereof, preferably selected from the group consisting of Cy5™, preferably Sulfo-Cy5, MB660R, and derivatives and combinations thereof, more preferably selected from the group consisting of Sulfo-Cy5-acid and MB660R-DBCO, and combinations thereof.

In another particular embodiment, the microorganism or group of microorganisms of interest belongs to or comprises a microorganism belonging to the group of Gram positive or negative bacteria as defined above, preferably selected from bacteria belonging to the genera Escherichia, Proteus, Pseudomonas, Staphylococcus and Enterococcus, and combinations thereof, more preferably selected from Escherichia coli, Proteus mirabilis, Pseudomonas aeruginosa, Staphylococcus aureus and Enterococcus faecalis, and combinations thereof, and said at least one fluorescent dye may be selected from the group consisting of xanthene-based dyes, preferably rhodamine dyes, cyanine-based dyes and DAPI and combinations thereof, preferably selected from the group consisting of Cy5™, preferably Sulfo-Cy5, MB660R and DAPI, and derivatives and combinations thereof, more preferably selected from the group consisting of Sulfo-Cy5-acid, MB660R-DBCO and DAPI, and combinations thereof. More particularly, said at least one fluorescent dye may be selected from the group consisting of xanthene-based dyes, preferably rhodamine dyes, and cyanine-based dyes, and combinations thereof, preferably selected from the group consisting of Cy5™, preferably Sulfo-Cy5, MB660R, and derivatives and combinations thereof, more preferably selected from the group consisting of Sulfo-Cy5-acid and MB660R-DBCO, and combinations thereof.

In another particular embodiment, the microorganism or group of microorganisms of interest belongs to or comprises a microorganism belonging to the group of heterotrophic bacteria as defined above, preferably selected from bacteria belonging to the genera Acinetobacter, Aeromonas, Brevundimonas, Burkholderia, Citrobacter, Edwardsiella, Enterobacter, Escherichia, Ochrobactrum, Klebsiella, Methylobacterium, Moraxella, Pantoea, Proteus, Pseudomonas, Ralstonia, Salmonella, Serratia, Shigella, Sphingomonas, Stenotrophomonas, Yersinia, Bacillus, Enterococcus, Micrococcus and Staphylococcus, and combinations thereof, more preferably selected from Acinetobacter baumanii, Aeromonas hydrophila, Brevundimonas diminuta, Burkholderia cepacia, Citrobacter freundii, Edwardsiella tarda, Enterobacter aerogenes, Escherichia coli, Ochrobactrum anthropic, Klebsiella pneumoniae, Methylobacterium extorquens, Moraxella osloensis, Pantoea agglomerans, Proteus mirabilis, Pseudomonas aeruginosa, Pseudomonas fluorescens, Ralstonia pickettii, Salmonella typhimurium, Serratia marcescens, Shigella sonnei, Sphingomonas paucimobilis, Stenotrophomonas maltophilia, Yersinia enterocolitica, Bacillus subtilis, Enterococcus faecalis, Micrococcus luteus (Kocuria rhizophila), Staphylococcus aureus, and combinations thereof, and said at least one fluorescent dye is selected from the group consisting of acridine dyes, xanthene-based dyes, preferably selected from the group consisting of rhodamine dyes, fluorescein dyes and carbopyronine-based dyes, anthracene-based dyes, coumarin-based dyes, cyanine-based dyes, dipyrromethene-based dyes, naphthalene-based dyes, pyrene-based dyes, squaraine-based dyes, squaraine rotaxane-based dyes and DAPI, and combinations thereof, preferably selected from the group consisting of acridine orange, DRAQ5™, Cytrak orange, Alexa Fluor™ 350, Pacific Blue, Cy5™, preferably Sulfo-Cy5, Cy5.5™, preferably Sulfo-Cy5.5, BODIPY™ dyes, preferably BODIPY 500/510, prodan, Alexa Fluor™ 405, Cascade Blue, Seta dyes, preferably Seta 650 or Seta 375, SeTau dyes, preferably SeTau 647 or SeTau 488, Alexa Fluor 488, MB660R, ATTO647 and DAPI, and derivatives and combinations thereof, more preferably selected from the group consisting of acridine orange, DRAQ5™, Cytrak orange, Alexa Fluor™ 350-NHS ester, Pacific Blue-NHS Ester, Sulfo-Cy5-acid, Sulfo-Cy5.5-acid, BODIPY 500/510, prodan, Alexa Fluor™ 405-DBCO, Cascade Blue, Seta 650-DBCO, Seta 375-NHS, SeTau 647-Maleimide, SeTau 488-NHS, Alexa Fluor 488-acid, Alexa Fluor 488-DBCO, MB660R-acid, MB660R-DBCO, ATTO647-acid and DAPI, and combinations thereof.

In particular, said at least one fluorescent dye may be selected from the group consisting of DRAQ5™, Cytrak orange, Alexa Fluor™ 350, Pacific Blue, Cy5™, preferably Sulfo-Cy5, Cy5.5™, preferably Sulfo-Cy5.5, BODIPY™ dyes, preferably BODIPY 500/510, prodan, Alexa Fluor™ 405, Cascade Blue, Seta dyes, preferably Seta 650 or Seta 375, SeTau dyes, preferably SeTau 647 or SeTau 488, Alexa Fluor 488, MB660R and ATTO647, and derivatives and combinations thereof, more preferably selected from the group consisting of DRAQ5™, Cytrak orange, Alexa Fluor™ 350-NHS ester, Pacific Blue-NHS Ester, Sulfo-Cy5-acid, Sulfo-Cy5.5-acid, BODIPY 500/510, prodan, Alexa Fluor™ 405-DBCO, Cascade Blue, Seta 650-DBCO, Seta 375-NHS, SeTau 647-Maleimide, SeTau 488-NHS, Alexa Fluor 488-acid, Alexa Fluor 488-DBCO, MB660R-acid, MB660R-DBCO and ATTO647-acid, and combinations thereof.

In a preferred embodiment, the microorganism or group of microorganisms of interest belongs to or comprises a microorganism belonging to the group of heterotrophic bacteria as defined above, preferably selected from bacteria belonging to the genera Acinetobacter, Aeromonas, Brevundimonas, Burkholderia, Citrobacter, Edwardsiella, Enterobacter, Escherichia, Ochrobactrum, Klebsiella, Methylobacterium, Moraxella, Pantoea, Proteus, Pseudomonas, Ralstonia, Salmonella, Serratia, Shigella, Sphingomonas, Stenotrophomonas, Yersinia, Bacillus, Enterococcus, Micrococcus and Staphylococcus, and combinations thereof, more preferably selected from Acinetobacter baumanii, Aeromonas hydrophila, Brevundimonas diminuta, Burkholderia cepacia, Citrobacter freundii, Edwardsiella tarda, Enterobacter aerogenes, Escherichia coli, Ochrobactrum anthropic, Klebsiella pneumoniae, Methylobacterium extorquens, Moraxella osloensis, Pantoea agglomerans, Proteus mirabilis, Pseudomonas aeruginosa, Pseudomonas fluorescens, Ralstonia pickettii, Salmonella typhimurium, Serratia marcescens, Shigella sonnei, Sphingomonas paucimobilis, Stenotrophomonas maltophilia, Yersinia enterocolitica, Bacillus subtilis, Enterococcus faecalis, Micrococcus luteus (Kocuria rhizophila), Staphylococcus aureus, and combinations thereof, and said at least one fluorescent dye is selected from the group consisting of i) a combination of Alexa Fluor 350 or a derivative thereof and Alexa Fluor 405 or a derivative thereof, and (ii) a combination MB660R or a derivative thereof and Sulfo-Cy5 or a derivative thereof, preferably selected from a combination of AF350-NHS Ester and AF405-DBCO and a combination of MB660R-DBCO and Sulfo-Cy5-acid.

In another particular embodiment, the microorganism or group of microorganisms of interest belongs to or comprises a microorganism belonging to the group of yeasts and molds as defined above, preferably selected from fungi belonging to the genera Candida, Zygosaccharomyces, Aspergillus, Saccharomyces, Geotrichum and Penicillium, and combinations thereof, more preferably selected from the group consisting of Candida albicans, Zygosaccharomyces baili, Aspergillus brasiliensis, Saccharomyces cerevisiae, Geotrichum candidum, Penicillium variotii, Penicillium chrysogenum, and combinations thereof, and said at least one fluorescent dye is selected from the group consisting of acridine dyes, xanthene-based dyes, preferably selected from the group consisting of rhodamine dyes, fluorescein dyes and carbopyronine-based dyes, coumarin-based dyes, cyanine-based dyes, dipyrromethene-based dyes, naphthalene-based dyes, pyrene-based dyes, squaraine-based dyes, squaraine rotaxane-based dyes and DAPI, and combinations thereof, preferably selected from the group consisting of Acridine orange, Alexa Fluor™ 350, Pacific Blue, Cy5™, preferably Sulfo-Cy5, prodan, Alexa Fluor™ 405, Cascade Blue, BODIPY 500/510, Seta dyes, preferably Seta 650, SeTau dyes, preferably SeTau 647 or SeTau 488, Alexa Fluor 488, MB660R and DAPI, and derivatives and combinations thereof, more preferably selected from the group consisting of Acridine orange, Alexa Fluor™ 350-NHS ester, Pacific Blue-NHS Ester, Sulfo-Cy5-acid, prodan, Alexa Fluor™ 405-DBCO, Cascade Blue, BODIPY 500/510, Seta 650-DBCO, SeTau 647-Maleimide, SeTau 488-NHS, Alexa Fluor 488-acid, MB660R-acid, MB660R-DBCO and DAPI, and combinations thereof. More particularly, said at least one fluorescent dye may be selected from the group consisting of xanthene-based dyes, preferably selected from the group consisting of rhodamine dyes, fluorescein dyes and carbopyronine-based dyes, coumarin-based dyes, cyanine-based dyes, naphthalene-based dyes, pyrene-based dyes, squaraine-based dyes, squaraine rotaxane-based dyes and DAPI, and combinations thereof, preferably selected from the group consisting of Alexa Fluor™ 350, Cy5™, preferably Sulfo-Cy5, prodan, Alexa Fluor™ 405, Seta dyes, preferably Seta 650, SeTau dyes, preferably SeTau 647, Alexa Fluor 488, MB660R, ATTO647 and DAPI, and derivatives and combinations thereof, more preferably selected from the group consisting of Alexa Fluor™ 350-NHS ester, Sulfo-Cy5-acid, prodan, Alexa Fluor™ 405-DBCO, Seta 650-DBCO, SeTau 647-Maleimide, Alexa Fluor 488-acid, MB660R-acid, MB660R-DBCO, ATTO647-acid and DAPI, and combinations thereof. Even more particularly, said at least one fluorescent dye may be selected from the group consisting of xanthene-based dyes, preferably selected from the group consisting of rhodamine dyes, fluorescein dyes and carbopyronine-based dyes, coumarin-based dyes, cyanine-based dyes, naphthalene-based dyes, pyrene-based dyes, squaraine-based dyes, squaraine rotaxane-based dyes and DAPI, and combinations thereof, preferably selected from the group consisting of Alexa Fluor™ 350, Cy5™, preferably Sulfo-Cy5, prodan, Alexa Fluor™ 405, Seta dyes, preferably Seta 650, SeTau dyes, preferably SeTau 647, Alexa Fluor 488, MB660R and DAPI, and derivatives and combinations thereof, more preferably selected from the group consisting of Alexa Fluor™ 350-NHS ester, Sulfo-Cy5-acid, prodan, Alexa Fluor™ 405-DBCO, Seta 650-DBCO, SeTau 647-Maleimide, Alexa Fluor 488-acid, MB660R-acid, MB660R-DBCO and DAPI, and combinations thereof.

In another particular embodiment, the microorganism or group of microorganisms of interest belongs to or comprises a microorganism belonging to the group of yeasts and molds as defined above, preferably selected from fungi belonging to the genera Candida, Zygosaccharomyces, Aspergillus, Saccharomyces, Geotrichum and Penicillium, and combinations thereof, more preferably selected from the group consisting of Candida albicans, Zygosaccharomyces baili, Aspergillus brasiliensis, Saccharomyces cerevisiae, Geotrichum candidum, Penicillium variotii, Penicillium chrysogenum, and combinations thereof, and said at least one fluorescent dye is selected from the group consisting of xanthene-based dyes, preferably selected from the group consisting of rhodamine dyes, fluorescein dyes and carbopyronine-based dyes, coumarin-based dyes, cyanine-based dyes, dipyrromethene-based dyes, naphthalene-based dyes, pyrene-based dyes, squaraine-based dyes and squaraine rotaxane-based dyes, and combinations thereof, preferably selected from the group consisting of Alexa Fluor™ 350, Pacific Blue, Cy5™, preferably Sulfo-Cy5, prodan, Alexa Fluor™ 405, Cascade Blue, BODIPY 500/510, Seta dyes, preferably Seta 650, SeTau dyes, preferably SeTau 647 or SeTau 488, Alexa Fluor 488 and MB660R, and derivatives and combinations thereof, more preferably selected from the group consisting of Alexa Fluor™ 350-NHS ester, Pacific Blue-NHS Ester, Sulfo-Cy5-acid, prodan, Alexa Fluor™ 405-DBCO, Cascade Blue, BODIPY 500/510, Seta 650-DBCO, SeTau 647-Maleimide, SeTau 488-NHS, Alexa Fluor 488-acid, MB660R-acid and MB660R-DBCO, and combinations thereof.

In a preferred embodiment, the microorganism or group of microorganisms of interest belongs to or comprises a microorganism belonging to the group of yeasts and molds as defined above, preferably selected from fungi belonging to the genera Candida, Zygosaccharomyces, Aspergillus, Saccharomyces, Geotrichum and Penicillium, and combinations thereof, more preferably selected from the group consisting of Candida albicans, Zygosaccharomyces baili, Aspergillus brasiliensis, Saccharomyces cerevisiae, Geotrichum candidum, Penicillium variotii, Penicillium chrysogenum, and combinations thereof, and said at least one fluorescent dye is selected from the group consisting of MB660R and derivatives, and combinations thereof, more preferably is MB660R-DBCO.

In another particular embodiment, the microorganism or group of microorganisms of interest comprises
one or several bacteria, preferably selected from the groups, as defined above, of thermophilic acidophilic bacteria (TAB), acetic acid bacteria (AAB), lactic acid bacteria (LAB), anaerobic bacteria, aerobic mesophilic bacteria, Gram negative bacteria and Gram positive bacteria, and combinations thereof, and
one or several microscopic fungi, preferably selected from yeasts and molds as defined above, and
said at least one fluorescent dye is selected from the group consisting of xanthene-based dyes, preferably selected from the group consisting of rhodamine dyes, fluorescein dyes and carbopyronine-based dyes, coumarin-based dyes, cyanine-based dyes, pyrene-based dyes, squaraine-based dyes and squaraine rotaxane-based dyes, and combinations thereof, preferably selected from the group consisting of Alexa Fluor™ 350, Cy5™, preferably Sulfo-Cy5, Alexa Fluor™ 405, Seta dyes, preferably Seta 650, SeTau dyes, preferably SeTau 647, Alexa Fluor 488 and MB660R, and derivatives and combinations thereof, more preferably selected from the group consisting of Alexa Fluor™ 350-NHS ester, Sulfo-Cy5-acid, Alexa Fluor™ 405-DBCO, Seta 650-DBCO, SeTau 647-Maleimide, Alexa Fluor 488-acid, MB660R-acid and MB660R-DBCO, and combinations thereof, even more preferably selected from the group consisting of Sulfo-Cy5-acid and MB660R-DBCO, and combinations thereof. In particular, said at least one fluorescent dye may be a combination of Sulfo-Cy5-acid and MB660R-DBCO.

Preferably, in this embodiment, the microorganism or group of microorganisms of interest is selected from the group consisting of bacteria belonging to the genera Alicyclobacillus (e.g. Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans), Acetobacter (e.g. Acetobacter aceti), Gluconoacetobacter (e.g. Gluconoacetobacter liquefaciens), Gluconobacter (e.g. Gluconobacter oxydans), Asaia (e.g. Asaia siamensis), Lactobacillus (e.g. Lactobacillus casei, Lactobacillus plantarum), Weissella (e.g. Weissella confusa), Clostridium (e.g. Clostridium sporogenes), Cutibacterium (e.g Cutibacterium acnes), Methylobacterium (e.g. Methylobacterium extorquens), Pseudomonas (e.g. Pseudomonas aeruginosa), Bacillus (e.g. Bacillus subtilis), Escherichia (e.g. Escherichia coli), Staphylococcus (e.g. Staphylococcus aureus), Acinetobacter (e.g. Acinetobacter baumannii), Cronobacter (e.g. Cronobacter sakazakii), Klebsiella (e.g. Klebsiella pneumoniae), Salmonella (e.g. Salmonella typhimurium), Enterococcus (e.g. Enterococcus faecalis), Listeria (e.g. Listeria grayi), Shigella (e.g. Shigella sonnei), Kocuria (e.g. Kocuria rhizophila), Burkholderia (e.g. Burkholderia cepacia), and fungi belonging to the genera Candida (e.g. Candida albicans), Zygosaccharomyces (e.g. Zygosaccharomyces bailii), Aspergillus (e.g. Aspergillus brasiliensis), Geotrichum (Geotrichum candidum), Saccharomyces (e.g. Saccharomyces cerevisiae) and Penicillium (e.g. Penicillium variotii, Penicillium chrysogenum), and combinations thereof.

More preferably, in this embodiment, the microorganism or group of microorganisms of interest is selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Asaia siamensis, Lactobacillus casei, Lactobacillus plantarum, Weissella confusa, Clostridium sporogenes, Cutibacterium acnes, Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Listeria grayi, Shigella sonnei, Kocuria rhizophila, Burkholderia cepacia, Candida albicans, Zygosaccharomyces baili, Aspergillus brasiliensis, Saccharomyces cerevisiae, Geotrichum candidum, Penicillium variotii and Penicillium chrysogenum, and combinations thereof.

More preferably, in this embodiment, the microorganism or group of microorganisms of interest is selected from the group consisting of bacteria belonging to the genera Weissella (e.g. Weissella confusa), Cutibacterium (e.g Cutibacterium acnes), Methylobacterium (e.g. Methylobacterium extorquens), Enterococcus (e.g. Enterococcus faecalis), and fungi belonging to the genera Candida (e.g. Candida albicans) and Aspergillus (e.g. Aspergillus brasiliensis), and combinations thereof, in particular is selected from the group consisting of Weissella confusa, Cutibacterium acnes, Methylobacterium extorquens, Enterococcus faecalis, Candida albicans and Aspergillus brasiliensis, and combinations thereof.

In another particular embodiment, the microorganism or group of microorganisms of interest comprises
one or several bacteria, preferably selected from the groups, as defined above, of thermophilic acidophilic bacteria (TAB), acetic acid bacteria (AAB), lactic acid bacteria (LAB), anaerobic bacteria, aerobic mesophilic bacteria, Gram negative bacteria and Gram positive bacteria, heterotrophic bacteria, and combinations thereof, and
one or several microscopic fungi, preferably selected from yeasts and molds as defined above, and
said at least one fluorescent dye is selected from the group consisting of xanthene-based dyes, preferably selected from the group consisting of rhodamine dyes, fluorescein dyes and carbopyronine-based dyes, coumarin-based dyes, cyanine-based dyes, pyrene-based dyes, squaraine-based dyes and squaraine rotaxane-based dyes, and combinations thereof, preferably selected from the group consisting of Alexa Fluor™ 350, Pacific Blue™, Cy5™, preferably Sulfo-Cy5, Alexa Fluor™ 405, Cascade Blue™, Seta dyes, preferably Seta 650, SeTau dyes, preferably SeTau 647 or SeTau 488, Alexa Fluor 488 and MB660R, and derivatives and combinations thereof, more preferably selected from the group consisting of Alexa Fluor™ 350-NHS ester, Pacific Blue™-NHS ester, Sulfo-Cy5-acid, Alexa Fluor™ 405-DBCO, Cascade Blue™, Seta 650-DBCO, SeTau 488-NHS, SeTau 647-Maleimide, Alexa Fluor 488-acid, MB660R-acid and MB660R-DBCO, and combinations thereof, even more preferably selected from the group consisting of Sulfo-Cy5-acid and MB660R-DBCO, and combinations thereof. In particular, said at least one fluorescent dye may be a combination of Sulfo-Cy5-acid and MB660R-DBCO.

Preferably, in this embodiment, the microorganism or group of microorganisms of interest is selected from the group consisting of bacteria belonging to the genera Alicyclobacillus (e.g. Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans), Acetobacter (e.g. Acetobacter aceti), Gluconoacetobacter (e.g. Gluconoacetobacter liquefaciens), Gluconobacter (e.g. Gluconobacter oxydans), Asaia (e.g. Asaia siamensis), Lactobacillus (e.g. Lactobacillus casei, Lactobacillus plantarum), Weissella (e.g. Weissella confusa), Clostridium (e.g. Clostridium sporogenes), Cutibacterium (e.g Cutibacterium acnes), Methylobacterium (e.g. Methylobacterium extorquens), Pseudomonas (e.g. Pseudomonas aeruginosa), Bacillus (e.g. Bacillus subtilis), Escherichia (e.g. Escherichia coli), Staphylococcus (e.g. Staphylococcus aureus), Acinetobacter (e.g. Acinetobacter baumannii), Cronobacter (e.g. Cronobacter sakazakii), Klebsiella (e.g. Klebsiella pneumoniae), Salmonella (e.g. Salmonella typhimurium), Enterococcus (e.g. Enterococcus faecalis), Listeria (e.g. Listeria grayi), Shigella (e.g. Shigella sonnei), Kocuria (e.g. Kocuria rhizophila), Burkholderia (e.g. Burkholderia cepacia), Aeromonas (e.g. Aeromonas hydrophila), Brevundimonas (e.g. Brevundimonas diminuta), Citrobacter (e.g. Citrobacter freundii), Edwardsiella (e.g. Edwardsiella tarda), Enterobacter (e.g. Enterobacter aerogenes), Ochrobactrum (e.g. Ochrobactrum anthropic), Moraxella (e.g. Moraxella osloensis), Pantoea (e.g. Pantoea agglomerans), Proteus (e.g. Proteus mirabilis), Ralstonia (e.g. Ralstonia pickettii), Serratia (e.g. Serratia marcescens), Sphingomonas (e.g. Sphingomonas paucimobilis), Stenotrophomonas (e.g. Stenotrophomonas maltophilia), Vibrio (e.g Vibrio parahaemolyticus), Yersinia (e.g. Yersinia enterocolitica), and combinations thereof.

More preferably, in this embodiment, the microorganism or group of microorganisms of interest is selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Asaia siamensis, Lactobacillus casei, Lactobacillus plantarum, Weissella confusa, Clostridium sporogenes, Cutibacterium acnes, Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Listeria grayi, Shigella sonnei, Kocuria rhizophila, Burkholderia cepacia, Aeromonas hydrophila, Brevundimonas diminuta, Citrobacter freundii, Edwardsiella tarda, Enterobacter aerogenes, Ochrobactrum anthropic, Moraxella osloensis, Pantoea agglomerans, Proteus mirabilis, Ralstonia pickettii, Serratia marcescens, Sphingomonas paucimobilis, Stenotrophomonas maltophilia, Vibrio parahaemolyticus, Yersinia enterocolitica, Candida albicans, Zygosaccharomyces bailii, Aspergillus brasiliensis, Saccharomyces cerevisiae, Geotrichum candidum, Penicillium variotii and Penicillium chrysogenum, and combinations thereof.

Even more preferably, in this embodiment, the microorganism or group of microorganisms of interest is selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Asaia siamensis, Lactobacillus casei, Lactobacillus plantarum, Weissella confusa, Clostridium sporogenes, Cutibacterium acnes, Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Shigella sonnei, Kocuria rhizophila, Burkholderia cepacia, Aeromonas hydrophila, Brevundimonas diminuta, Citrobacter freundii, Edwardsiella tarda, Enterobacter aerogenes, Ochrobactrum anthropic, Moraxella osloensis, Pantoea agglomerans, Proteus mirabilis, Ralstonia pickettii, Serratia marcescens, Sphingomonas paucimobilis, Stenotrophomonas maltophilia, Yersinia enterocolitica, Candida albicans, Zygosaccharomyces bailii, Aspergillus brasiliensis, Saccharomyces cerevisiae, Geotrichum candidum, Penicillium variotii and Penicillium chrysogenum, and combinations thereof.

It is herein disclosed any embodiment of the method of the invention
wherein the microorganism is a microorganism A as defined in Table 1 or wherein the group of microorganisms of interest comprises a microorganism A as defined in Table 1, and
wherein said at least one fluorescent dye is a fluorescent dye B as defined in Table 1.

In embodiments wherein the sample is filtered on a filtration membrane, said filtration membrane is preferably a membrane C as defined in Table 1.

Each combination of microorganism, fluorescent dye(s) and, optionally, membrane of Table 1 is specifically disclosed. Thus, as illustration, it is herein specifically disclosed the use of Alexa Fluor 488-Acid to detect or enumerate Alicyclobacillus acidoterrestris in a sample, optionally after filtration on a PVDF membrane, or the use of DAPI to detect or enumerate Acetobacter aceti in a sample, optionally after filtration on a MCE membrane.

TABLE 1
Examples of fluorescent dyes useful to detect microorganisms

| | | Optional |
|---|---|---|
| Microorganism A | Fluorescent dye B | membrane C |
| Alicyclobacillus | Alexa Fluor 488-Acid | PVDF |
| acidoterrestris | Alexa Fluor 488-DBCO | PVDF |
| | MB660R-Acid | PVDF |
| | MB660R-DBCO | PVDF |
| | ATTO647-Acid | PVDF |
| Alicyclobacillus | MB660R-DBCO | PVDF or MCE |
| acidocaldarius | | |
| Alicyclobacillus | Alexa Fluor 488-Acid | PVDF |
| acidiphilus | Alexa Fluor 488-DBCO | PVDF |
| | MB660R-Acid | PVDF |
| | MB660R-DBCO | PVDF or MCE |
| | ATTO647-Acid | PVDF |
| Alicyclobacillus | MB660R-Acid | PVDF |
| cycloheptanicus | MB660R-DBCO | PVDF |
| Alicyclobacillus | MB660R-Acid | PVDF |
| hesperidum | MB660R-DBCO | PVDF |
| Alicyclobacillus | MB660R-Acid | PVDF |
| herbarius | MB660R-DBCO | PVDF |
| Alicyclobacillus | MB660R-Acid | PVDF |
| contaminans | MB660R-DBCO | PVDF |
| Acetobacter aceti | DAPI | MCE |
| | Alexa Fluor 488-Acid | PVDF |
| | Alexa Fluor 488-DBCO | PVDF |
| | MB660R-Acid | PVDF |
| | MB660R-DBCO | PVDF |
| | ATTO647-Acid | PVDF |
| Gluconoacetobacter | DAPI | MCE |
| liquefaciens | Alexa Fluor 488-Acid | PVDF |
| | Alexa Fluor 488-DBCO | PVDF |
| | MB660R-Acid | PVDF |
| | MB660R-DBCO | PVDF |
| | ATTO647-Acid | PVDF |
| Gluconobacter | DAPI | MCE |
| oxydans | Alexa Fluor 488-Acid | PVDF |
| | MB660R-Acid | PVDF |
| | MB660R-DBCO | PVDF |
| | ATTO647-Acid | PVDF |
| Asaia siamensis | Alexa Fluor 488-Acid | PVDF |
| | Alexa Fluor 488-DBCO | PVDF |
| | MB660R-Acid | PVDF |
| | ATTO647-Acid | PVDF |
| Lactobacillus casei | DAPI | MCE |
| | Alexa Fluor 488-Acid | PVDF |
| | Alexa Fluor 488-DBCO | PVDF |
| | MB660R-Acid | PVDF |
| Lactobacillus | DAPI | MCE |
| plantarum | Alexa Fluor 488-Acid | PVDF |
| | Alexa Fluor 488-DBCO | PVDF |
| | MB660R-Acid | PVDF |
| | MB660R-DBCO | PVDF |
| | ATTO647-Acid | PVDF |
| Weissella confusa | DAPI | MCE |
| | Alexa Fluor 488-Acid | PVDF |
| | MB660R-Acid | PVDF |
| | MB660R-DBCO | PVDF |
| | ATTO647-Acid | PVDF |
| Zygosaccharomyces | DAPI | MCE |
| bailii | AF488-Acid | PVDF or MCE |
| | MB660R-Acid | PVDF or MCE |
| | MB660R-DBCO | PVDF |
| Aspergillus | AF350-NHS ester | PVDF or MCE |
| brasiliensis | Pacific Blue-NHS Ester | MCE |
| | Sulfo-Cy5-Acid | PVDF or MCE |
| | Prodan | PVDF or MCE |
| | AF405-DBCO | PVDF or MCE |
| | Cascade Blue | MCE |
| | Seta 650-DBCO | PVDF |
| | SeTau 488-NHS | PVDF |
| | SeTau 647-Maleimide | PVDF |
| | AF488-Acid | PVDF |
| | MB660R-Acid | PVDF |
| | MB660R-DBCO | PVDF |
| | Acridine orange | MCE |
| | BODIPY 500/510 | MCE |
| Candida albicans | AF350-NHS ester | PVDF or MCE |
| | Pacific Blue-NHS Ester | MCE |
| | Sulfo-Cy5-Acid | PVDF or MCE |
| | Prodan | PVDF or MCE |
| | AF405-DBCO | PVDF or MCE |
| | Cascade Blue | MCE |
| | Seta 650-DBCO | PVDF |
| | Seta 375-NHS | MCE |
| | SeTau 488-NHS | PVDF |
| | SeTau 647-Maleimide | PVDF |
| | MB660R-DBCO | PVDF |
| | BODIPY 500/510 | PVDF or MCE |
| Saccharomyces | DAPI | MCE |
| cerevisiae | AF488-Acid | PVDF or MCE |
| | MB660R-Acid | PVDF or MCE |
| | MB660R-DBCO | PVDF |
| Penicillium variotii | MB660R-DBCO | PVDF |
| Penicillium chrysogenum | MB660R-DBCO | PVDF |
| Geotrichum candidum | MB660R-DBCO | PVDF |
| Cutibacterium acnes | MB660R-DBCO and Sulfo-CY5-acid | PVDF |
| Clostridium sporogenes | MB660R-DBCO and Sulfo-CY5-acid | PVDF |
| | DAPI | MCE |
| Methylobacterium | Sulfo Cy5-acid | PVDF |
| extorquens | DAPI | MCE |
| | MB660R-DBCO | PVDF or MCE |
| | AF350-NHS Ester and AF405-DBCO | PVDF |
| Pseudomonas aeruginosa | Sulfo Cy5-acid | PVDF |
| | DAPI | PVDF or MCE |
| | MB660R-DBCO | PVDF |
| | AF350-NHS Ester and AF405-DBCO | PVDF |
| Bacillus subtilis | Sulfo Cy5-acid | PVDF |
| | MB660R-DBCO | PVDF |
| | AF350-NHS Ester and AF405-DBCO | PVDF |
| Escherichia coli | Acridine orange | MCE |
| | Cytrak orange | MCE |
| | DRAQ5 | PVDF or MCE |
| | AF350-NHS Ester | PVDF or MCE |
| | Pacific Blue-NHS Ester | MCE |
| | Sulfo-Cy5.5 acid | MCE |
| | Sulfo-Cy5-Acid | PVDF or MCE |
| | BODIPY 500/510 | PVDF or MCE |
| | DAPI | MCE |
| | Prodan | PVDF or MCE |
| | AF405-DBCO | MCE |
| | Cascade Blue | MCE |
| | Seta 650-DBCO | PVDF |
| | Seta 375-NHS | MCE |
| | SeTau 488-NHS | PVDF |
| | SeTau 647-Maleimide | PVDF or MCE |
| | Alexa Fluor 488-Acid | PVDF or MCE |
| | Alexa Fluor 488-DBCO | PVDF |
| | MB660R-Acid | PVDF or MCE |
| | MB660R-DBCO | PVDF |
| | ATTO647-Acid | PVDF |
| Staphylococcus aureus | Sulfo-Cy5-Acid | PVDF |
| | DAPI | MCE |
| | Alexa Fluor 488-Acid | PVDF |
| | MB660R-Acid | PVDF |
| | MB660R-DBCO | PVDF |
| | ATTO647-Acid | PVDF |
| | AF350-NHS Ester and AF405-DBCO | PVDF |
| Acinetobacter | MB660R-DBCO | PVDF |
| baumannii | AF350-NHS Ester and AF405-DBCO | PVDF |
| | MB660R-DBCO and Sulfo-Cy5-acid | PVDF |
| Cronobacter sakazakii | MB660R-DBCO | PVDF |
| Klebsiella pneumoniae | MB660R-DBCO | PVDF |
| | MB660R-DBCO and Sulfo-Cy5-acid | PVDF |
| | DAPI | MCE |
| Salmonella typhimurium | MB660R-DBCO | PVDF |
| Enterococcus faecalis | DRAQ5 | PVDF or MCE |
| | Sulfo-Cy5-Acid | PVDF or MCE |
| | MB660R-DBCO | PVDF |
| | DAPI | MCE |
| Shigella sonnei | MB660R-DBCO | PVDF |
| | MB660R-DBCO and Sulfo-Cy5-acid | PVDF |
| Kocuria rhizophila | MB660R-DBCO | PVDF |
| Burkholderia cepacia | DAPI | MCE |
| | MB660R-DBCO | PVDF |
| | AF350-NHS Ester and AF405-DBCO | PVDF or MCE |
| | MB660R-DBCO and Sulfo-Cy5-acid | PVDF |
| Ralstonia pickettii | AF350-NHS Ester and AF405-DBCO | PVDF |
| | MB660R-DBCO and Sulfo-Cy5-acid | PVDF |
| Brevundimonas diminuta | AF350-NHS Ester and AF405-DBCO | PVDF |
| | MB660R-DBCO and Sulfo-Cy5-acid | PVDF |
| Aeromonas hydrophila | AF350-NHS Ester and AF405-DBCO | PVDF |
| | MB660R-DBCO and Sulfo-Cy5-acid | PVDF |
| Pseudomonas fluorescens | MB660R-DBCO and Sulfo-Cy5-acid | PVDF |
| Citrobacter freundii | MB660R-DBCO and Sulfo-Cy5-acid | PVDF |
| Enterobacter aerogenes | MB660R-DBCO and Sulfo-Cy5-acid | PVDF |
| Pantoea agglomerans | MB660R-DBCO and Sulfo-Cy5-acid | PVDF |
| Stenotrophomonas | MB660R-DBCO and Sulfo-Cy5-acid | PVDF |
| maltophilia | | |
| Serratia marcescens | MB660R-DBCO and Sulfo-Cy5-acid | PVDF |
| Yersinia | MB660R-DBCO and Sulfo-Cy5-acid | PVDF |
| enterocolitica | | |
| Edwardsiella tarda | MB660R-DBCO and Sulfo-Cy5-acid | PVDF |
| Moraxella osloensis | MB660R-DBCO and Sulfo-Cy5-acid | PVDF |
| Ochrobactrum anthropi | MB660R-DBCO and Sulfo-Cy5-acid | PVDF |
| Proteus mirabilis | MB660R-DBCO and Sulfo-Cy5-acid | PVDF |
| | DAPI | MCE |
| Sphingomonas | MB660R-DBCO and Sulfo-Cy5-acid | PVDF |
| paucimobilis | | |
| Salmonella typhimurium | MB660R-DBCO and Sulfo-Cy5-acid | PVDF |

In some embodiments, the microorganism or group of microorganisms of interest belongs to or comprises a microorganism selected from the group consisting of Alicyclobacillus acidoterrestris, Alicyclobacillus acidiphilus, Acetobacter aceti, Gluconoacetobacter liquefaciens, Gluconobacter oxydans, Asaia siamensis, Lactobacillus casei, Lactobacillus plantarum, Weissella confusa, Zygosaccharomyces baii, Aspergillus brasiliensis, Saccharomyces cerevisiae, Escherichia coli and Staphylococcus aureus, and combinations thereof, and said at least one fluorescent dye is Alexa Fluor 488 or a derivative thereof, preferably Alexa Fluor 488-Acid.

In some embodiments, the microorganism or group of microorganisms of interest belongs to or comprises a microorganism selected from the group consisting of Alicyclobacillus acidoterrestris, Alicyclobacillus acidiphilus, Acetobacter aceti, Gluconoacetobacter liquefaciens, Asaia siamensis, Lactobacillus casei, Lactobacillus plantarum and Escherichia coli, and combinations thereof, and said at least one fluorescent dye is Alexa Fluor 488 or a derivative thereof, preferably Alexa Fluor 488-DBCO.

In some embodiments, the microorganism or group of microorganisms of interest belongs to or comprises a microorganism selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Asaia siamensis, Lactobacillus casei, Lactobacillus plantarum, Weissella confusa, Zygosaccharomyces bailii, Aspergillus brasiliensis, Candida albicans, Saccharomyces cerevisiae, Penicillium variotii, Penicillium chrysogenum, Geotrichum candidum, Cutibacterium acnes, Clostridium sporogenes, Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Shigella sonnei, Kocuria rhizophila, Burkholderia cepacia, Ralstonia pickettii, Brevundimonas diminuta, Aeromonas hydrophila, Pseudomonas fluorescens, Citrobacter freundii, Enterobacter aerogenes, Pantoea agglomerans, Stenotrophomonas maltophilia, Serratia marcescens, Yersinia enterocolitica, Edwardsiella tarda, Moraxella osloensis, Ochrobactrum anthropi, Proteus mirabilis, Sphingomonas paucimobilis and Salmonella typhimurium, and combinations thereof, and said at least one fluorescent dye is MB660R or a derivative thereof, preferably MB660R-acid and/or MB660R-DBCO.

In some embodiments, the microorganism or group of microorganisms of interest belongs to or comprises a microorganism selected from the group consisting of Alicyclobacillus acidoterrestris, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Asaia siamensis, Lactobacillus casei, Lactobacillus plantarum, Weissella confusa, Zygosaccharomyces bailii, Aspergillus brasiliensis, Saccharomyces cerevisiae, Escherichia coli and Staphylococcus aureus, and combinations thereof, and said at least one fluorescent dye is MB660R or a derivative thereof, preferably MB660R-acid.

In some embodiments, the microorganism or group of microorganisms of interest belongs to or comprises a microorganism selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Lactobacillus plantarum, Weissella confusa, Zygosaccharomyces bailii, Aspergillus brasiliensis, Candida albicans, Saccharomyces cerevisiae, Penicillium variotii, Penicillium chrysogenum, Geotrichum candidum, Cutibacterium acnes, Clostridium sporogenes, Methylobacterium extorquens, Pseudomonas aeruginosa, Bacillus subtilis, Escherichia coli, Staphylococcus aureus, Acinetobacter baumannii, Cronobacter sakazakii, Klebsiella pneumoniae, Salmonella typhimurium, Enterococcus faecalis, Shigella sonnei, Kocuria rhizophila, Burkholderia cepacia, Ralstonia pickettii, Brevundimonas diminuta, Aeromonas hydrophila, Pseudomonas fluorescens, Citrobacter freundii, Enterobacter aerogenes, Pantoea agglomerans, Stenotrophomonas maltophilia, Serratia marcescens, Yersinia enterocolitica, Edwardsiella tarda, Moraxella osloensis, Ochrobactrum anthropi, Proteus mirabilis, Sphingomonas paucimobilis and Salmonella typhimurium, and combinations thereof, and said at least one fluorescent dye is MB660R or a derivative thereof, preferably MB660R-DBCO. Optionally, said at least one fluorescent dye further comprises Cy5, preferably Sulfo Cy-5, or a derivative thereof, preferably Sulfo Cy5-acid.

In some embodiments, the microorganism or group of microorganisms of interest belongs to or comprises a microorganism selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius, Alicyclobacillus contaminans, Acetobacter aceti, Gluconoacetobacter liquefaciens, Gluconobacter oxydans, Lactobacillus plantarum, Weissella confusa, Zygosaccharomyces bailii, Aspergillus brasiliensis, Candida albicans, Saccharomyces cerevisiae, Penicillium variotii, Penicillium chrysogenum, Geotrichum candidum, Methylobacterium extorquens, Pseudomonas aeruginosa, Bacillus subtilis, Escherichia coli, Staphylococcus aureus, Acinetobacter baumannii, Cronobacter sakazakii. Klebsiella pneumoniae. Salmonella typhimurium. Enterococcus faecalis. Shigella sonnei, Kocuria rhizophila and Burkholderia cepacia, and combinations thereof, and said at least one fluorescent dye is MB660R or a derivative thereof, preferably MB660R-DBCO.

In some embodiments, the microorganism or group of microorganisms of interest belongs to or comprises a microorganism selected from the group consisting of Cutibacterium acnes, Clostridium sporogenes, Acinetobacter baumannii, Klebsiella pneumoniae, Shigella sonnei, Burkholderia cepacia, Ralstonia pickettii, Brevundimonas diminuta, Aeromonas hydrophila, Pseudomonas fluorescens, Citrobacter freundii, Enterobacter aerogenes, Pantoea agglomerans, Stenotrophomonas maltophilia, Serratia marcescens, Yersinia enterocolitica, Edwardsiella tarda, Moraxella osloensis, Ochrobactrum anthropi, Proteus mirabilis, Sphingomonas paucimobilis and Salmonella typhimurium, and combinations thereof, and said at least one fluorescent dye is a combination of MB660R or a derivative thereof, and Cy5, preferably Sulfo Cy-5, or a derivative thereof, preferably a combination of MB660R-DBCO and Sulfo Cy5-acid.

In some embodiments, the microorganism or group of microorganisms of interest belongs to or comprises a microorganism selected from the group consisting of Alicyclobacillus acidoterrestris, Alicyclobacillus acidiphilus, Acetobacter aceti, Gluconoacetobacter liquefaciens, Gluconobacter oxydans, Asaia siamensis, Lactobacillus plantarum, Weissella confusa, Escherichia coli and Staphylococcus aureus, and combinations thereof, and said at least one fluorescent dye is ATTO647 or a derivative thereof, preferably ATTO647-acid.

In some embodiments, the microorganism or group of microorganisms of interest belongs to or comprises a microorganism selected from the group consisting of Acetobacter aceti, Gluconoacetobacter liquefaciens, Gluconobacter oxydans, Lactobacillus casei, Lactobacillus plantarum, Weissella confusa, Zygosaccharomyces bailii, Saccharomyces cerevisiae, Clostridium sporogenes, Methylobacterium extorquens, Pseudomonas aeruginosa, Escherichia coli, Staphylococcus aureus, Klebsiella pneumoniae, Enterococcus faecalis, Burkholderia cepacia and Proteus mirabilis, and combinations thereof, and said at least one fluorescent dye is DAPI.

In some embodiments, the microorganism or group of microorganisms of interest belongs to or comprises a microorganism selected from the group consisting of Aspergillus brasiliensis, Candida albicans, Methylobacterium extorquens, Pseudomonas aeruginosa, Bacillus subtilis, Escherichia coli, Staphylococcus aureus, Acinetobacter baumannii, Burkholderia cepacia, Ralstonia pickettii, Brevundimonas diminuta and Aeromonas hydrophila, and combinations thereof, and said at least one fluorescent dye is Alexa Fluor 350 or a derivative thereof, preferably Alexa Fluor 350-NHS ester. Optionally, said at least one fluorescent dye further comprises Alexa Fluor 405 or a derivative thereof, preferably Alexa Fluor 405-DBCO.

In some embodiments, the microorganism or group of microorganisms of interest belongs to or comprises a microorganism selected from the group consisting of Aspergillus brasiliensis, Candida albicans, Cutibacterium acnes, Clostridium sporogenes, Methylobacterium extorquens, Pseudomonas aeruginosa, Bacillus subtilis, Escherichia coli, Staphylococcus aureus, Acinetobacter baumannii, Klebsiella pneumoniae, Enterococcus faecalis, Shigella sonnei, Burkholderia cepacia, Ralstonia pickettii, Brevundimonas diminuta, Aeromonas hydrophila, Pseudomonas fluorescens, Citrobacter freundii, Enterobacter aerogenes, Pantoea agglomerans, Stenotrophomonas maltophilia, Serratia marcescens, Yersinia enterocolitica, Edwardsiella tarda, Moraxella osloensis, Ochrobactrum anthropi, Proteus mirabilis, Sphingomonas paucimobilis and Salmonella typhimurium, and combinations thereof, and said at least one fluorescent dye is Cy5, preferably Sulfo-Cy5, or a derivative thereof, preferably Sulfo Cy5-acid. Optionally, said at least one fluorescent dye further comprises MB660R or a derivative thereof, preferably MB660R-DBCO.

In some embodiments, the microorganism or group of microorganisms of interest belongs to or comprises a microorganism selected from the group consisting of Aspergillus brasiliensis, Candida albicans, Methylobacterium extorquens, Pseudomonas aeruginosa, Bacillus subtilis, Escherichia coli, Staphylococcus aureus and Enterococcus faecalis, and combinations thereof, and said at least one fluorescent dye is Cy5, preferably Sulfo-Cy5, or a derivative thereof, preferably Sulfo Cy5-acid.

In some embodiments, the microorganism or group of microorganisms of interest belongs to or comprises Escherichia coli, and said at least one fluorescent dye is Cy5.5, preferably Sulfo-Cy5.5, or a derivative thereof, preferably Sulfo Cy5.5-acid.

In some embodiments, the microorganism or group of microorganisms of interest belongs to or comprises a microorganism selected from the group consisting of Aspergillus brasiliensis, Candida albicans, Methylobacterium extorquens, Pseudomonas aeruginosa, Bacillus subtilis, Escherichia coli, Staphylococcus aureus, Acinetobacter baumannii, Burkholderia cepacia, Ralstonia pickettii, Brevundimonas diminuta and Aeromonas hydrophila, and combinations thereof, and said at least one fluorescent dye is Alexa Fluor 405 or a derivative thereof, preferably Alexa Fluor 405-DBCO. Optionally, said at least one fluorescent dye further comprises Alexa Fluor 350 or a derivative thereof, preferably Alexa Fluor 350-NHS ester.

In some embodiments, the microorganism or group of microorganisms of interest belongs to or comprises a microorganism selected from the group consisting of Aspergillus brasiliensis and Escherichia coli, and combinations thereof, and said at least one fluorescent dye is Acridine orange.

In some embodiments, the microorganism or group of microorganisms of interest belongs to or comprises a microorganism selected from the group consisting of Aspergillus brasiliensis, Candida albicans and Escherichia coli, and combinations thereof, and said at least one fluorescent dye is selected from the group consisting of Bodipy 500/510; Alexa Fluor 350 or a derivative thereof, preferably Alexa Fluor 350-NHS ester; Pacific blue or a derivative thereof, preferably Pacific blue-NHS ester; prodan; Alexa Fluor 405 or a derivative thereof, preferably Alexa Fluor 405-DBCO; Cascade Blue; Seta 650 or a derivative thereof, preferably Seta 650-DBCO; Setau 488 or a derivative thereof, preferably Setau 488-NHS; Setau 647 or a derivative thereof, preferably Setau 647-maleimide, and any combination thereof.

In some embodiments, the microorganism or group of microorganisms of interest belongs to or comprises a microorganism selected from the group consisting of Candida albicans and Escherichia coli, and combination thereof, and said at least one fluorescent dye is Seta 375 or a derivative thereof, preferably Seta 375-NHS.

In some embodiments, the microorganism or group of microorganisms of interest belongs to or comprises a microorganism selected from the group consisting of Escherichia coli and Enterococcus faecalis, and combination thereof, and said at least one fluorescent dye is DRAQ5.

In some embodiments, the microorganism or group of microorganisms of interest belongs to or comprises Escherichia coli and said at least one fluorescent dye is Cytrak Orange.

A solution comprising the fluorescent dye(s) may be incorporated in the growth medium before pouring said medium in the contained or may be poured or streaked on the surface of the solid growth medium. Preferably, the fluorescent dye(s) are poured or streaked on the surface of the solid growth medium before or after contacting said growth medium with the sample. In embodiments wherein the sample has been concentrated on a membrane filter before step a), the fluorescent dye(s) may be poured (uniformly or as drops) on the surface of the solid growth medium and the membrane filter may be then placed on the surface of said growth medium, thereby contacting both growth medium and the fluorescent dye(s). Alternatively, the fluorescent dye(s) may be added to the sample prior to filtration/concentration on the membrane or may be filtered on the same membrane prior or after the filtration of the sample, preferably after the filtration of the sample. The membrane filter may be then placed on the surface of said growth medium.

The concentration and volume of each fluorescent dye may be easily adjusted by the skilled person. Typically, the fluorescent dyes are used at a concentration ranging from 1 μM to 1 mM, preferably ranging from 1 μM to 500 μM, preferably ranging from 5 μM to 250 μM. In embodiments wherein the fluorescent dye(s) is poured on the surface of the solid growth medium and a membrane filter (47 mm) is then placed on the surface of said growth medium, the volume of the drop containing the fluorescent dye(s) is typically between 100 μL and 300 μL, preferably between 150 μL and 200 μL.

### Conditions and Time of Incubation

In step b) of the method of the invention, the container comprising the sample, the growth medium and at least one fluorescent dye as defined above, is incubated under conditions and for a time sufficient to form microcolonies of the microorganism or group of microorganisms of interest. The growth of microorganisms is thus conducted in the presence of the fluorescent dye(s).

As used herein, the term “microcolonies” refers to colonies which have grown for several hours or days depending of the microorganism and which are invisible or barely visible with the naked eye. Typically, the size of microcolonies is less than 500 μm, preferably between 10 μm to 200 μm.

Incubation time required to obtain microcolonies depends on the microorganisms and in particular on the generation time of microorganisms. This incubation time can be easily adjusted by the skilled person depending on the microorganism or group of microorganisms of interest, and parameters impacting the generation time such as the temperature and the type of growth medium. Typically, the incubation time is ranged from 3 h to 96 h. The surface of the growth medium, or the membrane filter, may be periodically microscopically monitored for growth using a fluorescent microscope or another suitable system, at one or more specific wavelengths, in order to adjust time incubation.

Similarly, conditions of incubation, in particular atmosphere and temperature conditions, also depend on said microorganisms. These conditions may be adapted in order to favour the microorganism or group of microorganisms of interest and/or limit contamination by other microorganisms.

In particular, the incubation may be conducted under aerobic, semi-aerobic (typically between 2 and 10% oxygen), or anaerobic conditions. In semi-aerobic or anaerobic conditions, the concentration of carbon dioxide may be elevated in order to favour capnophile microorganisms, typically to reach a value between 1 and 20% carbon dioxide.

The temperature typically ranges between 15° C. and 50° C., preferably between 20° C. and 45° C. However, extremophile microorganisms may be cultured at temperatures below 15° C. or higher than 50° C. The incubation temperature can be easily adjusted by the skilled person depending on the microorganism or group of microorganisms of interest. Thermophilic microorganisms such as TAB are usually cultured between 37° C. and 48° C., preferably about 45° C. Mesophilic microorganisms are usually cultured between 30° C. and 40° C., preferably about 35° C. Acid acetic bacteria and microscopic fungi are usually cultured between 20° C. and 30° C., preferably about 25° C. Lactic acid bacteria are usually cultured between 20° C. and 40° C., preferably about 30° C.

In embodiments wherein the microorganism or group of microorganisms of interest belongs to thermophilic acidophilic bacteria, the incubation time is preferably at least 20 h, more preferably between 20 h and 30 h, and even more preferably about 24 h, and the temperature is preferably at least 40° C., more preferably between 40° C. and 50° C., and even more preferably about 45° C.

In embodiments wherein the microorganism or group of microorganisms of interest belongs to acetic acid bacteria, the incubation time is preferably at least 35 h, more preferably between 35 h and 72 h, and even more preferably about 48 h, and the temperature is preferably at least 20° C., more preferably between 20° C. and 30° C., and even more preferably about 25° C.

In embodiments wherein the microorganism or group of microorganisms of interest belongs to lactic acid bacteria, the incubation time is preferably at least 20 h, more preferably between 20 h and 48 h, and even more preferably about 24 h, and the temperature is preferably at least 20° C., more preferably between 20° C. and 40° C., and even more preferably about 30° C.

In embodiments wherein the microorganism or group of microorganisms of interest belongs to aerobic mesophilic bacteria, the incubation time is preferably at least 8 h, more preferably between 10 h and 36 h, and even more preferably between 10 h and 24 h, and the temperature is preferably at least 25° C., more preferably between 25° C. and 40° C., and even more preferably about 35° C. However, some mesophilic bacteria such as Methylobacterium extorquens may require longer incubation time, e.g. up to 96 h.

In embodiments wherein the microorganism or group of microorganisms of interest belongs to anaerobic bacteria, the incubation time is preferably at least 10 h, more preferably between 20 h and 72 h, and even more preferably between 20 h and 24 h, and the temperature is preferably at least 20° C., more preferably between 20° C. and 45° C., and even more preferably about 37° C. However, some anaerobic bacteria such as Cutibacterium acnes may require longer incubation time, e.g. up to 96 h.

In embodiments wherein the microorganism or group of microorganisms of interest belongs to heterotrophic bacteria as defined above, the incubation time is preferably at least 8 h, more preferably between 14 h and 48 h, and even more preferably between 24 h and 42 h, and the temperature is preferably between 20° C. and 37° C., more preferably between 32° C. and 37° C., and even more preferably about 32.5° C. However, some heterotrophic bacteria such as Methylobacterium extorquens may require longer incubation time, e.g. up to 96 h at 22.5° C.

In embodiments wherein the microorganism or group of microorganisms of interest belongs to microscopic fungi, the incubation time is preferably at least 20 h, more preferably between 30 h and 50 h, and even more preferably between 30 h and 40 h, and the temperature is preferably at least 20° C., more preferably between 20° C. and 35° C., and even more preferably about 25° C.

Of course, when the group of microorganisms of interest comprises microorganisms belonging to different groups, the incubation time as well as the temperature have to be adjusted in order to allow growth of each microorganism.

In some embodiments, in particular when the method is used to assess sterility of a sample, incubation time may be extended in order to detect growth of any microorganism present in the sample. Typically, in such case, the container may be incubated during at least 48 h, or even at least 72 h.

### Detection

In step c) of the method of the invention, microcolonies formed on the surface of the growth medium, or on the surface of the membrane filter, and emitting a fluorescent signal of at least one fluorescent dye as defined above, are detected and/or enumerated.

Microcolonies emitting a fluorescent signal are detected and/or enumerated using any method known by the skilled person. In particular, microcolonies can be detected and/or enumerated using a fluorescence solid phase cytometer or microscope, preferably using an automated solid phase cytometer or microscope, or any other imaging system suitable to detect fluorescence emission. Automated systems for counting microbial microcolonies emitting a fluorescent signal in culture devices are known in the art. Such automated systems generally comprise an imaging system, an image analysis algorithm to determine a colony count, and a data management system to display and, optionally, store and manipulate the colony count data and images. As example, the microcolony semi-automated counter MICA sold by DIAMIDEX is able to fluorometrically detect and count microorganisms at their early stage of microcolony.

In some embodiments wherein step b) is carried out on a membrane filter, and thus wherein microcolonies are grown on the surface of the membrane filter, said filter may be removed from the solid growth medium after step b) and before step c) and microcolonies thereon are detected and/or enumerated in step c). This step remains optional and microcolonies on the surface of the membrane filter can be detected and/or enumerated directly in the incubation container.

The detection of at least one microcolony indicates the presence of a microorganism of interest in the sample.

Furthermore, it is assumed that each microcolony detected in step c) arises from an individual cell that has undergone cell division. Therefore, by counting the number of microcolonies, and optionally accounting for the dilution or concentration factor, the number of microorganisms of interest in the sample can be determined.

Depending on the use of selective features, such as selective growth medium, selective incubation parameters, etc. The method of the invention may also allow identification of microorganisms in the sample. In particular, the detection of microcolonies may reveal the presence of microorganisms belonging to a specific taxonomic group or functional group (e.g. thermophilic acidophilic bacteria, acetic acid bacteria, lactic acid bacteria, Gram negative bacteria, yeasts or molds, aerobic mesophilic bacteria, anaerobic bacteria etc.).

The method of the invention may comprise additional steps after step c) in order to further characterize microorganisms of the detected microcolonies such as biochemical assays including detection of enzymatic activity or metabolites, assays using immunochemical markers, specific stains, tests or reagents specific for identification and/or detection of specific microorganisms including antibiotics.

### Uses of the Method of the Invention

The present invention also relates to the use of the method of the invention to detect or enumerate any microorganism or group of microorganisms of interest in a sample, in particular to detect or enumerate thermophilic acidophile bacteria (TAB), acetic acid bacteria (AAB), lactic acid bacteria (LAB), anaerobic bacteria, aerobic mesophile bacteria (AMB), yeasts and molds, Gram positive or Gram negative bacteria, heterotrophic bacteria, or any combination thereof, in a sample, preferably to detect or enumerate thermophilic acidophile bacteria (TAB), acetic acid bacteria (AAB), lactic acid bacteria (LAB), anaerobic bacteria, aerobic mesophile bacteria (AMB), yeasts and molds, Gram positive or Gram negative bacteria, or any combination thereof, in a sample.

The present invention also relates to the use of the method of the invention in determining whether a sample contains at least one microorganism of interest, in particular at least one microorganism belonging to thermophilic acidophile bacteria (TAB), acetic acid bacteria (AAB), lactic acid bacteria (LAB), anaerobic bacteria, aerobic mesophile bacteria, yeasts and molds, Gram positive or Gram negative bacteria, heterotrophic bacteria, or any combination thereof, preferably at least one microorganism belonging to thermophilic acidophile bacteria (TAB), acetic acid bacteria (AAB), lactic acid bacteria (LAB), anaerobic bacteria, aerobic mesophile bacteria, yeasts and molds, Gram positive or Gram negative bacteria, or any combination thereof.

All embodiments related to the method of the invention are also contemplated in this aspect.

The present invention also relates to the use of the method of the invention in determining whether a sample is sterile.

As used herein, the term “sterile” refers to the absence in the sample of any microorganism of interest. Depending on the application domain (e.g. pharmaceuticals, cosmetics, foods and beverages, environmental analysis, diagnosis etc.) and standard methods, the microorganisms to be considered in sterility testing may vary. However, typically, the method of the invention is designed in order to favour detection of a maximum of microorganisms that might be contained in the sample.

In this aspect, the method of the invention is used to detect a microorganism or a group of microorganisms of interest, the absence of said microorganism or a group of microorganisms of interest indicating that the sample is sterile, and the detection of said microorganism or a group of microorganisms of interest indicated that the sample is not sterile.

For such application, growth medium is typically non selective medium such as R2A or TSA. Typically, cultures are incubated during at least 72 h at a temperature between 20° C. and 40° C. preferably about 35° C. Growth media and conditions of culture may greatly vary according to the application domain (e.g. pharmaceutical domain, water analysis, etc.) and according to the standard methods recommended in this domain. The skilled person can easily adjust the method of the invention to take into account these parameters.

If necessary, the method of the invention may be repeated to detect different microorganisms or groups of microorganisms, e.g. using different growth media, incubation conditions, fluorescent dye(s) etc.

Preferably, for such application, the sample is contacted with at least one fluorescent dye selected from the group consisting of xanthene-based dyes, preferably selected from the group consisting of rhodamine dyes, fluorescein dyes and carbopyronine-based dyes, coumarin-based dyes, cyanine-based dyes, pyrene-based dyes, squaraine-based dyes and squaraine rotaxane-based dyes, and combinations thereof, preferably with at least one fluorescent dye selected from the group consisting of Alexa Fluor™ 350, Pacific Blue™, Cy5™, preferably Sulfo-Cy5, Alexa Fluor™ 405, Cascade Blue™, Seta dyes, preferably Seta 650 or Seta 375, SeTau dyes, preferably SeTau 647 or SeTau 488, Alexa Fluor 488 and MB660R, and derivatives and combinations thereof, and even more preferably with at least one fluorescent dye selected from the group consisting of Alexa Fluor™ 350-NHS ester, Pacific Blue™-NHS ester, Sulfo-Cy5-acid, Alexa Fluor™ 405-DBCO, Cascade Blue™, Seta 650-DBCO, Seta 375-NHS, SeTau 647-Maleimide, SeTau 488-NHS, Alexa Fluor 488-acid, MB660R-acid and MB660R-DBCO, and combinations thereof. In particular, said at least one fluorescent dye may be selected from the group consisting of Alexa Fluor™ 350, Pacific Blue™, Cy5™, preferably Sulfo-Cy5, Alexa Fluor™ 405, Cascade Blue™, Seta dyes, preferably Seta 650, SeTau dyes, preferably SeTau 647 or SeTau 488, Alexa Fluor 488 and MB660R, and derivatives and combinations thereof, preferably selected from the group consisting of Alexa Fluor™ 350-NHS ester, Pacific Blue™-NHS ester, Sulfo-Cy5-acid, Alexa Fluor™ 405-DBCO, Cascade Blue™, Seta 650-DBCO, SeTau 647-Maleimide, SeTau 488-NHS, Alexa Fluor 488-acid, MB660R-acid and MB660R-DBCO, and combinations thereof.

In particular, said at least one fluorescent dye may be selected from the group consisting of Alexa Fluor™ 350, Cy5™, preferably Sulfo-Cy5, Alexa Fluor™ 405, Seta dyes, preferably Seta 650, SeTau dyes, preferably SeTau 647, Alexa Fluor 488 and MB660R, and derivatives and combinations thereof, and preferably selected from the group consisting of Alexa Fluor™ 350-NHS ester, Sulfo-Cy5-acid, Alexa Fluor™ 405-DBCO, Seta 650-DBCO, SeTau 647-Maleimide, Alexa Fluor 488-acid, MB660R-acid and MB660R-DBCO, and combinations thereof.

More particularly, said at least one fluorescent dye may be a combination of xanthene-based dyes and cyanine-based dyes, preferably a combination of Cy5™, preferably Sulfo-Cy5, or a derivative thereof, and MB™ 660R or a derivative thereof, more preferably a combination of MB™ 660R DBCO and Sulfo-Cy5-acid.

In some embodiments, said at least one fluorescent dye is a combination selected from the group consisting of Acridine orange—DAPI, Acridine orange—Seta 375-NHS, Acridine orange—MB660R acid, Acridine orange—Alexa Fluor 350-NHS Ester, Acridine orange—Pacific Blue-NHS Ester, Acridine orange—Sulfo Cy5-acid, Acridine orange—BODIPY, Acridine orange—Prodan, Acridine orange—Alexa Fluor 405-DBCO, Acridine orange—Cascade blue, Acridine orange—Alexa Fluor 488-Acid, DRAQ5—Alexa Fluor 350-NHS Ester, DRAQ5—Pacific Blue-NHS Ester, DRAQ5—Sulfo Cy5-acid, DRAQ5—BODIPY 500/510, DRAQ5—Prodan, DRAQ5—Alexa Fluor 405-DBCO, DRAQ5—Cascade blue, DRAQ5—Seta 650-DBCO, DRAQ5—SeTau 647-Maleimide, DRAQ5—MB660R-Acid, DRAQ5—MB660R-DBCO, DRAQ5—ATTO647-Acid, Cytrak orange—Alexa Fluor 350-NHS Ester, Cytrak orange—Pacific Blue-NHS Ester, Cytrak orange—Sulfo Cy5-acid, Cytrak orange—BODIPY 500/510, Cytrak orange—Prodan, Cytrak orange—Alexa Fluor 405-DBCO, Cytrak orange—Cascade blue, Seta 375-NHS—Alexa Fluor 350-NHS Ester, Seta 375-NHS—Pacific Blue-NHS Ester, Seta 375-NHS—Sulfo Cy5-acid, Seta 375-NHS—BODIPY 500/510, Seta 375-NHS—Prodan, Seta 375-NHS—Alexa Fluor 405-DBCO, Seta 375-NHS—Cascade blue, Alexa Fluor 488-Acid—Alexa Fluor 350-NHS Ester, Alexa Fluor 488-Acid—Pacific Blue-NHS Ester, Alexa Fluor 488-Acid—Sulfo Cy5-acid, Alexa Fluor 488-Acid—BODIPY 500/510, Alexa Fluor 488-Acid—Prodan, Alexa Fluor 488-Acid—Alexa Fluor 405-DBCO, Alexa Fluor 488-Acid—Cascade blue, MB660R-Acid—Alexa Fluor 350-NHS Ester, MB660R-Acid—Pacific Blue-NHS Ester, MB660R-Acid—Sulfo Cy5-acid, MB660R-Acid—BODIPY 500/510, MB660R-Acid—Prodan, MB660R-Acid—Alexa Fluor 405-DBCO, MB660R-Acid—Cascade blue, MB660R-Acid—Seta 650-DBCO, MB660R-Acid—SeTau 647-Maleimide, MB660R-DBCO—Alexa Fluor 350-NHS Ester, MB660R-DBCO—Pacific Blue-NHS Ester, MB660R-DBCO—Sulfo Cy5-acid, MB660R-DBCO—BODIPY 500/510, MB660R-DBCO—Prodan, MB660R-DBCO—Alexa Fluor 405-DBCO, MB660R-DBCO—Cascade blue, MB660R-DBCO—Seta 650-DBCO, MB660R-DBCO—SeTau 647-Maleimide, ATTO647-Acid—Sulfo Cy5-acid, ATTO647-Acid—Seta 650-DBCO, ATTO647-Acid—SeTau 647-Maleimide, ATTO647-Acid—MB660R-Acid, ATTO647-Acid—MB660R-DBCO, Sulfo Cy5.5-acid—Sulfo Cy5-acid, Sulfo Cy5-acid—Seta 650-DBCO, Sulfo Cy5-acid—SeTau 647-Maleimide and Seta 650-DBCO—SeTau 647-Maleimide, preferably selected from the group consisting of Acridine orange—Seta 375-NHS, Acridine orange—MB660R acid, Acridine orange—Alexa Fluor 350-NHS Ester, Acridine orange—Pacific Blue-NHS Ester, Acridine orange—Sulfo Cy5-acid, Acridine orange—BODIPY, Acridine orange—Prodan, Acridine orange—Alexa Fluor 405-DBCO, Acridine orange—Cascade blue, Acridine orange—Alexa Fluor 488-Acid, DRAQ5—Alexa Fluor 350-NHS Ester, DRAQ5—Pacific Blue-NHS Ester, DRAQ5—Sulfo Cy5-acid, DRAQ5—BODIPY 500/510, DRAQ5—Prodan, DRAQ5—Alexa Fluor 405-DBCO, DRAQ5—Cascade blue, DRAQ5—Seta 650-DBCO, DRAQ5—SeTau 647-Maleimide, DRAQ5—MB660R-Acid, DRAQ5—MB660R-DBCO, DRAQ5—ATTO647-Acid, Cytrak orange—Alexa Fluor 350-NHS Ester, Cytrak orange—Pacific Blue-NHS Ester, Cytrak orange—Sulfo Cy5-acid, Cytrak orange—BODIPY 500/510, Cytrak orange—Prodan, Cytrak orange—Alexa Fluor 405-DBCO, Cytrak orange—Cascade blue, Seta 375-NHS—Alexa Fluor 350-NHS Ester, Seta 375-NHS—Pacific Blue-NHS Ester, Seta 375-NHS—Sulfo Cy5-acid, Seta 375-NHS—BODIPY 500/510, Seta 375-NHS—Prodan, Seta 375-NHS—Alexa Fluor 405-DBCO, Seta 375-NHS—Cascade blue, Alexa Fluor 488-Acid—Alexa Fluor 350-NHS Ester, Alexa Fluor 488-Acid—Pacific Blue-NHS Ester, Alexa Fluor 488-Acid—Sulfo Cy5-acid, Alexa Fluor 488-Acid—BODIPY 500/510, Alexa Fluor 488-Acid—Prodan, Alexa Fluor 488-Acid—Alexa Fluor 405-DBCO, Alexa Fluor 488-Acid—Cascade blue, MB660R-Acid—Alexa Fluor 350-NHS Ester, MB660R-Acid—Pacific Blue-NHS Ester, MB660R-Acid—Sulfo Cy5-acid, MB660R-Acid—BODIPY 500/510, MB660R-Acid—Prodan, MB660R-Acid—Alexa Fluor 405-DBCO, MB660R-Acid—Cascade blue, MB660R-Acid—Seta 650-DBCO, MB660R-Acid—SeTau 647-Maleimide, MB660R-DBCO—Alexa Fluor 350-NHS Ester, MB660R-DBCO—Pacific Blue-NHS Ester, MB660R-DBCO—Sulfo Cy5-acid, MB660R-DBCO—BODIPY 500/510, MB660R-DBCO—Prodan, MB660R-DBCO—Alexa Fluor 405-DBCO, MB660R-DBCO—Cascade blue, MB660R-DBCO—Seta 650-DBCO, MB660R-DBCO—SeTau 647-Maleimide, ATTO647-Acid—Sulfo Cy5-acid, ATTO647-Acid—Seta 650-DBCO, ATTO647-Acid—SeTau 647-Maleimide, ATTO647-Acid—MB660R-Acid, ATTO647-Acid—MB660R-DBCO, Sulfo Cy5.5-acid—Sulfo Cy5-acid, Sulfo Cy5-acid—Seta 650-DBCO, Sulfo Cy5-acid—SeTau 647-Maleimide and Seta 650-DBCO—SeTau 647-Maleimide.

All embodiments related to the method of the invention are also contemplated in this aspect.

The present invention also relates to the use of the method of the invention in determining the bioburden of a sample.

As used herein, the term “bioburden” refers to the number of living microorganisms of interest present in a sample. Depending on the application domain (e.g. pharmaceuticals, cosmetics, foods and beverages, environmental analysis, diagnosis etc.) and standard methods, the microorganisms to be considered in bioburden testing may vary. However, typically, the method of the invention is designed in order to favour detection of a maximum of microorganisms that might be contained in the sample.

In this aspect, the method of the invention is used to detect and enumerate a microorganism or a group of microorganisms of interest, enumeration of said microorganism or a group of microorganisms of interest is used to evaluate the bioburden of the sample.

For such application, growth medium is typically non selective medium such as R2A, TSA or SDA agar. Typically, cultures are incubated during at least 30 h at a temperature between 20° C. and 40° C. preferably about 35° C. Growth media and conditions of culture may greatly vary according to the application domain (e.g. pharmaceutical domain, water analysis, etc.) and according to the standard methods recommended in this domain. The skilled person can easily adjust the method of the invention to take into account these parameters.

If necessary, the method of the invention may be repeated to detect and enumerate different microorganisms or groups of microorganisms, e.g. using different growth media, incubation conditions, fluorescent dye(s) etc.

Preferably, for such application, the sample is contacted with at least one fluorescent dye selected from the group consisting of xanthene-based dyes, preferably selected from the group consisting of rhodamine dyes, fluorescein dyes and carbopyronine-based dyes, coumarin-based dyes, cyanine-based dyes, pyrene-based dyes, squaraine-based dyes and squaraine rotaxane-based dyes, and combinations thereof, more preferably with at least one fluorescent dye selected from the group consisting of Alexa Fluor™ 350, Pacific Blue™, Cy5™, preferably Sulfo-Cy5, Alexa Fluor™ 405, Cascade Blue™, Seta dyes, preferably Seta 650 or Seta 375, SeTau dyes, preferably SeTau 647 or SeTau 488, Alexa Fluor 488 and MB660R, and derivatives and combinations thereof, and even more preferably with at least one fluorescent dye selected from the group consisting of Alexa Fluor™ 350-NHS ester, Pacific Blue™-NHS ester, Sulfo-Cy5-acid, Alexa Fluor™ 405-DBCO, Cascade Blue™, Seta 650-DBCO, Seta 375-NHS, SeTau 647-Maleimide, SeTau 488-NHS, Alexa Fluor 488-acid, MB660R-acid and MB660R-DBCO, and combinations thereof. More particularly, said at least one fluorescent dye may be selected from the group consisting of Alexa Fluor™ 350, Pacific Blue™, Cy5™, preferably Sulfo-Cy5, Alexa Fluor™ 405, Cascade Blue™, Seta dyes, preferably Seta 650, SeTau dyes, preferably SeTau 647 or SeTau 488, Alexa Fluor 488 and MB660R, and derivatives and combinations thereof, and even more preferably with at least one fluorescent dye selected from the group consisting of Alexa Fluor™ 350-NHS ester, Pacific Blue™-NHS ester, Sulfo-Cy5-acid, Alexa Fluor™ 405-DBCO, Cascade Blue™, Seta 650-DBCO, SeTau 647-Maleimide, SeTau 488-NHS, Alexa Fluor 488-acid, MB660R-acid and MB660R-DBCO, and combinations thereof.

In particular, said at least one fluorescent dye may be selected from the group consisting of Alexa Fluor™ 350, Cy5™, preferably Sulfo-Cy5, Alexa Fluor™ 405, Seta dyes, preferably Seta 650, SeTau dyes, preferably SeTau 647, Alexa Fluor 488 and MB660R, and derivatives and combinations thereof, and preferably selected from the group consisting of Alexa Fluor™ 350-NHS ester, Sulfo-Cy5-acid, Alexa Fluor™ 405-DBCO, Seta 650-DBCO, SeTau 647-Maleimide, Alexa Fluor 488-acid, MB660R-acid and MB660R-DBCO, and combinations thereof. More particularly, said at least one fluorescent dye may be a combination of xanthene-based dyes and cyanine-based dyes, preferably a combination of Cy5™, preferably Sulfo-Cy5, or a derivative thereof, and MB™ 660R or a derivative thereof, more preferably a combination of MB™ 660R DBCO and Sulfo-Cy5-acid. Optionally, one or several additional fluorescent dyes can be added to said at least one fluorescent dye as defined above, in particular one or several additional fluorescent dyes selected from the group consisting of anthracene-based dyes, dipyrromethene-based dyes and naphthalene-based dyes, and combinations thereof, preferably selected from the group consisting of DRAQ5™, BODIPY™ dyes, preferably BODIPY 500/510 and prodan, and derivatives and combinations thereof, more preferably selected from the group consisting of DRAQ5™, BODIPY 500/510 and prodan, and combinations thereof.

In some embodiments, said at least one fluorescent dye is a combination selected from the group consisting of Acridine orange—DAPI, Acridine orange—Seta 375-NHS, Acridine orange—MB660R acid, Acridine orange—Alexa Fluor 350-NHS Ester, Acridine orange—Pacific Blue-NHS Ester, Acridine orange—Sulfo Cy5-acid, Acridine orange—BODIPY, Acridine orange—Prodan, Acridine orange—Alexa Fluor 405-DBCO, Acridine orange—Cascade blue, Acridine orange—Alexa Fluor 488-Acid, DRAQ5—Alexa Fluor 350-NHS Ester, DRAQ5—Pacific Blue-NHS Ester, DRAQ5—Sulfo Cy5-acid, DRAQ5—BODIPY 500/510, DRAQ5—Prodan, DRAQ5—Alexa Fluor 405-DBCO, DRAQ5—Cascade blue, DRAQ5—Seta 650-DBCO, DRAQ5—SeTau 647-Maleimide, DRAQ5—MB660R-Acid, DRAQ5—MB660R-DBCO, DRAQ5—ATTO647-Acid, Cytrak orange—Alexa Fluor 350-NHS Ester, Cytrak orange—Pacific Blue-NHS Ester, Cytrak orange—Sulfo Cy5-acid, Cytrak orange—BODIPY 500/510, Cytrak orange—Prodan, Cytrak orange—Alexa Fluor 405-DBCO, Cytrak orange—Cascade blue, Seta 375-NHS—Alexa Fluor 350-NHS Ester, Seta 375-NHS—Pacific Blue-NHS Ester, Seta 375-NHS—Sulfo Cy5-acid, Seta 375-NHS—BODIPY 500/510, Seta 375-NHS—Prodan, Seta 375-NHS—Alexa Fluor 405-DBCO, Seta 375-NHS—Cascade blue, Alexa Fluor 488-Acid—Alexa Fluor 350-NHS Ester, Alexa Fluor 488-Acid—Pacific Blue-NHS Ester, Alexa Fluor 488-Acid—Sulfo Cy5-acid, Alexa Fluor 488-Acid—BODIPY 500/510, Alexa Fluor 488-Acid—Prodan, Alexa Fluor 488-Acid—Alexa Fluor 405-DBCO, Alexa Fluor 488-Acid—Cascade blue, MB660R-Acid—Alexa Fluor 350-NHS Ester, MB660R-Acid—Pacific Blue-NHS Ester, MB660R-Acid—Sulfo Cy5-acid, MB660R-Acid—BODIPY 500/510, MB660R-Acid—Prodan, MB660R-Acid—Alexa Fluor 405-DBCO, MB660R-Acid—Cascade blue, MB660R-Acid—Seta 650-DBCO, MB660R-Acid—SeTau 647-Maleimide, MB660R-DBCO—Alexa Fluor 350-NHS Ester, MB660R-DBCO—Pacific Blue-NHS Ester, MB660R-DBCO—Sulfo Cy5-acid, MB660R-DBCO—BODIPY 500/510, MB660R-DBCO—Prodan, MB660R-DBCO—Alexa Fluor 405-DBCO, MB660R-DBCO—Cascade blue, MB660R-DBCO—Seta 650-DBCO, MB660R-DBCO—SeTau 647-Maleimide, ATTO647-Acid—Sulfo Cy5-acid, ATTO647-Acid—Seta 650-DBCO, ATTO647-Acid—SeTau 647-Maleimide, ATTO647-Acid—MB660R-Acid, ATTO647-Acid—MB660R-DBCO, Sulfo Cy5.5-acid—Sulfo Cy5-acid, Sulfo Cy5-acid—Seta 650-DBCO, Sulfo Cy5-acid—SeTau 647-Maleimide and Seta 650-DBCO—SeTau 647-Maleimide, preferably selected from the group consisting of Acridine orange—Seta 375-NHS, Acridine orange—MB660R acid, Acridine orange—Alexa Fluor 350-NHS Ester, Acridine orange—Pacific Blue-NHS Ester, Acridine orange—Sulfo Cy5-acid, Acridine orange—BODIPY, Acridine orange—Prodan, Acridine orange—Alexa Fluor 405-DBCO, Acridine orange—Cascade blue, Acridine orange—Alexa Fluor 488-Acid, DRAQ5—Alexa Fluor 350-NHS Ester, DRAQ5—Pacific Blue-NHS Ester, DRAQ5—Sulfo Cy5-acid, DRAQ5—BODIPY 500/510, DRAQ5—Prodan, DRAQ5—Alexa Fluor 405-DBCO, DRAQ5—Cascade blue, DRAQ5—Seta 650-DBCO, DRAQ5—SeTau 647-Maleimide, DRAQ5—MB660R-Acid, DRAQ5—MB660R-DBCO, DRAQ5—ATTO647-Acid, Cytrak orange—Alexa Fluor 350-NHS Ester, Cytrak orange—Pacific Blue-NHS Ester, Cytrak orange—Sulfo Cy5-acid, Cytrak orange—BODIPY 500/510, Cytrak orange—Prodan, Cytrak orange—Alexa Fluor 405-DBCO, Cytrak orange—Cascade blue, Seta 375-NHS—Alexa Fluor 350-NHS Ester, Seta 375-NHS—Pacific Blue-NHS Ester, Seta 375-NHS—Sulfo Cy5-acid, Seta 375-NHS—BODIPY 500/510, Seta 375-NHS—Prodan, Seta 375-NHS—Alexa Fluor 405-DBCO, Seta 375-NHS—Cascade blue, Alexa Fluor 488-Acid—Alexa Fluor 350-NHS Ester, Alexa Fluor 488-Acid—Pacific Blue-NHS Ester, Alexa Fluor 488-Acid—Sulfo Cy5-acid, Alexa Fluor 488-Acid—BODIPY 500/510, Alexa Fluor 488-Acid—Prodan, Alexa Fluor 488-Acid—Alexa Fluor 405-DBCO, Alexa Fluor 488-Acid—Cascade blue, MB660R-Acid—Alexa Fluor 350-NHS Ester, MB660R-Acid—Pacific Blue-NHS Ester, MB660R-Acid—Sulfo Cy5-acid, MB660R-Acid—BODIPY 500/510, MB660R-Acid—Prodan, MB660R-Acid—Alexa Fluor 405-DBCO, MB660R-Acid—Cascade blue, MB660R-Acid—Seta 650-DBCO, MB660R-Acid—SeTau 647-Maleimide, MB660R-DBCO—Alexa Fluor 350-NHS Ester, MB660R-DBCO—Pacific Blue-NHS Ester, MB660R-DBCO—Sulfo Cy5-acid, MB660R-DBCO—BODIPY 500/510, MB660R-DBCO—Prodan, MB660R-DBCO—Alexa Fluor 405-DBCO, MB660R-DBCO—Cascade blue, MB660R-DBCO—Seta 650-DBCO, MB660R-DBCO—SeTau 647-Maleimide, ATTO647-Acid—Sulfo Cy5-acid, ATTO647-Acid—Seta 650-DBCO, ATTO647-Acid—SeTau 647-Maleimide, ATTO647-Acid—MB660R-Acid, ATTO647-Acid—MB660R-DBCO, Sulfo Cy5.5-acid—Sulfo Cy5-acid, Sulfo Cy5-acid—Seta 650-DBCO, Sulfo Cy5-acid—SeTau 647-Maleimide and Seta 650-DBCO—SeTau 647-Maleimide.

All embodiments related to the method of the invention are also contemplated in this aspect.

### Kit and Uses Thereof

The present invention further relates to a kit comprising
at least one fluorescent dye, as defined above;
and optionally
at least one membrane filter as defined above.

The kit may further comprise
at least one container comprising a solid growth medium containing nutrients to support growth of a microorganism or a group of microorganisms of interest, or materials needed to obtained said at least one container and/or
a leaflet providing instructions to use said kit.

In a particular embodiment, the kit of the invention comprises
at least one fluorescent dye, as defined above; and
at least one membrane filter as defined above;
and optionally at least one container comprising a solid growth medium containing nutrients to support growth of a microorganism or a group of microorganisms of interest, or materials needed to obtained said at least one container and/or a leaflet providing instructions to use said kit.

In another particular embodiment, the kit of the invention comprises
at least one container comprising a solid growth medium containing at least one fluorescent dye, preferably as defined above, and nutrients to support growth of the microorganism or a group of microorganisms of interest, or materials needed to obtained said at least one container, and
and optionally
at least one membrane filter, preferably as defined above, and/or
a leaflet providing instructions to use said kit.

All embodiments related to the method of the invention are also contemplated in this aspect.

The present invention also relates to the use of a kit according to the invention to detect or enumerate a microorganism or a group of microorganisms of interest according to the method of the invention as described above. All embodiments related to the method of the invention are also contemplated in this aspect.

All the references cited in this description are incorporated by reference in the present application. Others features and advantages of the invention will become clearer in the following examples which are given for purposes of illustration and not by way of limitation.

### EXAMPLES

Excepted for Aspergillus brasiliensis and Cutibacterium acnes, all strains were diluted in NaCl 0.9% from cryopreserved stocks previously quantified to obtain a defined amount of microorganisms per experimental condition and each bacterial preparation was filtered on 0.451 μm PVDF or MCE membrane (47 mm).

For A. brasiliensis and C. acnes, BioBalls® from Biomerieux containing a precise number of microorganisms (BioBall® Multishot 550 Aspergillus Brasiliensis SKU number: 56001; BioBall® Multishot Propionibacterium acnes (former name of Cutibacterium acnes, strain DSM 1897) SKU Number: 416600) were diluted in NaCl 0.9% to obtain a defined amount of microorganisms per experimental condition and each bacterial preparation was filtered on 0.45 μm PVDF or MCE membrane (47 mm).

### Example 1: Detection of Thermophilic Acidophilic Bacteria (TAB)

Several strains of Alicyclobacillus bacteria including Alicyclobacillus acidoterrestris ATCC® 49025™, Alicyclobacillus acidocaldarius ATCC® 27009™, Alicyclobacillus acidiphilus DSM 14558, Alicyclobacillus cycloheptanicus ATCC® 49028™, Alicyclobacillus hesperidum DSM 12766, Alicyclobacillus herbarius DSM 13609 and Alicyclobacillus contaminans DSM 17975 were used in this example.

150 μL of a fluorescent dye selected from the group consisting of Alexa Fluor 488-acid (50 μM), Alexa Fluor 488-DBCO (50 μM), MB660R-acid (250 μM), MB660R-DBCO (250 μM) and ATTO647-acid (5 μM), were deposited BAT agar plates (Yeast extract 2 g/l, D(+) glucose 5 g/l, Calcium chloride 0.25066 g/l, Magnesium sulfate 0.5 g/l, Ammonium sulfate 0.2 g/l, Potassium dihydrogen phosphate 3 g/l, Zinc sulfate 0.00018 g/l, Copper sulfate 0.00016 g/l, Manganese sulfate 0.00015 g/l, Sodium molybdate dihydrate 0.0003 g/l, Agar-Agar 18 g/l, pH value 3.8-4.2). Each agar plate comprised one drop of a fluorescence dye.

A filtration membrane was then deposited above the fluorescent dye drop on each agar plate. Plates were then incubated 24 h at 45° C. except for A. acidocaldarius and A. contaminans that were incubated 48 h at 45° C.

After incubation, microcolonies were detected using a microcolony semi-automated counter (MICA, DIAMIDEX, Light power 100%, Gain 1, Exposure time 100 and 400 ms).

Tested conditions and results are presented in Table 2.

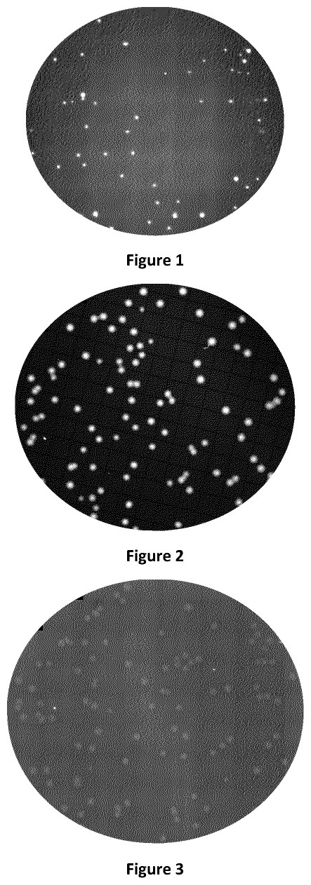

TABLE 2
Labelling and detection of TAB strains

| | | | Labelling and |
|---|---|---|---|
| Fluorescent dye | Membrane | Bacterial strains | detection |
| Alexa Fluor | PVDF | A. acidoterrestris | + |
| 488-Acid | | A. acidiphilus | + |
| Alexa Fluor | PVDF | A. acidoterrestris | + |
| 488-DBCO | | A. acidiphilus | + |
| MB660R-Acid | PVDF | A. acidoterrestris | + |
| | | A. acidiphilus | + |
| | | A. cycloheptanicus | + |
| | | A. herbarius | + |
| | | A. hesperidum | + |
| | | A. contaminans | + |
| MB660R-DBCO | PVDF | A. acidoterrestris | + (FIG. 1) |
| | | A. acidocaldarius | + |
| | | A. acidiphilus | + |
| | | A. contaminans | + |
| | | A. herbarius | + |
| | | A. hesperidum | + |
| | | A. cycloheptanicus | + |
| | MCE | A. acidiphilus | + |
| | | A. acidocaldarius | + |
| ATTO647-Acid | PVDF | A. acidoterrestris | + |
| | | A. acidiphilus | + |

### Example 2: Detection of Acetic Acid Bacteria (AAB)

Acetic acid bacterial strains used in this example are Acetobacter aceti ATCC® 15973™ Gluconoacetobacter liquefaciens ATCC® 14835™; Asaia siamensis DSM 15972 and Gluconobacter oxydans ATCC® 19357™.

150 μL of a fluorescent dye selected from the group consisting of DAPI (50 μM), Alexa Fluor 488-acid (50 μM), Alexa Fluor 488-DBCO (50 μM), MB660R-acid (250 μM), MB660R-DBCO (250 μM) and ATTO647-acid (5 μM), were deposited YM agar plates (Glucose 10 g/L, Malt extract 3 g/L, Peptone 5 g/L, Yeast extract 3 g/L, Agar 15 g/L, pH value 6.2±0.2). Each agar plate comprised one drop of a fluorescence dye.

A filtration membrane was then deposited above the fluorescent dye drop on each agar plate. Plates were then incubated 72 h at 25° C.

After incubation, microcolonies were detected using a microcolony semi-automated counter (MICA, DIAMIDEX, Cy5 Light power 100%, Gain 1, Exposure time 200 ms for MB660R-DBCO and ATTO647-Acid, 300 ms for AF488-Acid, 400 ms for MB660R-Acid, 600 ms for AF488-DBCO, UV Light power 12%, Gain 1, Exposure time 400 ms for DAPI).

Tested conditions and results are presented in Table 3.

TABLE 3
Labelling and detection of AAB strains

| | | | Labelling and |
|---|---|---|---|
| Fluorescent dye | Membrane | Bacterial strains | detection |
| DAPI | MCE | A. aceti | + |
| | | G. oxydans | + |
| | | G. liquefaciens | + |
| Alexa Fluor | PVDF | A. aceti | + |
| 488-Acid | | G. oxydans | + |
| | | G. liquefaciens | + |
| | | A. siamensis | + |
| Alexa Fluor | PVDF | A. aceti | + |
| 488-DBCO | | G. liquefaciens | + |
| | | A. siamensis | + |
| MB660R-Acid | PVDF | A. aceti | + |
| | | G. oxydans | + |
| | | G. liquefaciens | + |
| | | A. siamensis | + |
| MB660R-DBCO | PVDF | A. aceti | + |
| | | G. oxydans | + |
| | | G. liquefaciens | + |
| ATTO647-Acid | PVDF | A. aceti | + |
| | | G. oxydans | + |
| | | G. liquefaciens | + |
| | | A. siamensis | + |

### Example 3: Detection of Lactic Acid Bacteria (LAB)

Lactic acid bacterial strains used in this example are Lactobacillus plantarum ATCC® 8014™, Weissella confusa ATCC® 10881™ and Lactobacillus casei ATCC® 393™.

150 μL of a fluorescent dye selected from the group consisting of DAPI (50 μM), Alexa Fluor 488-acid (50 μM), Alexa Fluor 488-DBCO (50 μM), MB660R-acid (250 μM), MB660R-DBCO (250 μM) and ATTO647-acid (5 μM), were deposited MRS agar plates (Diammonium hydrogen citrate 2 g/L, Dipotassium hydrogen phosphate 2 g/L, D(+)-glucose 20 g/L, Magnesium sulfate 0.1 g/L, Manganous sulfate 0.05 g/L, Meat extract 5 g/L, Sodium acetate 5 g/L, Universal peptone 10 g/L, Yeast extract 5 g/L, Agar 12 g/L, pH value 5.7). Each agar plate comprised one drop of a fluorescence dye.

A filtration membrane was then deposited above the fluorescent dye drop on each agar plate. Plates were then incubated 48 h at 30° C.

After incubation, microcolonies were detected using a microcolony semi-automated counter (MICA, DIAMIDEX, Cy5 Light power 100%, Gain 1, Exposure time 200 ms for MB660R-DBCO and ATTO647-Acid, 300 ms for AF488-Acid, 400 ms for MB660R-Acid, 600 ms for AF488-DBCO, UV Light power 12%, Gain 1, Exposure time 100 ms for DAPI excepted for L. casei done with an UV light power of 35%).

Tested conditions and results are presented in Table 4.

TABLE 4
Labelling and detection of LAB strains

| | | | Labelling and |
|---|---|---|---|
| Fluorescent dye | Membrane | Bacterial strains | detection |
| DAPI | MCE | L. plantarum | + |
| | | W. confusa | + |
| | | L. casei | + |
| Alexa Fluor | PVDF | L. casei | + |
| 488-Acid | | L. plantarum | + |
| | | W. confusa | + |
| Alexa Fluor | PVDF | L. casei | + |
| 488-DBCO | | L. plantarum | + |
| MB660R-Acid | PVDF | L. casei | + |
| | | L. plantarum | + |
| | | W. confusa | + |
| MB660R-DBCO | PVDF | L. plantarum | + |
| | | W. confusa | + |
| ATTO647-Acid | PVDF | L. plantarum | + |
| | | W. confusa | + |

### Example 4: Detection of Anaerobic Bacteria

Anaerobic bacterial strains used in this example are Clostridium sporogenes ATCC 19404 and Cutibacterium acnes ATCC 6919.

200 μL of DAPI (50 μM) were deposited on TSA agar plates (Tryptone 15 g/L, Soja papainic peptone 5 g/L, Sodium chloride 5 g/L, Agar 15 g/L, pH value 7.3±0.2), 150 μl of a mix of Sulfo-Cy5-acid (100 μM) and MB660R-DBCO (100 μM) were deposited on TSA agar plates for C. sporogenes and on Columbia agar plates supplemented with 5% sheep blood (pancreatic digest of casein 12 g/L, peptic digest of animal tissue 5 g/L, yeast extract 3 g/L, beef extract 3 g/L, corn starch 1 g/L, sodium chloride 5 g/L, agar 13.5 g/L, defibrinated sheep blood 5%, pH value pH 7.3±0.2) for C. acnes. Each agar plate comprised one drop of a fluorescence dye.

A filtration membrane was then deposited above the fluorescent dye drop on each agar plate. Plates comprising Clostridium sporogenes were then incubated between 15 h for DAPI staining and 24 h for MB660R-DBCO and Sulfo-Cy5-Acid staining at 35° C. in anaerobic conditions. Plates comprising Cutibacterium acnes were incubated 96 h at 35° C. in anaerobic conditions.

After incubation, microcolonies were detected using a microcolony semi-automated counter (MICA, DIAMIDEX, Cy5 Light power 100%, Gain 1, Exposure time 200 ms for the mix of Sulfo-Cy5-acid and MB660R-DBCO, UV Light power 12%, Gain 1, Exposure time 400 ms for DAPI).

Tested conditions and results are presented in Table 5.

TABLE 5
Labelling and detection of anaerobic bacterial strains

| | | | Labelling and |
|---|---|---|---|
| Fluorescent dye | Membrane | Bacterial strains | detection |
| MB660R-DBCO and | PVDF | C. sporogenes | + |
| Sulfo-CY5-acid | | C. acnes | + |
| DAPI | MCE | C. sporogenes | + |

### Example 5: Detection of Aerobic Mesophilic Bacteria

Mesophile bacterial strains used in this example are Pseudomonas aeruginosa ATCC® 9027, Bacillus subtilis ATCC® 6633™, Escherichia coli ATCC® 8739™, Staphylococcus aureus ATCC® 6538™, Acinetobacter baumannii ATCC 19606, Cronobacter sakazakii ATCC® 29544™, Klebsiella pneumoniae ATCC®13883™, Salmonella typhimurium ATCC®13311™, Enterococcus faecalis ATCC™ 29212™, Shigella sonnei ATCC® 25931™, Kocuria rhizophila ATCC® 9341™, Methylobacterium extorquens ATCC 43645 and Burkholderia cepacia ATCC® 25608™.

150 μL of a fluorescent dye selected from the group consisting of Acridine Orange (40 μM), Cytrak Orange (50 μM), DRAQ5 (125 μM), AF350-NHS Ester (50 μM), Pacific Blue-NHS Ester (10 μM), Sulfo-Cy5-acid (150 μM), Sulfo-Cy5.5 acid (50 μM), BODIPY 500/510 (10 μM on PVDF membrane and 150 μM on MCE membrane), Seta 375-NHS (5 μM), Prodan (15 μM on PVDF membrane and 50 μM on MCE membrane), AF405-DBCO (50 μM), Cascade Blue (1 μM), Seta 650-DBCO (100 μM), SeTau 488-NHS (50 μM), SeTau 647-Maleimide (100 μM), DAPI (50 μM), Alexa Fluor 488-acid (50 μM), Alexa Fluor 488-DBCO (50 μM), MB660R-acid (250 μM), MB660R-DBCO (250 μM) and ATTO647-acid (5 μM), were deposited on TSA agar plates and PCA agar plates (Tryptone 5 g/L, Yeast extract 2.5 g/L, Dextrose 1 g/L, Agar 15 g/L, pH value 7.0±0.2) for Bacillus subtilis, Acinetobacter baumannii, Cronobacter sakazakii, Klebsiella pneumoniae, Salmonella typhimurium, Enterococcus faecalis, Shigella sonnei and Kocuria rhizophila or on TSA agar plates for Methylobacterium extorquens, Pseudomonas aeruginosa, Escherichia coli, Staphylococcus aureus and Burkholderia cepacia. Each agar plate comprised one drop of a fluorescence dye.

A filtration membrane was then deposited above the fluorescent dye drop on each agar plate. Plates were then incubated at 35° C., 11 h for Cronobacter sakazakii on PCA and TSA plates and for Klebsiella pneumoniae on TSA plates, 18 h for Klebsiella pneumoniae on PCA plates, for Bacillus subtilis, Acinetobacter baumannii, Salmonella typhimurium, Enterococcus faecalis, Shigella sonnei, Kocuria rhizophila on PCA and TSA plates and for Pseudomonas aeruginosa on TSA plates, 24 h for Burkholderia cepacia on TSA plates and 96 h for Methylobacterium extorquens on TSA plates.

After incubation, microcolonies were detected using a microcolony semi-automated counter (MICA, DIAMIDEX, DAPI: UV light power 12% Gain 1 Exposure time 200 ms; MB660R-DBCO, MB660R-acid, ATTO647-acid: CY5 light power 100% Gain 1 Exposure time 200 and 500 ms; AF488-Acid and AF488-DBCO: FITC light power 100% Gain 1 Exposure time 200 and 400 ms; Sulfo-CY5-Acid, Seta-650 DBCO: CY5 light power 100% Gain 1 Exposure time 50 ms and 400 ms; AF350-NHS Ester, AF405-DBCO: UV light power: 20% or 50% Gain 1 Exposure time 100 ms and 400 ms; SeTau 647-Maleimide: CY5 light power 100% Gain 1 Exposure time 50 ms and 200 ms; Prodan: UV light power 100% or 50% Gain 1 Exposure time 100 ms and 400 ms; BODIPY 500/510 and SeTau 488-NHS (50l-M): FITC light power 100% Gain 1 Exposure time 50 ms and 200 ms; DRAQ5: CY5 light power 100% Gain 1 Exposure time 200 ms and 400 ms; Cascade Blue and Pacific Blue-NHS Ester: UV light power 100% or 50% Gain 1 Exposure time 50 ms and 200 ms; Seta 375-NHS: UV light power 100% Gain 1 Exposure time 200 ms; Sulfo-Cy5.5 acid: CY5.5 light power 20% Gain 1 Exposure time 50 ms; Acridine Orange: CY5 light power 100% Gain 1 Exposure time 400 ms; Cytrak Orange: FITC light power 7% Gain 1 Exposure time 400 ms.

Tested conditions and results are presented in Table 6.

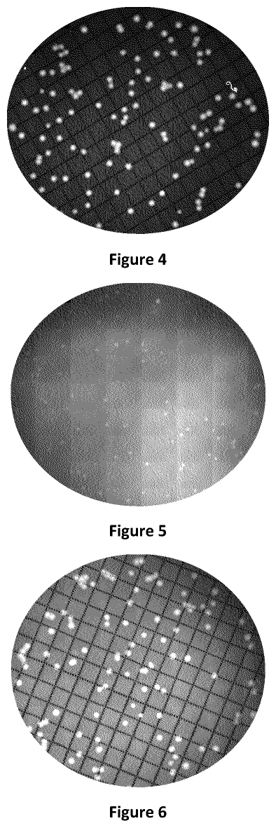

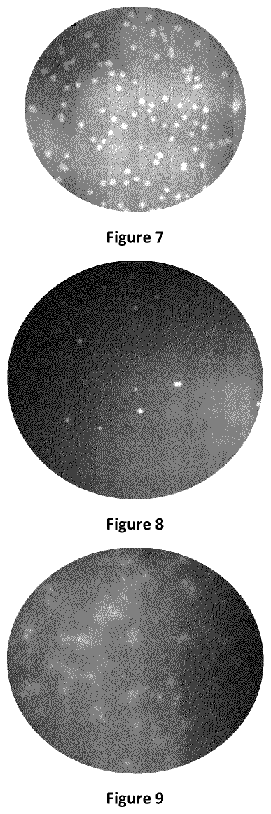

TABLE 6
Labelling and detection of aerobic mesophilic bacterial strains

| | | Bacterial | Labelling |
|---|---|---|---|
| Fluorescent dye | Membrane | strains | and detection |
| Acridine orange | MCE | E. coli | + |
| Cytrak Orange | MCE | E. coli | + |
| DRAQ5 | PVDF | E. faecalis | + |
| | | E. coli | + |
| | MCE | E. faecalis | + |
| | | E. coli | + |
| AF350-NHS Ester | PVDF | E. coli | + (FIG. 3) |
| | MCE | E. coli | + (FIG. 2) |
| Pacific Blue-NHS Ester | MCE | E. coli | + |
| Sulfo-Cy5.5 acid | MCE black | E. coli | + |
| Sulfo-Cy5-Acid | PVDF | E. coli | + (FIG. 7) |
| | | E. faecalis | + |
| | | P. aeruginosa | + |
| | | B. subtilis | + |
| | | S. aureus | + |
| | | M. extorquens | + |
| | MCE | E. coli | + (FIG. 6) |
| | | E. faecalis | + |
| BODIPY 500/510 | PVDF | E. coli | + |
| | MCE | E. coli | + |
| DAPI | PVDF | P. aeruginosa | + |
| | MCE | E. coli | + |
| | | P. aeruginosa | + |
| | | B. cepacia | + |
| | | S. aureus | + |
| | | M. extorquens | + |
| Prodan | PVDF | E. coli | + |
| | MCE | E. coli | + |
| AF405-DBCO | MCE | E. coli | + (FIG. 4) |
| Cascade Blue | MCE | E. coli | + |
| Seta 650-DBCO | PVDF | E. coli | + |
| Seta 375-NHS | MCE black | E. coli | + |
| SeTau 488-NHS | PVDF | E. coli | + |
| SeTau 647-Maleimide | PVDF | E. coli | + |
| Alexa Fluor 488-Acid | PVDF | E. coli | + |
| | | S. aureus | + |
| | MCE | E. coli | + |
| Alexa Fluor 488-DBCO | PVDF | E. coli | + |
| MB660R-Acid | PVDF | E. coli | + |
| | | S. aureus | + |
| | MCE | E. coli | + |
| MB660R-DBCO | PVDF | A. baumannii | + |
| | | B. cepacia | + |
| | | B. subtilis | + |
| | | C. sakazakii | + |
| | | E. coli | + (FIG. 5) |
| | | E. faecalis | + |
| | | K. pneumoniae | + |
| | | K. rhizophila | + |
| | | P. aeruginosa | + |
| | | S. aureus | + |
| | | S. sonnei | + |
| | | S. typhimurium | + |
| | | M. extorquens | + |
| | MCE | M. extorquens | + |
| ATTO647-Acid | PVDF | E. coli | + |
| | | S. aureus | + |

### Example 6: Detection of Yeasts and Molds

Strains of yeasts and molds used in this example are Aspergillus brasiliensis ATCC® 16404™, Penicillium variotii ATCC 1850Z Penicillium chrysogenum ATCC® 10106™, Candida albicans ATCC® 10231™, Geotrichum candidum, Saccharomyces cerevisiae ATCC® 9763™ and Zygosaccharomyces bailii DSM 70492.

200 μl of MB660R-DBCO (250 μM), Alexa Fluor 488-acid (50 μM), MB660R-acid (250 μM) or ATTO647-acid (5 μM), Seta 375-NHS (5 μM), 150 μL of Acridine Orange (40 μM), BODIPY 500/510 (10 μM on PVDF membrane and 150 μM on MCE membrane), or 250 μl of DAPI (50 μM) were deposited on YM agar plates (Glucose 10 g/L, Malt extract 3 g/L, Peptone 5 g/L, Yeast extract 3 g/L, Agar 15 g/L, pH value 6.2±0.2) or SDA agar plates (Pancreatic Digest of Casein 5 g/L, Peptic Digest of Animal Tissue 5 g/L, Dextrose 40 g/L, Agar 15 g/L, pH value 5.6±0.2) for Zygosaccharomyces bailii, Saccharomyces cerevisiae and Aspergillus brasiliensis and SDA agar plates for Candida albicans, Penicillium variotii and Penicillium chrysogenum. Each agar plate comprised one drop of a fluorescence dye. A filtration membrane was then deposited above the fluorescent dye drop on each agar plate. Plates were then incubated 39 h at 25° C.

150 μL of a fluorescent dye selected from the group consisting of AF350-NHS Ester (50 μM), Pacific Blue-NHS Ester (10 μM), Sulfo-Cy5-acid (150 μM), Prodan (15 μM on PVDF membrane and 50 μM on MCE membrane), AF405-DBCO (50 μM), Cascade Blue (1 μM), Seta 650-DBCO (100 μM), SeTau 488-NHS (50 μM), SeTau 647-Maleimide (100 μM) were deposited on SDA agar plates. Each agar plate comprised one drop of a fluorescence dye. A filtration membrane was then deposited above the fluorescent dye drop on each agar plate. Plates were then incubated 21 h at 35° C.

After incubation, microcolonies were detected using a microcolony semi-automated counter (MICA, DIAMIDEX, DAPI: Light power 12% Gain 1 Exposure time 200 ms; MB660R-DBCO, MB660R-acid, ATTO647-acid: CY5 Light power 100% Gain 1 Exposure time 200 and 500 ms; AF488-Acid: FITC Light power 100% Gain 1 Exposure time 200 and 400 ms; Sulfo-CY5-Acid, Seta-650 DBCO: CY5 Light power 100% Gain 1 Exposure time 50 ms and 400 ms; AF350-NHS Ester, AF405-DBCO: UV Light power: 50% or 20% Gain 1 Exposure time 100 ms and 400 ms; SeTau 488-NHS: FITC light power 100% Gain 1 Exposure time 50 ms and 200 ms; SeTau 647-Maleimide: CY5 Light power 100% Gain 1 Exposure time 50 ms and 200 ms; Prodan: UV Light power 100% or 50% Gain 1 Exposure time 100 ms and 400 ms; Cascade Blue and Pacific Blue-NHS Ester: UV light power 100% or 50% Gain 1 Exposure time 50 ms and 200 ms; Seta 375-NHS: UV light power 100% Gain 1 Exposure time 200 ms; Acridine Orange: CY5 light power 100% Gain 1 Exposure time 400 ms; BODIPY 500/510: FITC light power 100% Gain 1 Exposure time 50 ms and 200 ms).

Tested conditions and results are presented in Table 7.

TABLE 7
Labelling and detection of yeast and mold strains

| | | | Labelling and |
|---|---|---|---|
| Fluorescent dye | Membrane | Bacterial strains | detection |
| AF350-NHS ester | PVDF | C. albicans | + |
| | | A. brasiliensis | + |
| | MCE | C. albicans | + |
| | | A. brasiliensis | + |
| Pacific Blue-NHS | MCE | C. albicans | + |
| Ester | | A. brasiliensis | + |
| Sulfo-Cy5-Acid | PVDF | C. albicans | + |
| | | A. brasiliensis | + |
| | MCE | C. albicans | + |
| | | A. brasiliensis | + |
| DAPI | MCE | S. cerevisiae | + |
| | | Z. bailii | + |
| Prodan | PVDF | C. albicans | + |
| | MCE | C. albicans | + |
| | | A. brasiliensis | + |
| AF405-DBCO | PVDF | C. albicans | + |
| | MCE | C. albicans | + |
| | | A. brasiliensis | + |
| Cascade Blue | MCE | C. albicans | + |
| | | A. brasiliensis | + |
| Seta 650-DBCO | PVDF | C. albicans | + |
| | | A. brasiliensis | + |
| Seta 375-NHS | MCE black | C. albicans | + |
| SeTau 488-NHS | PVDF | C. albicans | + |
| | | A. brasiliensis | + |
| SeTau 647- | PVDF | C. albicans | + |
| Maleimide | | A. brasiliensis | + |
| AF488-Acid | PVDF | A. brasiliensis | + |
| | | S. cerevisiae | + |
| | | Z. bailii | + |
| | MCE | S. cerevisiae | + |
| | | Z. bailii | + |
| MB660R-Acid | PVDF | A. brasiliensis | + |
| | | S. cerevisiae | + |
| | | Z. bailii | + |
| | MCE | S. cerevisiae | + |
| | | Z. bailii | + |
| MB660R-DBCO | PVDF | C. albicans | + (FIG. 8) |
| | | S. cerevisiae | + |
| | | Z. bailii | + |
| | | A. brasiliensis | + (FIG. 9) |
| | | G. candidum | + |
| | | P. variotii | + |
| | | P. chrysogenum | + |
| ATTO647-Acid | PVDF | S. cerevisiae | + |
| Acridine orange | MCE | A. brasiliensis | + |
| BODIPY 500/510 | PVDF | C. albicans | + |
| | MCE | C. albicans | + |
| | | A. brasiliensis | + |

A mixed culture comprising C. albicans and A. brasiliensis was filtered on 0.45 μm PVDF or MCE membrane (47 mm). 150 μL of a fluorescent dye selected from the group consisting of AF350-NHS Ester (50 μM), Sulfo-Cy5-acid (150 μM), AF405-DBCO (50 μM), Seta 650-DBCO (100 μM) or SeTau 647-Maleimide (100 μM), were deposited on SDA agar plates. Each agar plate comprised one drop of a fluorescence dye. A filtration membrane was then deposited above the fluorescent dye drop on each agar plate. Plates were then incubated 21 h at 35° C. After incubation, microcolonies were detected using a microcolony semi-automated counter (MICA, DIAMIDEX, Sulfo-CY5-Acid, Seta-650 DBCO: CY5 Light power 100% Gain 1 Exposure time 50 ms and 400 ms; AF350-NHS Ester, AF405-DBCO: UV Light power: 50% or 20% Gain 1 Exposure time 100 ms and 400 ms; SeTau 647-Maleimide: CY5 Light power 100% Gain 1 Exposure time 50 ms and 200 ms).

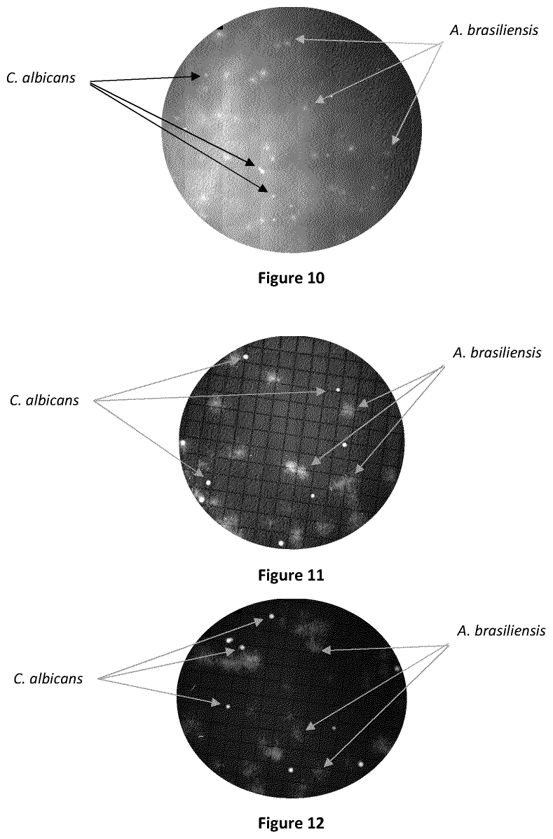

Results are presented in Table 8 below and FIGS. 10 to 13.

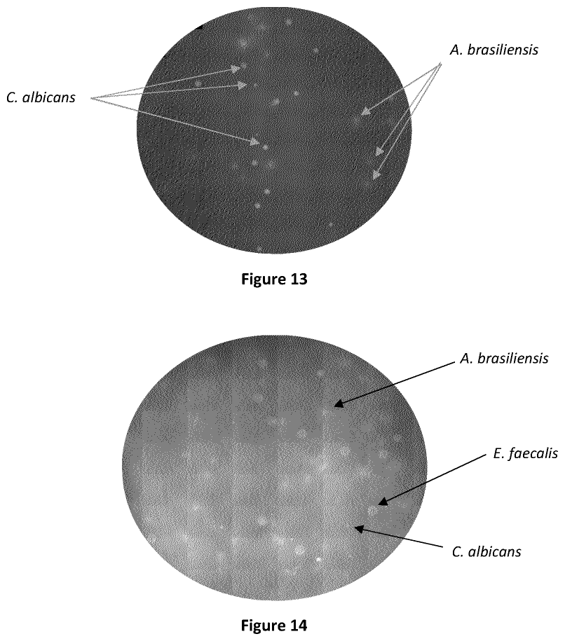

TABLE 8
Labelling and detection of a mix of C. albicans and A. brasiliensis

| | | Labelling and detection |
|---|---|---|
| Fluorescent dye | Membrane | of the two strains |
| AF350-NHS Ester | PVDF | + (FIG. 13) |
| | MCE | + (FIG. 12) |
| Sulfo-Cy5-Acid | PVDF | + (FIG. 10) |
| AF405-DBCO | MCE | + (FIG. 11) |
| Seta650-DBCO | PVDF | + |
| SeTau647-Maleimide | PVDF | + |

### Example 7: Detection of Gram Negative Bacteria in UTI Diagnosis

Gram negative bacteria used in this example is Escherichia coli ATCC® 8739™.

200 μL of a mix of MB660R-DBCO (100 μM) and Sulfo-Cy5-Acid (100 μM) were deposited on MacConkey agar plates (peptone 20 g/L, lactose 10 g/L, Bile salts 1.5 g/L, crystal violet 0.001 g/L, neutral red 0.05 g/L, sodium chloride 5 g/L, Agar 15 g/L, pH value 7.1+/−0.2). Each agar plate comprised one drop of the mix of dyes.

A filtration membrane was then deposited above the fluorescent dye drop on each agar plate. Plates were then incubated 8 h at 35° C.

After incubation, microcolonies were detected using a microcolony semi-automated counter (MICA, DIAMIDEX, Light power 100%, Gain 1, Exposure time 200 ms).

E. coli colonies were labelled and detected in each condition.

### Example 8: Total Count of Microorganisms (Bacteria, Yeasts and Molds)

Strains used in this example are Cutibacterium acnes ATCC® 6919™, Methylobacterium extorquens ATCC® 43645™, Enterococcus faecalis ATCC® 29212™, Aspergillus brasiliensis ATCC® 16404™ Candida albicans ATCC®10231™ and Weissella confusa ATCC® 10881™.

A mixed culture comprising Enterococcus faecalis, Aspergillus brasiliensis and Candida albicans was filtered on 0.45 μm PVDF membrane (47 mm). For the mixture, 150 μL of a mix of MB660R-DBCO (100 μM) and Sulfo-Cy5-Acid (100 μM) were deposited on TSA or SDA agar plates. Each agar plate comprised one drop of the mix of dyes. A filtration membrane was then deposited above the fluorescent dye drop on each agar plate. Plates were then incubated 24 h at 35° C. for TSA plates and at 25° C. for SDA plates. After incubation, microcolonies were detected using a microcolony semi-automated counter (MICA, DIAMIDEX, Cy5 Light power 100%, Gain 1, Exposure time 50 and 200 ms).

A mixed culture comprising Aspergillus brasiliensis and Candida albicans and a culture of Weissella confusa were filtered on 0.45 μm PVDF membrane (47 mm). For these cultures, 150 μL of a mix of MB660R-DBCO (100 μM) and Sulfo-Cy5-Acid (100 μM) were deposited on OSA plates (casein peptone 10 g/L, dipotassium hydrogen phosphate 3 g/L, D(+)-glucose 4 g/L, orange extract 5 g/L, yeast extract 3 g/L, agar 17 g/L, pH value 5.5±0.2). Each agar plate comprised one drop of the mix of dyes. A filtration membrane was then deposited above the fluorescent dye drop on each agar plate. Plates were then incubated 24 h at 30° C. After incubation, microcolonies were detected using a microcolony semi-automated counter (MICA, DIAMIDEX, Cy5 Light power 100%, Gain 1, Exposure time 200 ms).

A culture of Methylobacterium extorquens was filtered on 0.45 μm PVDF membrane (47 mm). For this culture, 150 μL of a mix of MB660R-DBCO (100 μM) and Sulfo-Cy5-Acid (100 μM) were deposited on TSA or R2A agar plates (casein acid hydrolysate 0.5 g/L, dextrose 0.5 g/L, dipotassium phosphate 0.3 g/L, magnesium sulfate 0.024 g/L, proteose peptone 0.5 g/L, sodium pyruvate 0.3 g/L, starch, soluble 0.5 g/L, yeast extract 0.5 g/L, agar 15 g/L, pH value 7.2±0.2). Each agar plate comprised one drop of the mix of dyes. A filtration membrane was then deposited above the fluorescent dye drop on each agar plate. Plates were then incubated 96 h at 25° C.

A culture of Cutibacterium acnes was filtered on 0.45 μm PVDF membrane (47 mm). For this culture, 150 μL of a mix of MB660R-DBCO (100 μM) and Sulfo-Cy5-Acid (100 μM) were deposited on TSA. Each agar plate comprised one drop of the mix of dyes. A filtration membrane was then deposited above the fluorescent dye drop on each agar plate. Plates were then incubated 96 h at 35° C., 5% CO₂.

After incubation, microcolonies were detected using a microcolony semi-automated counter (MICA, DIAMIDEX, CY5 Light power 100%, Gain 1 Exposure time 50 ms and 200 ms).

Results are presented in Table 9.

TABLE 9
Labelling and detection of multiple targeted strains
using a mix of MB660R-DBCO and Sulfo-Cy5-Acid

| | Labelling and detection |
|---|---|
| Strains | of the strains |
| Methylobacterium extorquens | + |
| (on TSA or R2A agar plates) | |
| Cutibacterium acnes | + |
| Enterococcus faecalis + Candida albicans + | + (FIG. 14) |
| Aspergillus brasiliensis | |
| (on TSA or SDA agar plates) | |
| Candida albicans + Aspergillus brasiliensis | + |
| Weissella confusa | + |

### Example 9: Comparison of the Method of the Invention and a Method without Fluorescent Dye

Strains used in this example are Escherichia coli ATCC® 8739™, Aspergillus brasiliensis ATCC® 16404™ and Candida albicans ATCC® 10231™.

150 μL of a fluorescent dye selected from the group consisting of DRAQ5 (125 μM), AF350-NHS Ester (50 μM), Sulfo-Cy5-acid (150 μM), BODIPY 500/510 (10 μM), Prodan (151 μM on PVDF membrane and 50 μM on MCE membrane), AF405-DBCO (50 μM), Seta 650-DBCO (100 μM) or SeTau 647-Maleimide (100 μM) were deposited on TSA agar plates for Escherichia coli and on SDA agar plates for Aspergillus brasiliensis and Candida albicans. A filtration membrane was then deposited above the fluorescent dye drop on each agar plate. Plates were then incubated 18 h at 35° C. for Escherichia coli and 21 h at 35° C. for Aspergillus brasiliensis and Candida albicans. After incubation, microcolonies were detected using a microcolony semi-automated counter (MICA, DIAMIDEX, Sulfo-CY5-Acid, Seta-650 DBCO: CY5 Light power 100% Gain 1 Exposure time 50 ms and 400 ms; AF350-NHS Ester, AF405-DBCO: UV Light power: 50% or 20% Gain 1 Exposure time 100 ms and 400 ms; SeTau 647-Maleimide: CY5 Light power 100% Gain 1 Exposure time 50 ms and 200 ms; Prodan: UV Light power 100% or 50% Gain 1 Exposure time 100 ms and 400 ms; BODIPY 500/510: FITC Light power 100% Gain 1 Exposure time 50 ms and 200 ms; DRAQ5: CY5 Light power 100% Gain 1 Exposure time 200 ms and 400 ms).

To compare results obtained with the method of the invention and results obtained with standard method, strains used in this example (Escherichia coli ATCC®8739™, Aspergillus brasiliensis ATCC®16404™ and Candida albicans ATCC®10231™) were filtered on 0.451 μm MCE membrane (47 mm). Filtration membranes were then deposited on TSA for Escherichia coli or SDA agar plate for Aspergillus brasiliensis and Candida albicans. Plates were then incubated 3 to 4 days at 35° C. After incubation, colonies were counted on eye.

Results are presented in Table 10.

TABLE 10
Enumeration of colonies using the method of the invention and a standard method

| | | Counting Scan | Counting Scan | Counting Picture |
|---|---|---|---|---|
| | | at 17 h on MCE | at 17 h on PVDF | at Day 3 on MCE |
| | | membrane with | membrane with | membrane without |
| | | fluorochrome | fluorochrome | fluorochrome |
| E. coli | Sulfo-CY5-Acid | 114 | 120 | 103 |
| | Prodan | 118 | 126 | 91 |
| | BODIPY | / | 112 | 92 |
| | AF350-NHS Ester | 109 | 107 | 98 |
| | AF405-DBCO | 112 | / | 98 |
| | SeTau 647-Maleimide | 116 | 189 | 157 |
| | DRAQ5 | 185 | 162 | 157 |
| | Seta 650-DBCO | / | 170 | 157 |
| C. albicans | Sulfo-CY5-Acid | 13 | 13 | 14 |
| | Prodan | 11 | 17 | 19 |
| | AF350-NH5 Ester | 9 | 20 | 7 |
| | AF405-DBCO | 8 | 13 | 7 |
| | SeTau 647-Maleimide | / | 10 | 6 |
| | Seta 650-DBCO | / | 6 | 6 |
| A. brasiliensis | Sulfo-CY5-Acid | 25 | 34 | 18 |
| | Prodan | / | 6 | 18 |
| | AF350-NH5 Ester | 25 | 19 | 18 |
| | AF405-DBCO | 21 | 11 | 18 |
| | SeTau 647-Maleimide | / | 36 | 15 |
| | Seta 650-DBCO | / | 36 | 15 |

(/): indicates that the condition has not been tested

As shown, in this table, the method of the invention allows earlier detection of microorganisms as well as more accurate enumeration than the standard method.

### Example 10: Enumeration of Microorganisms Implicated in Purified Water and Pharmaceutical Water System

Water is a key part of the pharmaceutical industry. Water is used for cleaning; as an ingredient for aqueous sterile and non-sterile products; for hand washing; and as the steam supply to autoclaves, among other uses. Due to its criticality in pharmaceutical production, microbiological control of water is of great importance. Because water is ever present, each grade of pharmaceutical water is a potential source of microbiological contamination, especially when not properly controlled.

In pharmaceutical 3 types of water are present:
a) Mains (or potable) Water;
b) Purified Water;
c) WFI (Water-for-Injection).

Each of these is of a different grade and the list descends with an increased expectation of microbial control (that is tighter limits apply to Water-for-Injection that for mains water). Mains water is supplied by a utility company and is of “drinking water” (potable water) quality. The results from the monitoring of water systems are assessed from heterotrophic microbial counts against pre-defined alert and action levels.

For the microbiological examination of the water, the method of testing was membrane filtration through the use of 0.45 μm filter. The filters were placed onto R2A agar and subjected to a temperature regime of 20-25° C. for fourteen days for mains water and 30-35° C. for five days for the purified water and WFI. The reason for these different regimes is that the European Pharmacopeia, for GMP facilities, requires the use of the stated temperature and time. For mains water, a pharmaceutical facility can select the cultural conditions. Selecting optimal cultural conditions relates to a classic dilemma in microbiology of concerning the most appropriate temperature, time and culture media to use. R2A was formulated, with a low level nutrient, so that it would detect a higher proportion of heterotrophic bacteria. The theory was that as bacteria in water are subject to nutritive depleted conditions, then they would be more likely to grow on growth media prepared to more closely match those prevalent conditions.

A metadata study covering a fifteen year review period (2000 to 2014) with 54140 samples collected and tested was performed and presented a review of the typical culturable microorganisms recoverable from pharmaceutical water systems (Tim Sandle, September 2015. SOJ Microbiology & Infectious Diseases 3(2):1-8).

According to this metadata analysis and pharmacopeias recommendations, a list of microorganisms was tested. Gram negative bacteria used in this example were Acinetobacter baumanii ATCC 19606, Aeromonas hydrophila ATCC 35654, Brevundimonas diminuta ATCC 19146, Burkholderia cepacia ATCC 25608, Citrobacter freundii ATCC 8090, Edwardsiella tarda ATCC 15947, Escherichia coli ATCC 8739, Enterobacter aerogenes ATCC 35028, Ochrobactrum anthropi CIP 82.115, Klebsiella pneumoniae ATCC 13883, Methylobacterium extorquens CIP 106787, Moraxella osloensis ATCC 19976, Pantoea agglomerans ATCC 27155, Proteus mirabilis ATCC 29906, Pseudomonas aeruginosa ATCC 10145, Pseudomonas fluorescens ATCC 13525, Ralstonia pickettii ATCC 27511, Salmonella typhimurium ATCC 13311, Serratia marcescens ATCC 13880, Shigella sonnei ATCC 25931, Sphingomonas paucimobilis ATCC 29837, Stenotrophomonas maltophilia ATCC 13637, Yersinia enterocolitica ATCC 9610. Gram positive bacteria are Bacillus subtilis ATCC 6633, Enterococcus faecalis ATCC 19433, Staphylococcus aureus ATCC 6538. Yeasts and molds tested were Aspergillus brasiliensis ATCC® 16404™ and Candida albicans ATCC® 10231™. This selection covers at least 84% of mains water systems microbial contaminants, 77% of purified water systems contaminants and the most prevalent contaminants of pharmaceutical WFI systems.

150 μL of a mix of MB660R-DBCO (100 μM) and Sulfo-Cy5-Acid (100 μM) were deposited on Reasoner's 2A Agar (R2A) plates (Yeast Extract 0.5 g/L, Proteose Peptone 0.5 g/L, Casein Hydrolysate 0.5 g/L, Glucose 0.5 g/L, Starch 0.5 g/L, Dipotassium Hydrogen Phosphate 0.3 g/L, Magnesium Sulphate, Anhydrous 0.024 g/L, Sodium Pyruvate 0.3 g/L, Agar 15.0 g/L, final pH 7.2±0.2), on TSA agar plates (Tryptone 15 g/L, Soja papainic peptone 5 g/L, Sodium chloride 5 g/L, Agar 15 g/L, pH value 7.3±0.2) or SDA agar plates (Pancreatic Digest of Casein 5 g/L, Peptic Digest of Animal Tissue 5 g/L, Dextrose 40 g/L, Agar 15 g/L, pH value 5.6±0.2). Alternatively, 150 μl of a mix of AF350-NHS Ester (150 μM) and AF405-DBCO (150 μM) were deposited in same conditions on same set of microorganisms. Each agar plate comprised one drop of the mix of dyes.

Each microbial culture was filtered on 0.45 μm PVDF or MCE membrane (47 mm). Each membrane was then deposited above the fluorescent dye drop on each agar plate. Plates were then incubated at 32.5° C. for the minimal timing necessary to enumerate all microcolonies spiked on membranes with a microcolony semi-automated counter (MICA, DIAMIDEX, when MB660R-DBCO and Sulfo-Cy5-Acid were used: Cy5 Light power 100%, Gain 1, Exposure time 100 and 200 ms; when AF350-NHS Ester and AF405-DBCO were used: UV Light power 100%, Gain 1, Exposure time 50 and 100 ms). The results are presented on Tables 11 and 12 below.

TABLE 11
Labelling with a mix of AF350-NHS Ester and AF405-DBCO
dyes and detection of microorganisms implicated in
purified water and Pharmaceutical Water System

| | Culture | | Labelling |
|---|---|---|---|
| Membrane | conditions | Microorganism | and detection |
| PVDF | R2A 32.5° C. | B. subtilis | X |
| | | M. extorquens | X |
| | | S. aureus | X |
| | | P. aeruginosa | X |
| | | C. albicans | X |
| | | R. picketii | X |
| | | B. diminuta | X |
| | | A. baumanii | X |
| | | A. hydrophila | X |
| | TSA 32.5° C. | B. cepacia | X |
| | | B. subtilis | X |
| | | A. brasiliensis | X |
| | | M. extorquens | X |
| | | S. aureus | X |
| | | P. aeruginosa | X |
| | | C.albicans | X |
| | | R. picketii | X |
| | | A. hydrophila | X |
| | | B. diminuta | X |
| | SDA 32.5° C. | C. albicans | X |
| black MCE | R2A 32.5° C. | A. brasiliensis | X |
| | | B. cepacia | X |
| white MCE | | A. brasiliensis | X |
| | | B. cepacia | X |

TABLE 12
Labelling with a mix of MB660R-DBCO and Sulfo-Cy5-Acid
dyes and detection of microorganisms implicated in
purified water and Pharmaceutical Water System

| | Culture | | Labelling |
|---|---|---|---|
| Membrane | conditions | Microorganism | and detection |
| PVDF | TSA 32.5° C. | B.cepacia | X |
| | | B.subtilis | X |
| | | A.brasiliensis | X |
| | | M.extorquens | X |
| | | S.aureus | × |
| | | P.aeruginosa | X |
| | | C.albicans | X |
| | | B.diminuta | X |
| | | R. picketii | X |
| | SDA 32.5° C. | A. brasiliensis | X |
| | | C. albicans | X |
| | R2A 32.5° C. | B. subtilis | X |
| | | S. aureus | X |
| | | E. coli | X |
| | | M. extorquens | X |
| | | B. cepacia | × |
| | | P aeruginosa | X |
| | | P. fluorescens | × |
| | | A. brasiliensis | × |
| | | C. albicans | × |
| | | M. extorquens | X |
| | | B.diminuta | X |
| | | A.baumanni | X |
| | | A.hydrophila | X |
| | | C. freundii | X |
| | | E. aerogenes | X |
| | | P. agglomerans | X |
| | | S. maltophilia | X |
| | | S. marcescens | X |
| | | S. sonnei | X |
| | | Y. enterocolitica | X |
| | | A. faecalis | X |
| | | E. tarda | X |
| | | K. pneumoniae | X |
| | | M. osloensis | X |
| | | O. anthropi | X |
| | | P. mirabilis | X |
| | | R. picketii | X |
| | | S. paucimobilis | X |
| | | S. typhimurium | X |

### Example 11: Methods of Staining with a Mix of Fluorochromes

Addition of the fluorescent dyes to the bacterial culture before filtration Bacillus subtilis ATCC 6633 was diluted in NaCl 0.9% from cryopreserved stocks previously quantified to obtain a defined number of microorganisms per experimental condition. A mix of MB660R-DBCO (50 μM) and Sulfo-Cy5-acid (50 μM) was added to the suspensions in a final volume of 10 ml of NaCl 0.9%. The resulting suspensions were filtered on white or black MCE membranes and micro-organisms were allowed to grow 16 h at 32.5° C. on TSA plates (Tryptone 15 g/L, Soja papainic peptone 5 g/L, Sodium chloride 5 g/L, Agar 15 g/L, pH value 7.3±0.2).

### Incorporation of the Fluorescent Dyes in the Agar Medium

150 μl of a mix constituted by MB660R-DBCO (50 μM) and Sulfo-Cy5-acid (50 μM) was streaked on the surface of TSA plates (Tryptone 15 g/L, Soja papainic peptone 5 g/L, Sodium chloride 5 g/L, Agar 15 g/L, pH value 7.3±0.2) or was added and mixed to melt TSA agar medium before pouring said medium on plates. Agar plates were kept at 4° C. for 12 days. The day of experiment, Bacillus subtilis ATCC 6633 was diluted in NaCl 0.9% from cryopreserved stocks previously quantified to obtain a defined number of microorganisms per experimental condition. The resulting suspensions were filtered on white MCE membranes and micro-organisms were allowed to grow 16 h à 32.5° C. on TSA plates with incorporated mix of fluorochromes.

### Fluorescent Dyes Deposited Between the Membrane and the Agar Medium

Bacillus subtilis ATCC 6633 was diluted in NaCl 0.9% from cryopreserved STOCKS previously quantified to obtain a defined number of microorganisms per experimental condition. The resulting suspensions were filtered on white or black MCE membranes. 150 μl of a mix constituted by MB660R-DBCO (50 μM) and Sulfo-Cy5-acid (50 μM) was deposited on TSA agar plates. A filtration membrane was then deposited above the fluorescent dye drop on each agar plate and micro-organisms were allowed to grow 16 h at 32.5° C. on TSA plates.

For each protocol, after incubation, microcolonies were detected using a microcolony semi-automated counter (MICA, DIAMIDEX, Sulfo-CY5-Acid and MB660R-DBCO: CY5 Light power 10%-20% Gain 1 Exposure time 50 ms and 100 ms).

Each of these methods allows an efficient staining and detection of the microorganisms. The results of these experiments are illustrated in FIG. 16.

### Example 12: Methods of Detection Avoiding Disconnection from Agar Media

Several strains of Alicyclobacillus bacteria including Alicyclobacillus acidoterrestris ATCC® 49025™, Alicyclobacillus cycloheptanicus ATCC® 49028™ and Alicyclobacillus herbarius DSM 13609 were used in this example. Strains were diluted in NaCl 0.9% from cryopreserved stocks previously quantified to obtain a defined number of microorganisms per experimental condition.

### Protocol 1: The Detection was Done on Membranes Placed on the Agar Medium.

150 μL of a fluorescent dye MB660R-DBCO (100 μM) was deposited on BAT agar plates (Yeast extract 2 g/l, D(+) glucose 5 g/l, Calcium chloride 0.25066 g/l, Magnesium sulfate 0.5 g/l, Ammonium sulfate 0.2 g/l, Potassium dihydrogen phosphate 3 g/l, Zinc sulfate 0.00018 g/l, Copper sulfate 0.00016 g/l, Manganese sulfate 0.00015 g/l, Sodium molybdate dihydrate 0,00030 g/l, Agar-Agar 18 g/l, pH value 3.8-4.2). Each agar plate comprised one drop of a fluorescence dye. Alicyclobacillus cycloheptanicus and Alicyclobacillus herbarius were filtrated on PVDF filtration membranes. Membranes were then deposited above the fluorescent dye drop on each agar plate.

### Protocol 2: The Detection was Done Directly on the Agar Medium.

A 250 μl drop containing A. acidoterrestris diluted in purified water and 12.5 μL of a fluorescent dye MB660R-DBCO (250 μM) was streaked on the surface of the agar media. Plates were then incubated 24 h at 45° C.

For each protocol, after incubation, microcolonies were detected using a microcolony semi-automated counter (MICA, DIAMIDEX, Light power 100%, Gain 1, Exposure time 100 for detection done on membrane on agar media; Light power 100%, Gain 1, Exposure time 50 for detection directly on agar media).

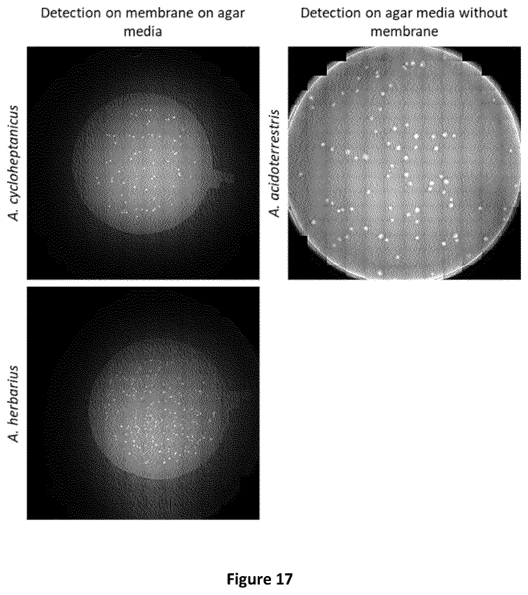

These two protocols allow an efficient staining and detection of the microorganisms. The results of these experiments are illustrated in FIG. 17.

### Example 13: Enumeration of Bacteria Implicated in Uncomplicated and Complicated UTI

The most common causative agent for both uncomplicated and complicated UTIs is Escherichia coli. For the agents involved in uncomplicated UTIs, Escherichia coli is followed in prevalence by Klebsiella pneumoniae, Staphylococcus saprophyticus, Enterococcus faecalis, group B Streptococcus (GBS), Proteus mirabilis, Pseudomonas aeruginosa, Staphylococcus aureus and Candida spp. For complicated UTIs, the order of prevalence for causative agents, following Escherichia coli as most common, is Enterococcus spp., K. pneumoniae, Candida spp., S. aureus, P. mirabilis, P. aeruginosa and GBS.

Gram negative bacteria used in this example were E. coli ATCC®8739™, K. pneumoniae ATCC® 13883™ and P. mirabilis ATCC® 29906™. Gram positive bacteria were E. faecalis ATCC® 19433™ and S. aureus ATCC® 6538™. This selection covers 89% of uncomplicated and complicated UTI.

150 μL of a mix of MB660R-DBCO (100 μM) and Sulfo-Cy5-Acid (100 μM) were deposited on TSA agar plates (Tryptone 15 g/L, Soja papainic peptone 5 g/L, Sodium chloride 5 g/L, Agar 15 g/L, pH value 7.3±0.2) or Columbia blood agar plates (Peptone 23 g/l, Starch 1 g/l, Sodium chloride 5 g/l, Sheep Blood 50 ml/l, Agar 14 g/l, pH value 7.3±0.2) for Gram positive bacteria and P. mirabilis and on TSA agar plates, Columbia blood agar plates or MacConkey agar plates (peptone 20 g/L, lactose 10 g/L, Bile salts 1.5 g/L, crystal violet 0.001 g/L, neutral red 0.05 g/L, sodium chloride 5.0 g/L, Agar 15.0 g/L, pH value 7.1+/−0.2) for coliforms (E. coli and K. pneumoniae). Alternatively, 150 μl of DAPI (50 μM) was deposited in same conditions on same set of microorganisms. Each agar plate comprised one drop of the mix of dyes.

Each bacterium was diluted from cryopreserved stocks previously quantified to obtain a defined number of microorganisms per experimental condition. The resulting suspensions were filtered on PVDF or MCE membranes.

A filtration membrane was then deposited above the fluorescent dye drop on each agar plate. Plates were then incubated at 37° C. for the minimal timing necessary to enumerate all microcolonies spiked on membranes with a microcolony semi-automated counter (MICA, DIAMIDEX, MB660R-DBCO and Sulfo-Cy-Acid Cy5 Light power 100%, Gain 1, Exposure time 100 and 200 ms; DAPI: UV Light power 100%, Gain 1, Exposure time 50 and 0.100%)

TABLE 13
Minimal timing necessary to enumerate microcolonies
and percentage of detected objects compared to
standard procedure timing (24 h at 37° C.)

| | | Time detection, membrane | | | |
|---|---|---|---|---|---|
| | | and fluorescent dye(s) | | | |
| | | MB660R-DBCO and | | DAPI/ | |
| | Culture | Sulfo-Cy5-Acid/ | | black MCE | |
| | medium | PVDF membrane | | membrane | |
| K. pneumonia | TSA | 10 h | (100%) | 10 h | (100%) |
| E. coli | | 7 h | (89%) | 6 h | (100%) |
| P. mirabilis | | 10 h | (89%) | 10 h | (98%) |
| S. aureus | | 10.5 h | (100%) | 8 h | (100%) |
| E. faecalis | | 9 h | (100%) | 7 h | (100%) |
| K. pneumonia | Columbia | 10 h | (100%) | 10 h | (100%) |
| E. coli | Blood | 7 h 30 | (99%) | 7 h 30 | (100%) |
| P. mirabilis | | 10 h 30 | (100%) | 10 h 30 | (94%) |
| S. aureus | | 10 h 45 | (100%) | 10 h 45 | (100%) |
| E. faecalis | | 10 h 30 | (100%) | 10 h 30 | (100%) |
| K. pneumoniae | MacConkey | 10 h | (96%) | 11 h | (100%) |
| E. coli | | 10.5 h | (94%) | 10 h | (98%) |

As shown in Table 13, E. coli responsible of 75% of uncomplicated UTI and 65% of complicated UTI can be enumerated with the method of the invention in less than 6 h on TSA agar plates, less than 7 h 30 on Columbia agar plates and 10 h on MacConkey agar plates. Furthermore, the most prevalent microorganisms implicated in uncomplicated and complicated UTI can be enumerated in less than 11 h on TSA agar plates Columbia blood agar plates and MacConkey agar plates.

1-55. (canceled)

56. A method for detecting or enumerating a microorganism or a group of microorganisms of interest comprising
a) contacting, in a container, a sample suspected of containing said microorganism or group of microorganisms with a solid growth medium containing nutrients to support growth of said microorganism or group of microorganisms and with at least one fluorescent dye;
b) incubating said container under conditions and for a time sufficient to form microcolonies of said microorganism or group of microorganisms; and
c) detecting or enumerating microcolonies emitting a fluorescent signal of said at least one fluorescent dye,
thereby detecting or enumerating said microorganism or group of microorganisms contained in the sample.

57. The method according to claim 56, wherein, before step a), the microorganisms of the sample are concentrated on a membrane filter and wherein in step a) said membrane and microorganisms are contacted with the solid growth medium and said at least one fluorescent dye.

58. The method according to claim 56, wherein step a) may comprise
a1) contacting a sample suspected of containing said microorganism or group of microorganisms with at least one fluorescent dye, and
a2) contacting, in a container, said sample with a solid growth medium containing nutrients to support growth of said microorganism or group of microorganisms,
steps a1) and a2) being carried out simultaneously or sequentially, in any order.

59. The method according to claim 58, wherein step a1) is carried out before step a2).

60. The method according to claim 59, wherein, before step a2), the microorganisms of the sample are concentrated on a membrane filter.

61. The method according to claim 57, wherein the membrane filter is made of mixed cellulose esters (MCE), polyvinylidene fluoride (PVDF), nitrocellulose, polytetrafluoroethylene, polycarbonate or nylon.

62. The method according to claim 61, wherein the membrane filter is made of MCE or PVDF.

63. The method according to claim 62, wherein the membrane filter is made of MCE.

64. The method according to claim 62, wherein the membrane filter is made of PVDF.

65. The method according to claim 57, wherein after step b) and before in step c), the membrane filter is removed from the solid growth medium and microcolonies thereon are detected and/or enumerated in step c).

66. The method according to claim 57, wherein in step c) microcolonies emitting a fluorescent signal are detected and/or enumerated using a fluorescence microscope or a solid-phase cytometer.

67. The method according to claim 56, wherein the solid growth medium is selective for said microorganism or group of microorganisms.

68. The method according to claim 56, wherein said at least one fluorescent dye is selected from the group consisting of xanthene-based dyes, acridine dyes, anthracene-based dyes, coumarin-based dyes, cyanine-based dyes, dipyrromethene-based dyes, naphthalene-based dyes, pyrene-based dyes, squaraine-based dyes, squaraine rotaxane-based dyes and 4′,6-diamidino-2-phenylindole (DAPI), and combinations thereof.

69. The method according to claim 68, wherein the microorganism or group of microorganisms of interest
belongs to thermophilic acidophilic bacteria, and is selected from the group consisting of Alicyclobacillus acidoterrestris, Alicyclobacillus acidocaldarius, Alicyclobacillus acidiphilus, Alicyclobacillus cycloheptanicus, Alicyclobacillus hesperidum, Alicyclobacillus herbarius and Alicyclobacillus contaminans, and combinations thereof; and/or
belongs to acetic acid bacteria, and is selected from bacteria belonging to the genera Acetobacter, Gluconobacter, Gluconacetobacter and Asaia, and combinations thereof; and/or
belongs to lactic acid bacteria, and is selected from bacteria belonging to the genera Lactobacillus and Weissella, and combinations thereof; and/or
belongs to of anaerobic bacteria, and is selected from Clostridium and Cutibacterium bacteria, and combinations thereof, and/or
belongs to aerobic mesophilic bacteria, and is selected from the genera Methylobacterium, Pseudomonas, Bacillus, Escherichia, Staphylococcus, Acinetobacter, Cronobacter, Klebsiella, Salmonella, Enterococcus, Shigella, Kocuria and Burkholderia, and combinations thereof, and/or
belongs to Gram negative bacteria, and is selected from the genera Escherichia and Pseudomonas, and combinations thereof, and/or
belongs to the group of heterotrophic bacteria, is selected from bacteria belonging to the genera Acinetobacter, Aeromonas, Brevundimonas, Burkholderia, Citrobacter, Edwardsiella, Enterobacter, Escherichia, Ochrobactrum, Klebsiella, Methylobacterium, Moraxella, Pantoea, Proteus, Pseudomonas, Ralstonia, Salmonella, Serratia, Shigella, Sphingomonas, Stenotrophomonas, Yersinia, Bacillus, Enterococcus, Micrococcus, Staphylococcus, and combinations thereof, and/or
belongs to the group of yeasts and molds, and is selected from the genera Candida, Zygosaccharomyces, Aspergillus, Saccharomyces, Geotrichum and Penicillium, and combinations thereof.

70. The method according to claim 56, wherein said at least one fluorescent dye is selected from the group consisting of MB660R, Acridine orange, DRAQ5, Cytrak Orange, Alexa Fluor 350, Pacific Blue, Cy5, BODIPY 500/510, prodan, Alexa Fluor 405, Cascade Blue, Seta 650, Seta 375, Setau 647, SeTau 488, Alexa Fluor 488, ATTO 647 and DAPI, derivatives thereof, and combinations thereof.
